(19) United States
(12) Patent Application Publication
(10) Pub. No.: US 2010/0199386 A1
(43) Pub. Date: Aug. 5, 2010

(54) CONTROL OF COLD-INDUCED SWEETENING AND REDUCTION OF ACRYLAMIDE LEVELS IN POTATO OR SWEET POTATO

(75) Inventors: Pudota Bala Bhaskar, Madison, WI (US); Jiming Jiang, Fitchburg, WI (US)

Correspondence Address: Lisa Mueller;c/o Polsinelli Shughart PC, 161 N. Clark Street, Suite 4200, Chicago, IL, 60601, US

(73) Assignee: WISCONSIN ALUMNI RESEARCH FOUNDATION, Madison, WI (US)

(21) Appl. No.: 12/698,275

(22) Filed: Feb. 2, 2010

Related U.S. Application Data
(60) Provisional application No. 61/149,397, filed on Feb. 3, 2009.
(60) Provisional application No. 61/241,876, filed on Sep. 12, 2009.

(51) Int. Cl. A01H 1/00; C07H 21/04; C12N 15/82; A01H 5/00; A23L 1/216

(52) U.S. Cl. 800/284; 536/23.1; 435/320.1; 800/298; 800/278; 426/637; 800/317.2

(57) ABSTRACT

The present invention is directed to methods and compositions to eliminate cold storage-induced sweetening of potato or sweet potato. The invention is accomplished in part by silencing the vacuolar acid invertase gene using RNAi technology. The resulting potatoes withstand cold storage without significant hexogenesis, producing potatoes or sweet potatoes that have reduced Maillard reactions when fried in hot oil. The fried products accumulate significantly lower levels of acrylamide compared to controls.

### RELATED APPLICATION INFORMATION

This application claims the benefit of U.S. Ser. No. 61/149,397 filed on Feb. 3, 2009 and to U.S. Ser. No. 61/241,876 filed on Sep. 12, 2009, the contents of each of which are herein incorporated by reference.

### GOVERNMENT SUPPORT

This invention was made with support from the National Science Foundation, Grant No. DBI-0218166 and USDA/CSREES 08-CRHF-0-6055. The US government may have certain rights in this invention.

### FIELD OF THE INVENTION

The present invention is generally directed to the inhibition of sugar conversion in potato or sweet potato during cold storage (2-12° C., especially 2-4° C.). Specifically, the invention is directed to silencing the vacuolar acid invertase gene using RNAi to inhibit the conversion of sucrose to fructose and glucose in potato tubers and to reduce the acrylamide levels in fried edible potato products or sweet potato products.

### SEQUENCE LISTING

The instant application contains a Sequence Listing which has been submitted via EFS-Web and is hereby incorporated by reference in its entirety. Said ASCII copy, created on Feb. 1, 2010, is named WARFPOTA.txt, and is 20,397 bytes in size.

### COMPACT DISC FOR SEQUENCE LISTINGS AND TABLES

Not applicable.

### BACKGROUND OF THE INVENTION

### Cold-Storage Induced Sweetening

Potato tubers (Solanum tuberosum) are stored at low temperatures ((47-50° F. (8-10° C.)) to prevent sprouting, reduce respiration and minimize disease losses (Rausch and Greiner, 2004). However, these temperatures are not ideal; colder storage temperatures are more preferable (2-4° C.) because colder temperatures would reduce (1) the need to use fungicides and bactericides in storage; (2) the loss of solids through respiration; (3) the need for chemical sprout suppressants; and (4) would help to increase the marketing window (Sowokinos, 2007). In the cold, however, starch, a polysaccharide, is converted into the simple reducing sugars glucose and fructose; a phenomenon recognized as cold-induced sweetening (CIS) (Dale and Bradshaw, 2003; Keijbets, 2008; Rausch and Greiner, 2004; Sowokinos, 2001; Sowokinos, 2007). The carbonyl groups of these sugars react with the amino group of free amino acids (a Maillard type reaction) as raw potatoes are fried in oil at high temperature, resulting in unacceptable dark and bitter-tasting chips and fries (Sowokinos, 2007).

In addition, this reaction also produces acrylamide, a toxin and potential carcinogen (Keijbets, 2008). In 2002, the Swedish National Food Administration reported alarmingly high levels of acrylamide in carbohydrate-rich heated foods (products from potato tubers, wheat flour, and coffee beans) (Tareke et al. 2002). While the potential carcinogeneticity of low levels of acrylamide in humans is still being investigated, (Pelucchi et al. 2003; Granath and Tomqvist, 2003), a great deal of research has focused on understanding the mechanism(s) of acrylamide formation in food as well as elimination/reduction strategies to minimize possible human health risk. In 2005 (Food Navigator report) several lawsuits were filed by the state of California against major food companies regarding acrylamide levels in potato-processed foods. As a result, several food companies agreed to substantially reduce the acrylamide levels in fried potato products over the following 3-5 years (San Francisco Chronicle article, 2008). However, the mechanisms regulating sugar accumulation in the cold remain poorly understood (Keijbets, 2008; Sowokinos, 2007), and a need remains for methods to reduce acrylamide levels in fried potato products.

Potato Carbohydrate Metabolism

Carbohydrate metabolism is complex in the potato (Sowokinos, 2007) and is thought to be a quantitative genetic trait (Menendez et al., 2002). The actual concentration of free sugar in potatoes involves the interaction of several pathways of carbohydrate metabolism, including starch synthesis/degradation, glycolysis, respiration and sweetening. These pathways are controlled at many levels, including hormonal, membrane structure and function, compartmentalization and concentration of enzymes, key ions, and substrate; and of course, enzyme expression levels and activity (Sowokinos, 2007).

Sucrose is synthesized from chloroplast-derived trosephosphate in a source leaf. After entering the apoplastic space around the phloem, a sucrose proton symporter actively takes the sucrose into the phloem. In a sink tissue, such as a tuber, sucrose is symplastically unloaded and/or released into the apoplast. From there, it can either be taken up by a sucrose proton symporter, or hydrolyzed by cell wall invertase to glucose and fructose. Within the sink cells, sucrose can either (1) be converted by sucrose synthase to uridine diphosphoglucose (UDPG) and fructose, or (2) hydrolyzed by a cytosolic invertase. After entering vacuoles, sucrose can also be split into fructose and glucose by vacuolar invertase. Hexokinases phosphorylate the simple sugars, resulting in hexoses that can enter respiration. In the sink tissue, cell wall invertase and vacuolar invertase can be regulated post-translationally by inhibitors of β-fructocidases (Rausch and Greiner, 2004).

A holy grail in industrial agriculture pertaining to potatoes, especially those to be processed into crisps, chips, and French fries, has been to control cold storage-induced sweetening of potatoes, a long-felt need for the potato processing industry (Keijbets, 2008). Solving the problem of cold-induced sweetening is particularly difficult because sugar content is affected by a multitude of factors, including (1) starch synthesis, (2) starch breakdown, (3) glycolysis, (4) mitochondrial respiration (in which the tuber is rich); and (5) hexogensis (Dale and Bradshaw, 2003). The importance of this goal is more easily understood when the statistics surrounding potato processing is understood. About 30 million metric tons of potatoes are converted into consumer products (crisps, chips, French fries, etc.) (Keijbets, 2008). While representing 10% of the global crop, processed potato products consume every 1-2 of every 3 potatoes produced in the developed countries of the world (Keijbets, 2008). Potatoes have begun to be important players even in China, which now is home to two modern French fry plants, twenty potato chip plants, and three potato flake plants (Keijbets, 2008). Add in potato starch processing, and China used 1.26 million metric tons of potatoes in 2006 (Keijbets, 2008). India is also ramping up (Keijbets, 2008). Overall, about 30 million metric tons of potatoes were used in processed potato products in 2006 (Keijbets, 2008).

Even though a potato molecular-function map for carbohydrate metabolism and transport was published in 2001-2002 (Chen et al., 2001; Menendez et al., 2002), the problem of cold storage-induced sweetening has been inadequately addressed. Several attempts demonstrate the tuber's resistance to manipulation concerning inhibition of cold storage-induced sweetening illustrates the challenge.

Zrenner et al. (1996) transformed potatoes with cold-inducible soluble acid invertase cDNA in the antisense orientation and under control of the constitutive 35S cauliflower mosaic virus promotor (Zrenner et al., 1996). Analysis of the harvested and cold-stored tubers showed that inhibition of the soluble acid invertase activity led to decreased hexose and increased sucrose content compared with controls. The hexose/sucrose ratio decreased with decreasing invertase activities, but Zrenner et al. observed that the total amount of soluble sugars did not significantly change. From these data, Zrenner et al. concluded that invertases do not control the total amount of soluble sugars in cold-stored potato tubers but are involved in the regulation of the ratio of hexose to sucrose (Zrenner et al., 1996).

Greiner et al. (1999) also had mixed results (Greiner et al., 1999). Greiner et al. transformed potato with cDNA encoding a putative vacuolar homolog of a tobacco cell wall invertase inhibitor operably linked to a CaMV 35S promoter. In transgenic tubers, cold-induced hexose accumulation was reduced by up to 75%, without any effect on potato tuber yield. Processing quality of tubers was improved without changing starch quantity or quality (Greiner et al., 1999), but Greiner et al. were only able to partially quell invertase activity.

### SUMMARY OF THE INVENTION

In a first aspect, the invention is directed to an isolated polynucleotide comprising a nucleic acid sequence having at least 90%-99% nucleic acid sequence identity with a sequence selected from the group consisting of SEQ ID NOs: 5, 6, 9, 10, 11, 23 and 24. In a related aspect, the present invention is related to a fragment of at least 20 contiguous nucleotides of a sequence having at least 90% sequence identity to SEQ ID NO:4. The invention is also directed to RNAi vectors comprising these polynucleotides, and transgenic plants containing these polynucleotides and vectors. Transgenic plants include those from the genus Solanum, such as potato (Solanum tuberosum) as well as sweet potato, yams and Cassaya.

In a second aspect, the invention is directed to an isolated polynucleotide comprising a nucleic acid sequence selected from the group consisting of SEQ ID NOs: 5, 6, 9, 10, 11, 23 and 24.

In the first two aspects, the invention is also directed to RNAi vectors comprising the polynucleotides of these first two aspects. The invention is also directed to RNAi vectors comprising these polynucleotides, and transgenic plants containing these polynucleotides and vectors. Transgenic plants include those from the genus Solanum, such as potato (Solanum tuberosum) as well as sweet potato, yams and Cassaya. The invention also includes edible products from such transgenic plants, such as potatoes, as well as their processed form, including for potatoes, crisps, potato chips, French fries, potato sticks and shoestring potatoes. For sweet potatoes, such processed forms include, sweet potatoes, crisp, chips and fries. The expression of a vacuolar invertase (VI) gene is decreased by at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or greater in transgenic plants when compared to a non-transformed plant or other control.

In a third aspect, the invention is directed to methods for silencing vacuolar invertase in a transgenic potato plant or transgenic sweet potato plant comprising decreasing the level of VI activity compared to its level in a control, non-transgenic potato plant or non-transgenic sweet potato plant by reducing the level of an mRNA in the transgenic potato plant or transgenic sweet potato plant, wherein the mRNA is encoded by a polynucleotide having at least 90% sequence identity to a nucleic acid sequence of SEQ ID NO:4, and by expression of an RNAi construct comprising a fragment of at least 20 contiguous nucleotides of a sequence having at least 90% sequence identity to SEQ ID NO:4.

In a fourth aspect, the invention is directed to methods for silencing vacuolar invertase in a transgenic potato plant or transgenic sweet potato plant comprising decreasing the level of VI activity compared to its level in a control, non-transgenic potato plant or control, non-transgenic sweet potato plant by reducing the level of an mRNA in the transgenic potato plant or transgenic sweet potato plant, wherein the mRNA is encoded by a polynucleotide having at least 90% sequence identity to a nucleic acid sequence of SEQ ID NO:4, and by expression of an RNAi construct comprising a polynucleotide having at least 90%-99% sequence identity to a polynucleotide selected from the group consisting of SEQ ID NOs:5, 6, 9, 10, 11, 23 and 24. The RNAi construct can also comprise a polynucleotide selected from the group consisting of SEQ ID NOs: 5, 6, 9, 10, 11, 23 and 24.

In the third and fourth aspects, the invention can further comprise a step of screening the transgenic plants for a reduction in VI activity by comparing the VI activity in the transgenic plant to a control plant, such as a non-transgenic plant, or a transgenic plant having an empty vector. These methods can also further comprise a step of screening potatoes or sweet potatoes produced by transgenic plants by comparing the transgenic potato or transgenic sweet potato with a control potato or control sweet potato for cold storage-induced sweetening. Such screening can include assaying chip color after frying. Examples of assays that can be used include visual color rating, such as the one provided herein in Table 6. Chip color can be visually determined using the Potato Chip Color Reference Standards developed by Potato Chip Institute International, Cleveland, Ohio (Douches and Freyer, 1994; Reeves, 1982).

Also in the third and fourth aspects, the RNAi vector can be introduced into plants using Agrobacterium tumefaciens. The RNAi vector can comprise, for example, a pHELLSGATE vector, such as pHELLSGATE2 or pHELLSGATE8. Plants amenable to the methods of the invention include those from the genus Solanum, such as potato (Solanum tuberosum) as well as sweet potato, yams and Cassaya.

In a fifth aspect, the invention is directed to kits comprising an RNAi construct comprising a fragment of at least 20 contiguous nucleotides of a sequence having at least 90% sequence identity to SEQ ID NO:4, and instructions for use. For example, the RNAi construct can comprise a polynucleotide having at least 90%-99% sequence identity to a polynucleotide selected from the group consisting of SEQ ID NOs: 5, 6, 9, 10, 11, 23 and 24. The RNAi construct can also comprise a polynucleotide selected from the group consisting of SEQ ID NOs: 5, 6, 9, 10, 11, 23 and 24.

In a sixth aspect, the invention is directed to methods for controlling the accumulation of reducing sugars in a potato plant or sweet potato plant during cold storage. The method comprises the steps of decreasing a level of vacuolar invertase activity in the potato plant or sweet potato plant relative to a control potato plant or sweet potato plant by introducing to the potato plant an RNAi construct comprising a fragment of at least 20 contiguous nucleotides of a sequence having at least 90% sequence identity to SEQ ID NO:4, and maintaining the plant under conditions sufficient for expression of the RNAi construct thereby decreasing the level of an mRNA that is encoded by a polynucleotide having at least 90% sequence identity to a nucleic acid sequence of SEQ ID NO:4. This method can further comprise assaying the color of a potato product or sweet potato product from a potato or sweet potato of the plant after heat processing the potato or sweet potato. Alternatively, the method can involve assaying the color of the potato product or sweet potato product by comparing the product color with the color of a control potato product or control sweet potato product from a control potato plant. The above method can further comprise heat processing the potato into a crisp, chip, French fry, potato stick, shoestring potato or other edible potato product or sweet potato into a crisp, chip, fry or other sweet potato product.

In the above method the RNAi construct comprises a polynucleotide having at least 90% sequence identity to a polynucleotide selected from the group consisting of SEQ ID NOs: 5, 6, 9, 10, 11, 23 and 24. Alternatively, the RNAi construct comprises a polynucleotide having at least 95% sequence identity to a polynucleotide selected from the group consisting of SEQ ID NOs: 5, 6, 9, 10, 11, 23 and 24. Still further alternatively, the RNAi construct comprises a polynucleotide having at least 98% sequence identity to a polynucleotide selected from the group consisting of SEQ ID NOs: 5, 6, 9, 10, 11, 23 and 24.

Still alternatively, the RNAi construct comprises a polynucleotide selected from the group consisting of SEQ ID NOs: 5, 6, 9, 10, 11, 23 and 24.

In this method, the RNAi vector can be introduced into plants using Agrobacterium tumefaciens. The RNAi vector can comprise, for example, a pHELLSGATE vector, such as pHELLSGATE2 or pHELLSGATE8. Plants amenable to the methods of the invention include those from the genus Solanum, such as potato (Solanum tuberosum) as well as sweet potato, yams and Cassaya.

In a seventh aspect, the invention is directed to a method for controlling acrylamide formation during heat processing of a potato or sweet potato from a potato plant or sweet potato plant. The method comprises the steps of decreasing a level of vacuolar invertase activity in the potato plant or sweet potato plant relative to a control potato plant or sweet potato plant by introducing to the potato plant or sweet potato plant an RNAi construct comprising a fragment of at least 20 contiguous nucleotides of a sequence having at least 90% sequence identity to SEQ ID NO:4, and maintaining the plant under conditions sufficient for expression of the RNAi construct thereby decreasing the level of an mRNA that is encoded by a polynucleotide having at least 90% sequence identity to a nucleic acid sequence of SEQ ID NO:4.

This method can further comprise assaying the level of acrylamide in a heat processed potato product or sweet potato product of a potato from a potato plant or sweet potato from a sweet potato product produced by the above method.

The assaying of the level of acrylamide in the potato product or sweet potato product can further comprise comparing the acrylamide level of a potato product or sweet potato product derived from a potato from a potato plant or sweet potato from a sweet potato product produced by the above method with an acrylamide level in a control potato product from a control potato plant or a control sweet potato product from a control sweet potato plant. When assayed, potato products or sweet potato products derived from a potato from a potato plant or a sweet potato from a sweet potato plant produced by the above method and which potato or sweet potato has been subjected to cold storage for a period of at least 2 hours can exhibit at least a 5 fold reduction, at least a 6 fold reduction, at least a 7 fold reduction, at least a 8 fold reduction, at least a 9 fold reduction, at least a 10 fold reduction, at least a 11 fold reduction, at least a 12 fold reduction, at least a 13 fold reduction, at least a 14 fold reduction, at least a 15 fold reduction, at least a 20 fold reduction, at least a 25 fold reduction, at least a 30 fold reduction, at least a 35 fold reduction, at least a 40 fold reduction, at least aa 45 fold reduction, at least a 50 fold reduction, at least a 55 fold reduction, at least a 60 fold reduction, at least a 65 fold reduction, at least a 70 fold reduction, at least a 75 fold reduction, at least a 80 fold reduction, at least a 85 fold reduction, at least a 90 fold reduction, at least a 95 fold reduction, at least a 100 fold reduction, at least a 150 fold reduction, at least a 200 fold reduction, at least a 250 fold reduction, at least a 300 fold reduction, at least a 350 fold reduction, at least a 400 fold reduction, at least a 450 fold reduction or at least a 500 fold reduction in the level of acrylamide when compared to a potato product from a control potato plant. More specifically, the potato or sweet potato has been subjected to cold storage for a period of at least three hours, at least four hours, at least five hours, at least six hours, at least eight hours, at least ten hours, at least 12 hours, at least 18 hours, at least 24 hours, at least 30 hours, at least 36 hours or longer. Alternatively, when assayed, potato products or sweet potato products derived from a potato from a potato plant or a sweet potato from a sweet potato plant produced by the above method and which potato or sweet potato has been subjected to or stored at room temperature conditions (19.5° to 25.5° C./67.1° F. to 77.9° F.) can exhibit at least a 1 fold reduction, at least a 2 fold reduction, at least a 3 fold reduction, at least a 4 fold reduction, at least a 5 fold reduction, at least a 6 fold reduction, at least a 7 fold reduction, at least a 8 fold reduction, at least a 9 fold reduction, at least a 10 fold reduction, at least a 11 fold reduction, at least a 12 fold reduction, at least a 13 fold reduction, at least a 14 fold reduction or at least a 15 fold reduction in the level of acrylamide when compared to a potato product from a control potato plant.

Alternatively, the potato products derived from a potato from a potato plant or the sweet potato products derived from a sweet potato from a sweet potato plant produced by the above method and which potato or sweet potato has been subjected to cold storage for a period of at least 2 hours when assayed exhibit a 5 to 500 fold reduction, a 5 to 450 fold reduction, a 5 to 400 fold reduction, a 5 to 400 fold reduction, a 5 to 350 fold reduction, a 5 to 300 fold reduction, a 5 to 250 fold reduction, a 5 to 200 fold reduction, a 5 to 150 fold reduction, a 5 to 100 fold reduction, a 5 to 95 fold reduction, a 5 to 90 fold reduction, a 5 to 85 fold reduction, a 5 to 80 fold reduction, a 5 to 75 fold reduction, a 5 to 70 fold reduction, a 5 to 65 fold reduction, a 5 to 60 fold reduction, a 5 to 55 fold reduction, a 5 to 50 fold reduction, a 5 to 45 fold reduction, a 5 to 40 fold reduction, a 5 to 35 fold reduction, a 5 to 30 fold reduction, a 5 to 25 fold reduction, a 5 to 20 fold reduction, a 5 to 15 fold reduction, a 5 to 10 fold reduction, a 10 to 500 fold reduction, a 10 to 450 fold reduction, a 10 to 400 fold reduction, a 10 to 400 fold reduction, a 10 to 350 fold reduction, a 10 to 300 fold reduction, a 10 to 250 fold reduction, a 10 to 200 fold reduction, a 10 to 150 fold reduction, a 10 to 100 fold reduction, a 10 to 95 fold reduction, a 10 to 90 fold reduction, a 10 to 85 fold reduction, a 10 to 80 fold reduction, a 10 to 75 fold reduction, a 10 to 70 fold reduction, a 10 to 65 fold reduction, a 10 to 60 fold reduction, a 10 to 55 fold reduction, a 10 to 50 fold reduction, a 10 to 45 fold reduction, a 10 to 40 fold reduction, a 10 to 35 fold reduction, a 10 to 30 fold reduction, a 10 to 25 fold reduction, a 10 to 20 fold reduction or a 10 to 15 fold reduction in the level of acrylamide when compared to a potato product from a control potato plant or a sweet potato product from a control sweet potato plant. More specifically, the potato or sweet potato has been subjected to cold storage for a period of at least three hours, at least four hours, at least five hours, at least six hours, at least eight hours, at least ten hours, at least 12 hours, at least 18 hours, at least 24 hours, at least 30 hours, at least 36 hours or longer. Alternatively, when assayed, potato products or sweet potato products derived from a potato from a potato plant or a sweet potato from a sweet potato plant produced by the above method and which potato or sweet potato has been subjected to or stored at room temperature conditions can exhibit a reduction of at least a 1 to 15 fold reduction, a 2 to 15 fold, a 3 to 15 fold, a 4 to 15 fold, a 5 to 15 fold, a 1 to 14 fold, a 2 to 14 fold, a 3 to 14 fold, a 4 to 14 fold a 5 to 14 fold, a 1 to 13 fold, a 2 to 13 fold, a 3 to 13 fold, a 4 to 13 fold a 5 to 15 fold, a 1 to 12 fold, a 2 to 12 fold, a 3 to 12 fold, a 4 to 12 fold, a 5 to 12 fold, a 1 to 11 fold, a 2 to 11 fold, a 3 to 11 fold, a 4 to 11 fold, a 5 to 11 fold, a 1 to 10 fold, a 2 to 10 fold, a 3 to 10 fold, a 4 to 10 fold or a 5 to 10 fold in the level of acrylamide when compared to a potato product from a control potato plant.

Still further alternatively, the potato products derived from a potato from a potato plant or the sweet potato products derived from a sweet potato from a sweet potato plant produced by the above method and which potato or sweet potato has been subjected to cold storage for a period of at least 2 hours when assayed exhibit levels of acrylamide 25% to 75% less, 25% to 70% less, 25% to 65% less, 25% to 60% less, 25% to 55% less, 25% to 55% less, 25% to 50% less, 25% to 45% less, 25% to 40% less, 25 to 35% less, 30% to 75% less, 30% to 70% less, 30% to 65% less, 30% to 60% less, 30% to 55% less, 30% to 55% less, 30% to 50% less, 30% to 45% less, 25% to 40% less, 30% to 35% less, 35% to 75% less, 35% to 70% less, 35% to 65% less, 35% to 60% less, 35% to 55% less, 35% to 55% less, 35% to 50% less, 35% to 45% less, 35% to 40% less, 40% to 75% less, 40% to 70% less, 40% to 65% less, 40% to 60% less, 40% to 55% less, 40% to 55% less, 40% to 50% less, 40% to 45% less, 45% to 75% less, 45% to 70% less, 45% to 65% less, 45% to 60% less, 45% to 55% less, 45% to 55% less, 45% to 50%, 50% to 75% less, 50% to 70% less, 50% to 65% less, 50% to 60% less or 50% to 55% less, when compared to a potato product from a control potato plant or a sweet potato product from a control sweet potato plant. More specifically, the potato or sweet potato has been subjected to cold storage for a period of at least three hours, at least four hours, at least five hours, at least six hours, at least eight hours, at least ten hours, at least 12 hours, at least 18 hours, at least 24 hours, at least 30 hours, at least 36 hours or longer. Alternatively, when assayed, potato products or sweet potato products derived from a potato from a potato plant or a sweet potato from a sweet potato plant produced by the above method and which potato or sweet potato has been subjected to or stored at room temperature conditions can exhibit levels of acrylamide 25% to 75% less, 25% to 70% less, 25% to 65% less, 25% to 60% less, 25% to 55% less, 25% to 55% less, 25% to 50% less, 25% to 45% less, 25% to 40% less, 25 to 35% less, 30% to 75% less, 30% to 70% less, 30% to 65% less, 30% to 60% less, 30% to 55% less, 30% to 55% less, 30% to 50% less, 30% to 45% less, 25% to 40% less, 30% to 35% less, 35% to 75% less, 35% to 70% less, 35% to 65% less, 35% to 60% less, 35% to 55% less, 35% to 55% less, 35% to 50% less, 35% to 45% less, 35% to 40% less, 40% to 75% less, 40% to 70% less, 40% to 65% less, 40% to 60% less, 40% to 55% less, 40% to 55% less, 40% to 50% less, 40% to 45% less, 45% to 75% less, 45% to 70% less, 45% to 65% less, 45% to 60% less, 45% to 55% less, 45% to 55% less, 45% to 50%, 50% to 75% less, 50% to 70% less, 50% to 65% less, 50% to 60% less or 50% to 55% less, when compared to a potato product from a control potato plant or a sweet potato product from a control sweet potato plant.

Additionally, it is also believed that when assayed as described above, potato products derived from a potato from a potato plant or sweet potato products derived from a sweet potato from a sweet potato plant produced by the above method and which potato or sweet potato has been subjected to cold storage for a period of at least 2 hours will exhibit levels of acrylamide less than 500 ppb (mg/Kg), less than 400 ppb (mg/Kg), less then 300 ppb (mg/Kg), less then 200 ppb (mg/Kg) or less than less then 100 ppb (mg/Kg). Alternatively, when assayed, the potato products derived from a potato from a potato plant produced by the above method will exhibit levels of acrylamide between about 90 ppb (mg/Kg) to about 500 ppb (mg/Kg), about 100 ppb (mg/Kg) to about 500 ppb (mg/Kg), about 200 ppb (mg/Kg) to about 500 ppb (mg/Kg), about 250 ppb (mg/Kg) to about 500 ppb (mg/Kg), about 100 ppb (mg/Kg) to about 300 ppb (mg/Kg), about 100 ppb (mg/Kg) to about 250 ppb (mg/Kg), about 200 ppb (mg/Kg) to about 300 ppb (mg/Kg), about 250 ppb (mg/Kg) to about 300 ppb (mg/Kg), about 300 ppb (mg/Kg) to about 500 ppb (mg/Kg), or about 400 ppb (mg/Kg) to about 500 ppb (mg/Kg). More specifically, the potato or sweet potato has been subjected to cold storage for a period of at least three hours, at least four hours, at least five hours, at least six hours, at least eight hours, at least ten hours, at least 12 hours, at least 18 hours, at least 24 hours, at least 30 hours, at least 36 hours or longer. Additionally, it is also believed that when assayed as described above, potato products derived from a potato from a potato plant or sweet potato products derived from a sweet potato from a sweet potato plant produced by the above method and which potato or sweet potato has been subjected to or stored at room temperature conditions can exhibit levels of acrylamide less than 1100 ppb (mg/Kg), 1000 ppb (mg/Kg), less than 900 ppb (mg/Kg), less then 800 ppb (mg/Kg), less then 700 ppb (mg/Kg), less than less then 600 ppb (mg/Kg), or less than 500 ppb (mg/Kg). Alternatively, when assayed, the potato products derived from a potato from a potato plant produced by the above method will exhibit levels of acrylamide between about 400 ppb (mg/Kg) to about 1100 ppb (mg/Kg), about 400 ppb (mg/Kg) to about 1000 ppb (mg/Kg), about 400 ppb (mg/Kg) to about 900 ppb (mg/Kg), about 400 ppb (mg/Kg) to about 800 ppb (mg/Kg), about 400 ppb (mg/Kg) to about 700 ppb (mg/Kg), about 500 ppb (mg/Kg) to about 1100 ppb (mg/Kg), about 500 ppb (mg/Kg) to about 1000 ppb (mg/Kg), about 500 ppb (mg/Kg) to about 900 ppb (mg/Kg), about 500 ppb (mg/Kg) to about 800 ppb (mg/Kg) or about 500 ppb (mg/Kg) to about 750 ppb (mg/Kg).

The above method can further comprise heat processing the potato into a crisp, chip, French fry potato stick, shoestring potato or other edible potato product or the sweet potato into a crisp, chip, fry or other sweet potato product.

In the above method the RNAi construct comprises a polynucleotide having at least 90% sequence identity to a polynucleotide selected from the group consisting of SEQ ID NOs: 5, 6, 9, 10, 11, 23 and 24. Alternatively, the RNAi construct comprises a polynucleotide having at least 95% sequence identity to a polynucleotide selected from the group consisting of SEQ ID NOs: 5, 6, 9, 10, 11, 23 and 24. Still further alternatively, the RNAi construct comprises a polynucleotide having at least 98% sequence identity to a polynucleotide selected from the group consisting of SEQ ID NOs: 5, 6, 9, 10, 11, 23 and 24.

Still alternatively, the RNAi construct comprises a polynucleotide selected from the group consisting of SEQ ID NOs: 5, 6, 9, 10, 11, 23 and 24.

In this method, the RNAi vector can be introduced into plants using Agrobacterium tumefaciens. The RNAi vector can comprise, for example, a pHELLSGATE vector, such as pHELLSGATE2 or pHELLSGATE8. Plants amenable to the methods of the invention include those from the genus Solanum, such as potato (Solanum tuberosum) as well as sweet potato, yams and Cassaya.

### BRIEF DESCRIPTION OF SEVERAL VIEWS OF THE DRAWING

FIG. 1 shows a Northern blot analyzing several transgenic potato lines containing an RNAi construct targeting VI. (A) A Northern blot showing the mRNA expression patterns of VI gene among potato lines. C=Non-transformed potato plant (control), lines 1-15 represent samples from 15 independent VI-RNAi transgenic plants. VI mRNA levels were reduced as low as 95-99% in samples 1, 2, 5 and 13, where as various levels of silencing patterns was noticed in remaining RNAi lines. (B) A gel loading control of RNA samples confirming the loading patterns and the integrity of RNA. The gel picture was taken under UV after staining with ethidium bromide for 10 minutes.

FIG. 2 shows a phenotypic analysis of exemplary transgenic potato lines containing an RNAi construct targeting VI. (A) No abnormal phenotypes were observed among the VI silenced transgenic Katandin plants compared to non-transgenic Katandin (control) during greenhouse experiments. The pictures were taken on plants 50 days old. (B) Tubers harvested from each of the lines. No abnormal tuber phenotypes were observed, and no significant differences (P<0.05) were observed in tuber yield between VI silenced lines and controls.

FIG. 3 shows chipping experiments that assay for the Maillard reaction by chip color. Top panel shows chips obtained from tuber samples taken from one representative VI silenced RNAi line (#1) (˜99% silenced) stored at room temperature (20° C.) for 60 days and at cold storage (4° C.) for a period of 14 days, 60 days, 90 and 180 days. Bottom panel shows chips obtained from tuber samples taken from non-transformed (Katandin-control) tubers stored at room temperature (20° C.) for 60 days and at cold storage (4° C.) for a period of 14, 60, 90 and 180 days. A visual potato chip color rating of 3.0 was scored to chips sampled from tubers of room temperature stored, both control and RNAi line. Strikingly, a chip score of 3.0 was given to chips sampled from 14, 60, 90 and 180 day cold-stored RNAi line tubers. However, a chip score of 6.0 was scored to chips sampled from cold stored control line at 14 days and 8.0 for all chips sampled from 60, 90 and 180 days cold storage taken directly. The chip scale represents 1 (light) to 10 (dark). A visual potato chip color rating is provided in Table 6.

FIG. 4 shows the correlation of chip color with the amount of VInv transcript. All the chips were obtained from tuber samples taken at 60 day storage either at 20° C. or at 4° C. (direct chipping from cold storage). Representative tuber samples were collected from VI RNAi lines representing various levels of transcripts as described in Table 6. RNAi line #10 has no VI transcript reduction and produced a poor chip score of 8.0 at 60 day chipping stored at 4° C. RNAi line #1 has 99% VI transcript reduction and produced a good chip score of 3.0 at 60 day chipping (tubers stored at 4° C.). RNAi line #3 has ˜90% VI transcript reduction and produced a medium chip score of 5.5 at 60 day chipping (tubers stored at 4° C.). RNAi line #5 has 80% VI transcript reduction and produced a medium chip score of 5.0 at 60 day chipping (tubers stored at 4° C.). RNAi lines #6, 8 have 60% and 20% transcript reductions respectively. Both these lines produced poor chip scores of 7.0 at 60 day chipping (tubers stored at 4° C.).

FIG. 5 is a bar graph of acrylamide levels in potato chips derived from VI silencing lines. Acrylamide analysis was performed on chips obtained from tuber samples of three representative VI silenced RNAi lines (using RNAi #1, 2, 3) and Katandin (control), cold-stored at 4° C. for 14 days. Acrylamide levels are shown as ppb (mg/kg) and represents the mean of two independent measurements including standard deviation. Asterisks indicate significant differences of RNAi lines from Katandin control line (P<0.05).

FIG. 6 is a bar graph of acrylamide levels in potato chips derived from VI silencing lines. Acrylamide analysis was performed on chips obtained from tuber samples of three representative VI silenced RNAi lines (RNAi #1, 2, 3) and Katandin (control), cold-stored at 4° C. for 180 days. Acrylamide levels are shown as ppb (mg/kg) and represents the mean of two independent measurements including standard deviation. Asterisks indicate significant differences of RNAi lines from Katandin control line (P<0.05).

FIG. 7 is a comparative acrylamide patterns among tubers stored at RT or at 4° C. for 14 days and for 180 days. Acrylamide levels among RNAi lines (#1, 2, 3) showed only slight changes between RT stored and 4° C. for 14 days and 180 days compared to controls. However, acrylamide levels increased several fold higher among tubers obtained from Katandin control lines when stored at 4° C. for 14 days or 180 days. Acrylamide levels are shown as ppb (mg/Kg).

FIG. 8 shows the field evaluations of VI-RNAi lines of the present invention grown in two locations in Wisconsin compared control (C) and empty vector lines (EV) as described in Example 9. More specifically, tuber yield comparisons among field grown control and VI-RNAi lines. The mean total yield (g) per plant (n=9) among control and empty vector transformed plants were 2123±253 and 2120±392 respectively. The mean total yield (g) among three independent VI-RNAi lines #2, #3 and #1 were 1963±233, 1961±329 and 1767±166 respectively. Fisher's Least Significant Difference (LSD) test as a comparison of mean total yields revealed no significant differences (alpha of 0.05) among controls (control and empty vector) and #2 and #3. However, LSD test at an alpha of 0.05 revealed significant difference among controls and #1 line. Significant differences from controls (P<0.05) are indicated with an asterisk.

FIG. 9 shows specific gravity measurements of tubers harvested field grown VI-RNAi lines of the present invention (#2, #3, #1) grown in two locations in Wisconsin compared control (C) and empty vector lines (EV) as described in Example 9. This figure shows that the VI-RNAi lines (#2, #3, #1) showed specific gravity measurements that were consistent (p<0.05) compared to the control and empty vector lines.

FIG. 10 shows the results of chipping experiments that assay for Maillard reaction by chip color on tubers obtained from field grown VI-RNAi lines of the present invention grown in two locations in Wisconsin. The top panel shows chips obtained from field grown tuber samples taken from ten representative VI silenced RNAi lines of #1, #2, #3 (˜99% silenced) and controls stored at cold storage (4° C.) for a period of 14 days. The bottom panel shows chips obtained from field grown tuber samples taken from ten representative VI silenced RNAi lines of #1, #2, #3 (˜99% silenced) and controls stored at room temperature (RT) for a period of 14 days.

FIG. 11 shows further results of chipping experiments on tubers obtained from field grown VI-RNAi lines of the present invention grown in two locations in Wisconsin. Specifically, this figure shows chip color of field grown tubers subjected to either cold storage for 14 days at 4° C. or stored at room temperature (RT) for 14 days. 50 independent chips were sliced from 10 different tubers from each of the lines (#1, #2, #3 and control) and Hunter value measurements were taken. The horizontal dash bar represents the lower limit of commercially acceptable Hunter color score. Hunter ratings of ≧50 are generally acceptable scores.

FIG. 12 this figure shows acrylamide levels in potato chips derived from field grown VI silencing lines. Acrylamide analysis was performed on chips obtained from field grown tuber samples of three representative VI silenced RNAi lines (RNAi #1, 2, 3) and Katandin (control), cold stored at 4° C. for 14 days. Acrylamide levels are shown as ppb (mg/kg) and represent the mean of three independent measurements including standard deviation. Asterisks indicate significant differences of RNAi lines from Katandin control line (P<0.05). Chips processed from tubers stored at 4° C. showed lower acrylamide levels than chips from tubers stored at 20° C. for two of the three lines.

FIG. 13 shows acrylamide levels in potato chips derived from greenhouse grown VI silencing lines. Acrylamide analysis was performed on chips obtained from greenhouse grown tuber samples of three representative VI silenced RNAi lines (RNAi #1, 2, 3) and Katandin (control), cold stored at 4° C. for 14 days. Acrylamide levels are shown as ppb (mg/kg) and represent the mean of three independent measurements including standard deviation. Asterisks indicate significant differences of RNAi lines from Katandin control line (P<0.05). Chips processed from tubers stored at 4° C. showed lower acrylamide levels than chips from tubers stored at 20° C. for all the three lines.

### DETAILED DESCRIPTION

The present invention surprisingly and simply solves conclusively the cold-storage induced sweetening in potatoes, thus finally providing a final, satisfactory solution to the long-felt need of eliminating the complications from storage at low temperatures (2-12° C.).

The inventors were surprised that silencing the vacuolar invertase (VI) gene using an RNA-interference (RNAi) approach was alone sufficient, especially given the complex nature of carbohydrate metabolism in potatoes (Menendez et al., 2002; Sowokinos, 2007). The inventors have developed several lines of potatoes in which the VI gene is silenced partially or completely in the entire potato plant. These lines showed variable invertase RNA levels compared with the control plants. Not only was it surprising to solve the cold storage-induced sweetening problem so simply, the inventors observed no deleterious side effects: no phenotypic abnormalities or other negative effects were observed, including tuber size, shape and average weight (tuber yield). Chipping experiments performed on the most silent lines stored at 39° F. (4° C.) for two months, 3 months and prolonged 6 months produced dramatic, light-colored, industry acceptable potato chips. Such chipping experiments involve assaying chip color (for the Maillard reaction) after frying. Examples of assays that can be used include visual color rating, such as the one provided herein in Table 6. Chip color can be visually determined using the Potato Chip Color Reference Standards developed by Potato Chip Institute International, Cleveland, Ohio (Douches and Freyer, 1994; Reeves, 1982). These results therefore not only demonstrate that cold storage-induced sweetening can be surprisingly simply solved, but also cause a paradigm shift in potato carbohydrate metabolism and cold storage-induced sweetening in the potato. No longer can CIS in potato be considered to be a complex, quantitative trait (Menendez et al., 2002), but a simple trait that can be manipulated by a single gene, the vacuolar acid invertase gene.

The invention is accomplished by decreasing the level of VI activity compared to its level in a control, non-transgenic potato plant by reducing the level of an mRNA in the transgenic potato plant, wherein the mRNA is encoded by a polynucleotide having at least 90% sequence identity to a nucleic acid sequence of SEQ ID NO:4, and by expression of an RNAi construct comprising a fragment of at least 20 contiguous nucleotides of a sequence having at least 90% sequence identity to SEQ ID NO:4. For example, the invention can be accomplished by expressing an RNAi construct comprising a polynucleotide having at least 90%-99% sequence identity to a polynucleotide selected from the group consisting of SEQ ID NOs: 5, 6, 9, 10, 11, 23 and 24 in a plant, such as a potato plant. The RNAi construct can also comprise a polynucleotide selected from the group consisting of SEQ ID NOs: 5, 6, 9, 10, 11, 23 and 24.

The methods of the invention can be easily accomplished using conventional transgenic techniques and recombinant DNA technologies.

### DEFINITIONS

“About” refers to a plus or minus about ten percent (10%) of a recited value.

“Cold storage” as used herein refers to the storage of a potato or sweet potato at a temperature of 12° C. or less. Alternatively, “cold storage” refers to a range of a temperature of from 2° C. to 12° C. Examples of “cold storage” temperatures for potato and sweet potato are temperatures from 2° C. to 4° C. or 8° C. to 10° C. Cold storage can occur for a period of at a period for at least 2 hours. More specifically, cold storage can occur for a period of at least three hours, at least four hours, at least five hours, at least six hours, at least eight hours, at least ten hours, at least 12 hours, at least 18 hours, at least 24 hours, at least 30 hours, at least 36 hour or longer.

“Room temperature conditions” or “room temperature” as used interchangeably herein means a temperature from between 18° to 26° C. More specifically, room temperature conditions or room temperature can be a temperature from 19.5° C. to 25.5° C.

“Potato product” or “Edible potato product” as used interchangeably herein refers to foodstuffs derived from potatoes for consumption, such as, but not limited to, crisps, potato chips, shoestrings (also known as potato sticks), French fries, potato sticks and shoestring potatoes (Shoestring potatoes are extremely thin (namely, 2-3 mm) versions of regular French fries, but are fried in the manner of regular salted potato chips).

“Heat processing” as used herein refers to heating a potato product or sweet potato product in oil (such as corn oil, olive oil, vegetable oil, peanut oil, canola oil) or fat at a temperature of from 160° F. to about 375° F., using routine techniques known in the art (such as traditional deep-frying, vacuum frying, oven-frying, kettle frying, etc.).

“Potato” as used herein refers to any varieties of Solanum tuberosum. Examples of varieties of Solanum tuberosum that can be in the present invention are Allegany, Atlantic, CalWhite, Cascade, Castile, Chipeta, Gemchip, Irish Cobbler, Freedom Russet, Itasca, Kanona, Katandin, Kennebec, La Chipper, MegaChip, Millennium Russet, Monona, Norchip, Norwis, Onaway, Ontario, Pike, Sebago, Shepody, Snowden, Superior, White Rose, Yukon Gold, Red Rounds, Chieftain, La Rouge, NorDonna, Norland, Red La Soda, Red Pontiac, Red Ruby, Sangre, Viking, Russets, BelRus, Centennial Russet, Century Russet, Frontier Russet, Goldrush, Hilite Russet, Krantz, Lemhi Russet, Nooksack, Norgold Russet, Norking Russet, Dakota Pearl, Ranger Russet, Ranger Russet Mews Release, Russet Burbank, Russet Norkotah, Russet Nugget Villetta Rose and White Pearl.

“Specifically hybridize” refers to the ability of a nucleic acid to bind detectably and specifically to a second nucleic acid. Polynucleotides specifically hybridize with target nucleic acid strands under hybridization and wash conditions that minimize appreciable amounts of detectable binding by non-specific nucleic acids.

A “targeting” sequence means a nucleic acid sequence of Solanum tuberosum VI sequence or complements thereof can silence a VI gene. Exemplary targeting sequences include SEQ ID NOs: 9-11 and 23-24. A target sequence can be selected that is more or less specific for a particular cultivar of Solanum tuberosum. For example, the targeting sequence can be specific to VI genes from the potato varieties of, for example, Allegany, Atlantic, CalWhite, Cascade, Castile, Chipeta, Gemchip, Irish Cobbler, Freedom Russet, Itasca, Kanona, Katandin, Kennebec, La Chipper, MegaChip, Millennium Russet, Monona, Norchip, Norwis, Onaway, Ontario, Pike, Sebago, Shepody, Snowden, Superior, White Rose, Yukon Gold, Red Rounds, Chieftain, La Rouge, NorDonna, Norland, Red La Soda, Red Pontiac, Red Ruby, Sangre, Viking, Russets, BelRus, Centennial Russet, Century Russet, Frontier Russet, Goldrush, Hilite Russet, Krantz, Lemhi Russet, Nooksack, Norgold Russet, Norking Russet, Dakota Pearl, Ranger Russet, *Ranger Russet Mews Release, Russet Burbank, Russet Norkotah, Russet Nugget Villetta Rose and White Pearl.

A “polynucleotide” is a nucleic acid polymer of ribonucleic acid (RNA), deoxyribonucleic acid (DNA), modified RNA or DNA, or RNA or DNA mimetics (such as, PNAs), and derivatives thereof, and homologues thereof. Thus, polynucleotides include polymers composed of naturally occurring nucleobases, sugars and covalent inter-nucleoside (backbone) linkages as well as polymers having non-naturally-occurring portions that function similarly. Such modified or substituted nucleic acid polymers are well known in the art and for the purposes of the present invention, are referred to as “analogues.” Oligonucleotides are generally short polynucleotides from about 10 to up to about 160 or 200 nucleotides.

“Solanum tuberosum VI (sequence variant polynucleotide” or “Solanum tuberosum VI sequence variant nucleic acid sequence” means a Solanum tuberosum VI sequence variant polynucleotide having at least about 60% nucleic acid sequence identity, more preferably at least about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% nucleic acid sequence identity and yet more preferably at least about 99% nucleic acid sequence identity with the nucleic acid sequence of. Variants do not encompass the native nucleotide sequence.

Ordinarily, Solanum tuberosum VI sequence variant polynucleotides are at least about 8 nucleotides in length, often at least about 9, 10, 11, 12, 13, 14, 15, 16, 17, 18, 19, 20, 21, 22, 23, 24, 25, 26, 27, 28, 29 30, 35, 40, 45, 50, 55, 60 nucleotides in length, or even about 75-200 nucleotides in length, or more.

“Percent (%) nucleic acid sequence identity” with respect to Solanum tuberosum VI sequence-nucleic acid sequences is defined as the percentage of nucleotides in a candidate sequence that are identical with the nucleotides in the Solanum tuberosum VI sequence of interest, after aligning the sequences and introducing gaps, if necessary, to achieve the maximum percent sequence identity. Alignment for purposes of determining % nucleic acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

When nucleotide sequences are aligned, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) can be calculated as follows:

% nucleic acid sequence identity=W/Z·100

where

W is the number of nucleotides cored as identical matches by the sequence alignment program's or algorithm's alignment of C and D

and

Z is the total number of nucleotides in D.

When the length of nucleic acid sequence C is not equal to the length of nucleic acid sequence D, the % nucleic acid sequence identity of C to D will not equal the % nucleic acid sequence identity of D to C.

“Consisting essentially of a polynucleotide having a % sequence identity” means that the polynucleotide does not substantially differ in length, but in sequence. Thus, a polynucleotide “A” consisting essentially of a polynucleotide having 80% sequence identity to a known sequence “B” of 100 nucleotides means that polynucleotide “A” is about 100 nts long, but up to 20 nts can vary from the “B” sequence. The polynucleotide sequence in question can be longer or shorter due to modification of the termini, such as, for example, the addition of 1-15 nucleotides to produce specific types of probes, primers and other molecular tools, etc., such as the case of when substantially non-identical sequences are added to create intended secondary structures. Such non-identical nucleotides are not considered in the calculation of sequence identity when the sequence is modified by “consisting essentially of.”

The specificity of single stranded DNA to hybridize complementary fragments is determined by the stringency of the reaction conditions. Hybridization stringency increases as the propensity to form DNA duplexes decreases. In nucleic acid hybridization reactions, the stringency can be chosen to either favor specific hybridizations (high stringency). Less-specific hybridizations (low stringency) can be used to identify related, but not exact, DNA molecules (homologous, but not identical) or segments.

DNA duplexes are stabilized by: (1) the number of complementary base pairs, (2) the type of base pairs, (3) salt concentration (ionic strength) of the reaction mixture, (4) the temperature of the reaction, and (5) the presence of certain organic solvents, such as formamide, which decreases DNA duplex stability. A common approach is to vary the temperature: higher relative temperatures result in more stringent reaction conditions. Ausubel et al. (1987) provide an excellent explanation of stringency of hybridization reactions (Ausubel, 1987).

To hybridize under “stringent conditions” describes hybridization protocols in which nucleotide sequences at least 60% homologous to each other remain hybridized. “Small interfering RNA” (“siRNA”) (or “short interfering RNAs”) refers to an RNA (or RNA analog) comprising between about 10-50 nucleotides (or nucleotide analogs) that is capable of directing or mediating RNA interference. An effective siRNA can comprise between about 15-30 nucleotides or nucleotide analogs, between about 16-25 nucleotides, between about 18-23 nucleotides, and even about 19-22 nucleotides.

“Nucleotide analog” or “altered nucleotide” or “modified nucleotide” refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function. Examples of positions of the nucleotide which can be derivitized include the 5 position, e.g., 5-(2-amino)propyl uridine, 5-bromo uridine, 5-propyne uridine, 5-propenyl uridine, etc.; the 6 position, e.g, 6-(2-amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs (Herdewijn, 2000).

“RNA analog” refers to an polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. Oligonucleotides can be linked with linkages which result in a lower rate of hydrolysis of the RNA analog as compared to an RNA molecule with phosphodiester linkages. For example, the nucleotides of the analog can comprise methylenediol, ethylene diol, oxymethylthio, oxyethylthio, oxycarbonyloxy, phosphorodiamidate, phosphoroamidate, and/or phosphorothioate linkages. RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate (mediates) RNA interference.

“RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of target genes.

An RNAi agent having a strand which is “sequence sufficiently complementary to a target mRNA sequence to direct target-specific RNA interference (RNAi)” means that the strand has a sequence sufficient to trigger the destruction of the target mRNA by the RNAi machinery or process.

An “isolated” molecule (e.g., “isolated siRNA” or “isolated siRNA precursor”) refers to a molecule that is substantially free of other cellular material, or culture medium when produced by recombinant techniques, or substantially free of chemical precursors or other chemicals when chemically synthesized.

“Transgene” refers to any nucleic acid molecule that is inserted by artifice into a cell, and becomes part of the genome of the organism that develops from the cell. Such a transgene can include a gene that is partly or entirely heterologous (i.e., foreign) to the transgenic organism, or can represent a gene homologous to an endogenous gene of the organism. “Transgene” also means a nucleic acid molecule that includes one or more selected nucleic acid sequences, e.g., DNAs, that encode one or more engineered RNA precursors, to be expressed in a transgenic organism, e.g., plant, that is partly or entirely heterologous, i.e., foreign, to the transgenic plant, or homologous to an endogenous gene of the transgenic plant, but which is designed to be inserted into the plant's genome at a location that differs from that of the natural gene. A transgene includes one or more promoters and any other DNA, such as introns, necessary for expression of the selected nucleic acid sequence, operably linked to the selected sequence, and can include an enhancer sequence.

Comparing a value, level, feature, characteristic, property, etc. to a suitable 6ontrol means comparing that value, level, feature, characteristic, or property to any control or standard familiar to one of ordinary skill in the art useful for comparison purposes. A suitable control can be a value, level, feature, characteristic, property, etc. determined prior to performing an RNAi methodology. For example, a transcription rate, mRNA level, translation rate, protein level, biological activity, cellular characteristic or property, genotype, phenotype, etc. can be determined prior to introducing a RNAi agent of the invention into a cell or organism. A suitable control can be a value, level, feature, characteristic, property, etc. determined in a cell or organism, e.g., a control or normal cell or organism, exhibiting, for example, normal traits. A control can also be a predefined value, level, feature, characteristic, property, etc.

### Practicing the Invention

The invention includes methods of silencing Solanum tuberosum or sweet potato VI genes, wherein a Solanum tuberosum or sweet potato plant is transformed with nucleic acids capable of silencing the VI genes. Silencing the VI genes can be done conveniently by sub-cloning the polynucleotides of SEQ ID NOs: 5, 6, 9-11, and 23-24 into RNAi vectors. The methods described herein can be used to (1) control cold-induced sweeting in potato or sweet potato; and (2) reduce acrylamide levels in processed products from potato or sweet potato.

RNA interference (RNAi) in plants (i.e., post-transcriptional gene silencing (PTGS)) is an example of a broad family of phenomena collectively called RNA silencing (Hannon, 2002). The unifying features of RNA silencing phenomena are the production of small (21-26 nt) RNAs that act as specificity determinants for down-regulating gene expression (Hamilton and Baulcombe 1999; Hammond et al. 2000; Parrish et al. 2000; Zamore et al. 2000; Djikeng et al. 2001; Parrish and Fire 2001; Tijsterman et al. 2002) and the requirement for one or more members of the Argonaute family of proteins (or PPD proteins, named for their characteristic PAZ and Piwi domains) (Tabara et al. 1999; Fagard et al. 2000; Hammond et al. 2001; Hutvagner and Zamore 2002; Kennerdell et al. 2002; Martinez et al. 2002a; Pal-Bhadra et al. 2002; Williams and Rubin 2002). Small RNAs are generated in animals by members of the Dicer family of double-stranded RNA (dsRNA)-specific endonucleases (Bernstein et al. 2001; Billy et al. 2001; Grishok et al. 2001; Ketting et al. 2001). Dicer family members are large, multidomain proteins that contain putative RNA helicase, PAZ, two tandem ribonuclease III (RNase III), and one or two dsRNA-binding domains. The tandem RNase III domains are believed to mediate endonucleolytic cleavage of dsRNA into small interfering RNAs (siRNAs), the mediators of RNAi. In Drosophila and mammals, siRNAs, together with one or more Argonaute proteins, form a protein-RNA complex, the RNA-induced silencing complex (RISC), which mediates the cleavage of target RNAs at sequences with extensive complementarity to the siRNA (Zamore et al., 2000).

In addition to Dicer and Argonaute proteins, RNA-dependent RNA polymerase (RdRP) genes are required for RNA silencing in PTGS initiated by transgenes that overexpress an endogenous mRNA in plants (Zamore et al., 2000), although transgenes designed to generate dsRNA bypass this requirement (Beclin et al., 2002).

Dicer in animals and CARPEL FACTORY (CAF, a Dicer homolog) in plants also generate microRNAs (miRNAs), 20-24-nt, single-stranded non-coding RNAs thought to regulate endogenous mRNA expression (Park et al., 2002). miRNAs are produced by Dicer cleavage of stem-loop precursor RNA transcripts (pre-miRNAs); the miRNA can reside on either the 5′ or 3′ side of the double-stranded stem. Generally, plant miRNAs have far greater complementarity to cellular mRNAs than is the case in animals, and have been proposed to mediate target RNA cleavage via an RNAi-like mechanism (Llave et al., 2002; Rhoades et al., 2002).

In plants, RNAi can be achieved by a transgene that produces hairpin RNA (hpRNA) with a dsRNA region (Waterhouse and Helliwell, 2003). Although antisense-mediated gene silencing is an RNAi-related phenomenon (Di Serio et al., 2001), hpRNA-induced RNAi is more efficient (Chuang and Meyerowitz, 2000). In an hpRNA-producing vector, the target gene is cloned as an inverted repeat spaced with an unrelated sequence as a spacer and is driven by a strong promoter, such as the 35S CaMV promoter for dicots or the maize ubiquitin 1 promoter for monocots. When an intron is used as the spacer, essential for stability of the inverted repeat in Escherichia coli, efficiency becomes high: almost 100% of transgenic plants show gene silencing (Smith et al., 2000; Wesley et al., 2001). RNAi can be used against a vast range of targets; 30 and 50 untranslated regions (UTRs) as short as 100 nt can be efficient targets of RNAi (Kusaba, 2004).

For genome-wide analysis of gene function, a vector for high-throughput cloning of target genes as inverted repeats, which is based on an LR clonase reaction, is useful (Wesley et al., 2001). Another high-throughput RNAi vector, based on “spreading of RNA targeting” (transitive RNAi) from an inverted repeat of a heterologous 30 UTR (Brummell et al., 2003). A chemically regulated RNAi system has also been developed (Guo et al., 2003).

Virus-induced gene silencing (VIGS) is another approach often used to analyse gene function in plants (Waterhouse and Helliwell, 2003). RNA viruses generate dsRNA during their life cycle by the action of virus-encoded RdRP. If the virus genome contains a host plant gene, inoculation of the virus can trigger RNAi against the plant gene. This approach is especially useful for silencing essential genes that would otherwise result in lethal phenotypes when introduced in the germplasm. Amplicon is a technology related to VIGS (Waterhouse and Helliwell, 2003). It uses a set of transgenes comprising virus genes that are necessary for virus replication and a target gene. Like VIGS, amplicon triggers RNAi but it can also overcome the problems of host-specificity of viruses (Kusaba, 2004).

In addition, siRNAs and hpRNAs can be synthesized and then introduced into host cells. The polynucleotides of SEQ ID NOs:5, 6, 9-11 and 23-24 can be prepared by conventional techniques, such as solid-phase synthesis using commercially available equipment, such as that available from Applied Biosystems USA Inc. (Foster City, Calif.; USA), DuPont, (Wilmington, Del.; USA), or Milligen (Bedford, Mass.; USA). Modified polynucleotides, such as phosphorothioates and alkylated derivatives, can also be readily prepared by similar methods known in the art (Ruth, 1990).

### I. RNAi Vectors

Excellent guidance can be found in Preuss and Pikaard regarding RNAi vectors (Preuss and Pikaard, 2004). Several families of RNAi vectors that use Agrobacterium tumefaciens-mediated delivery into plants widely available. All share the same overall design, but differ in terms of selectable markers, cloning strategies and other elements (Table 1). A typical design for an RNAi-inducing transgene comprises a strong promoter (as well-known to those of skill in the art, such as Cauliflower Mosaic Virus 35S promoter) driving expression of sequences matching the targeted mRNA(s). These targeting sequences are cloned in both orientations flanking an intervening spacer, which can be an intron or a spacer sequence that will not be spliced. For stable transformation, a selectable marker gene, such as herbicide resistance or antibiotic resistance, driven by a plant promoter, is included adjacent to the RNAi-inducing transgene. The selectable marker gene plays no role in RNAi but allows transformants to be identified by treating seeds, whole plants or cultured cells with herbicide or antibiotic. For transient expression experiments, no selectable marker gene would be necessary. In constructs for use in A. tumefaciens-mediated delivery, the T-DNA is flanked by a left border (LB) and right border (RB) sequence that delimit the segment of DNA to be transferred. For stable transformation mediated by means other than A. tumefaciens, LB and RB sequences are irrelevant (Preuss and Pikaard, 2004).

TABLE 1
Exemplary vectors for stable transformation for hpRNA production

| | pFGC5941 | pMCG161 | pHannibal | pHELLSGATE |
|---|---|---|---|---|
| Organism | Dicots | Monocots | Dicots | Dicots |
| Cloning | restriction | restriction | restriction | GATEWAY ® |
| Method | digest/ligation | digest/ligation | digest/ligation | recombination |
| | | | | (Invitrogen) |
| Bacterial | kanamycin | chloramphenicol | spectinomycin | spectinomycin |
| Selection | | | | |
| Plant | Basta | Basta | chloramphenicol | Kanamycin |
| Selection | | | | |
| dsRNA | CaMV 35S | CaMV 35S | CaMV 35S | CaMV 35S |
| promoter | | | | |
| Inverted | ChsA intron | Waxy intron | Pdk intron | Pdk intron |
| repeat | | | | |
| spacer | | | | |

Two vectors are especially useful, pHANNIBAL and pHELLSGATE (Helliwell et al., 2005; Wesley et al., 2001). pHELLSGATE vectors are also described in U.S. Pat. No. 6,933,146 and US Patent Publication 2005/0164394. The pHANNIBAL vector has T-DNA (the portion of the plasmid transferred to the plant genome via Agrobacterium-mediated transformation) that includes a selectable marker gene and a strong promoter upstream of a pair of multiple cloning sites flanking an intron. This structure allows cloning sense and antisense copies of target sequence, separated by the intron. A derivative of the pHANNIBAL vector, pHELLSGATE2, facilitates high-throughput cloning of targeting sequences. The efficiency of pHELLSGATE vectors provides a potential advantage for large scale projects seeking to knock down entire categories of genes. In this vector, the pHANNIBAL vector was modified by replacing the polylinkers with aatB site-specific recombination sequence. pHELLSGATE8 is identical to pHELLSGATE2 but contains the more efficient aatP recombination sites.

Another set of RNAi vectors originally designed for Arabidopsis and maize are freely available through the Arabidopsis Biological Resource Center (ABRC, Ohio State University, Columbus, Ohio) and were donated by the Functional Genomics of Plant Chromatin Consortium (Gendler et al., 2008). Vectors pFGC5941 and pMCG161 include within the T-DNA a selectable marker gene, phosphinothricin acetyl transferase, conferring resistance to the herbicide Basta, and a strong promoter (the 35S promoter of Cauliflower Mosaic Virus) driving expression of the RNAi-inducing dsRNA. Introduction of target sequences into the vector requires two cloning steps, making use of polylinkers flanking a Petunia chalcone synthase intron, an overall design similar to pHANNIBAL. Other ChromDB RNAi vectors, such as pGSA1131, pGSA1165, pGSAl204, pGSA1276, and pGSA1252, pGSA1285, offer kanamycin or hygromycin resistance as plant selectable markers, instead of Basta resistance, and a non-intronic spacer sequence instead of the chalcone synthase intron. The ChromDB vectors are based on pCAMBIA plasmids developed by the Center for Application of Molecular Biology to International Agriculture (CAMBIA; Can berra, Australia). These plasmids have two origins of replication, one for replication in Agrobacterium tumefaciens and another for replication in E. coli. Thus, all cloning steps can be conducted in E. coli prior to transformation (Preuss and Pikaard, 2004).

### II. Design of Targeting Sequences (Preuss and Pikaard, 2004)

RNAi vectors are typically designed such that the targeting sequence corresponding to each of the inverted repeats is 300-700 nucleotides in length; however, a stretch of perfect complementarity larger than 14 nucleotides appears absolutely required; 20 nucleotides is a convenient minimum. Success is more easily achieved when the dsRNA targeting sequence is 300-700 nucleotides. Exemplary targeting sequences of the invention include those of SEQ ID NOs: 5, 6, 9-11, and 23-24, and those having at least 90%-99% sequence (e.g., 91%, 92%, 93%, 94%, 95%, 96%, 97%, 98%) identity thereto (Table 2), as well as any 20 contiguous nucleotides of SEQ ID NO:4 (Table 3) or those having at least 90%-99% sequence (e.g., 91%, 92%, 93%, 94%, 95%, 96%, 97%, 98%) identity thereto.

TABLE 2
Exemplary dsRNA targeting sequences

| SEQ ID NO | Sequence | |
|---|---|---|
| 5 | ttatgcgtgg tccaatgcta | |
| 6 | aacccaattc cacaatccaa | |
| 9 | acaggggcta gcgtgactgc ctccgtcaag atttggtcac ttgagtcggc taatattcga | 60 |
| | tccttcccct tgcaagactt gtaattcatc aagccatatc ttcttcattc tttttttcat | 120 |
| | ttgaaggtta tttcaccgat gtcccatcaa gaaagggaag agagggagaa tatgtagtgt | 180 |
| | tatactctac ttattcgcca ttttagtgat ttttctactg gacttttgct attcgccata | 240 |
| | aggtttagtt gttgtctagc aatgtcagca gcggggcgga tctatagtgt aatgtatggg | 300 |
| | ttcctggaaa ccgaataggt cttacttgga ttttatgtaa actaagaaaa ttcagcaaat | 360 |
| | acatacaaat aatttatcga tttcttattg ctggtgagga ttcggttccc tggcagttac | 420 |
| | aaaactaacc atgggcacct aaatacttgg ggcaacgaga ttgacatttg agcttatgca | 480 |
| | gttgcttaga gcacgtgatt tcgccg | 506 |
| 10 | actgggtcaa gtacaaaggc aacccggttc tggttcctcc acccggcatt ggtgtcaagg | 60 |
| | actttagaga cccgaccact gcttggaccg gaccccaaaa tgggcaatgg cttttaacaa | 120 |
| | tcgggtctaa gattggtaaa acgggtattg cacttgttta tgaaacttcc aacttcacaa | 180 |
| | gctttaagct attggatgaa gtgctgcatg cggttccggg tacgggtatg tgggagtgtg | 240 |
| | tggactttta cccggtatcg actgaaaaaa caaacgggtt ggacacatca tataacggcc | 300 |
| | cgggtgtaaa gcatgtgtta aaagcaagtt tagatgacaa taagcaagat cactatgcta | 360 |
| | ttgggacgta tgacttgaca aagaacaaat ggacacccga taacccggaa ttggattgtg | 420 |
| | gaattgggtt gaagctggat tatgggaaat attatgcatc aaagacattt tatgacccga | 480 |
| | agaaacaacg aagag | 495 |
| 11 | gaaagcttaa gaggcggtga tcctattgtt aagcaagtca atcttcaacc aggttcaatt | 60 |
| | gagctactcc atgttgactc agctgcagag ttggatatag aagcctcatt tgaagtggac | 120 |
| | aaagtcgcgc tccagggaat aattgaagca gatcatgtag gtttcagctg ctctactagt | 180 |
| | ggaggtgctg ctagcagagg cattttggga ccatttggtg tcgttgtaat tgctgatcaa | 240 |
| | acgctatctg agctaacgcc agtttacttc tacatttcta aaggagctga tggccgagct | 300 |
| | gagactcact tctgtgctga tcaaaccaga tcctcagagg ctccgggagt tgctaaacaa | 360 |
| | gtttatggta gttcagtacc cgtgttggac ggtgaaaaac attcgatgag attattggtg | 420 |
| | gaccactcaa ttgtggagag ctttgctcaa ggaggaagaa cagtcataac atcgcgaatt | 480 |
| | tacccaacaa aggcagtgaa tggagcag | 508 |
| 23 | gcacgagtat ggccacccag taccattcca gttatgaccc ggaaaactcc gcctcccatt | 60 |
| | acacattcct cccggatcaa cccgattccg gccaccggaa gtcccttaaa atcatctccg | 120 |
| | gcattttcct ctcctctttc cttttgcttt ctgtagcctt ctttccgatc ctcaacaacc | 180 |
| | agtcaccgga cttgcagagt aactcccgtt cgccggcgcc gccgtcaaga ggtgtttctc | 240 |
| | agggagtctc cgataagact tttcgagatg tcgtcaatgc tagtcacgtt tcttatgcgt | 300 |
| | ggtccaatgc tatgcttagc tggcaaagaa ctgcttacca ttttcaacct caaaaaaatt | 360 |
| | ggatgaacga tcctaatggt ccattgtacc acaagggatg gtatcatctt ttttatcaat | 420 |
| | acaatccaga ttcagctatt tggggaaata tcacatgggg ccatgccgta tccaaggact | 480 |
| | tgatccactg gctctacttg ccttttgcca tggttcctga tcaatggtac gatataaacg | 540 |
| | gtgtctggac tgggtccgct accatcctac ccgatggtca gatcatgatg ctttataccg | 600 |
| | gtgacactga tgattatgta caagtgcaaa atcttgcgta ccccaccaac ttatctgatc | 660 |
| | ctctccttct ag | 672 |
| 24 | actgtgggga tggattgggg aaactgatag tgaatctgct gacctgcaga agggatgggc | 60 |
| | atctgtacag agtattccaa ggacagtgct ttacgacaag aagacaggga cacatctact | 120 |
| | tcagtggcca gttgaagaaa | 140 |

Naturally-occurring miRNA precursors (pre-miRNA) have a single strand that forms a duplex stem including two portions that are generally complementary, and a loop, that connects the two portions of the stem. In typical pre-miRNAs, the stem includes one or more bulges, e.g., extra nucleotides that create a single nucleotide “loop” in one portion of the stem, and/or one or more unpaired nucleotides that create a gap in the hybridization of the two portions of the stem to each other.

In hpRNAs, one portion of the duplex stem is a nucleic acid sequence that is complementary to the target mRNA. Thus, engineered RNA precursors include a duplex stem with two portions and a loop connecting the two stem portions. The two stem portions are about 18 or 19 to about 25, 30, 35, 37, 38, 39, or 40 or more nucleotides in length. In plant cells, the stem can be longer than 30 nucleotides. The stem can include much larger sections complementary to the target mRNA (up to, and including the entire mRNA). The two portions of the duplex stem must be sufficiently complementary to hybridize to form the duplex stem. Thus, the two portions can be, but need not be, fully or perfectly complementary. In addition, the two stem portions can be the same length, or one portion can include an overhang of 1, 2, 3, or 4 nucleotides.

hpRNAs of the invention include the sequences of the desired siRNA duplex. The desired siRNA duplex, and thus both of the two stem portions in the engineered RNA precursor, are selected by methods known in the art. These include, but are not limited to, selecting an 18, 19, 20, 21 nucleotide, or longer, sequence from the target gene mRNA sequence from a region 100 to 200 or 300 nucleotides on the 3′ side of the start of translation. In general, the sequence can be selected from any portion of the mRNA from the target gene (such as that of SEQ ID NO:4; Table 3).

TABLE 3
VI polynucleotide sequence (Solanum tuberosum)(SEQ ID NO: 4)

| | |
|---|---|
| gcacgagtat ggccacccag taccattcca gttatgaccc ggaaaactcc gcctcccatt | 60 |
| acacattcct cccggatcaa cccgattccg gccaccggaa gtcccttaaa atcatctccg | 120 |
| gcattttcct ctcctctttc cttttgcttt ctgtagcctt ctttccgatc ctcaacaacc | 180 |
| agtcaccgga cttgcagagt aactcccgtt cgccggcgcc gccgtcaaga ggtgtttctc | 240 |
| agggagtctc cgataagact tttcgagatg tcgtcaatgc tagtcacgtt tcttatgcgt | 300 |
| ggtccaatgc tatgcttagc tggcaaagaa ctgcttacca ttttcaacct caaaaaaatt | 360 |
| ggatgaacga tcctaatggt ccattgtacc acaagggatg gtatcatctt ttttatcaat | 420 |
| acaatccaga ttcagctatt tggggaaata tcacatgggg ccatgccgta tccaaggact | 480 |
| tgatccactg gctctacttg ccttttgcca tggttcctga tcaatggtac gatataaacg | 540 |
| gtgtctggac tgggtccgct accatcctac ccgatggtca gatcatgatg ctttataccg | 600 |
| gtgacactga tgattatgta caagtgcaaa atcttgcgta ccccaccaac ttatctgatc | 660 |
| ctctccttct agactgggtc aagtacaaag gcaacccggt tctggttcct ccacccggca | 720 |
| ttggtgtcaa ggactttaga gacccgacca ctgcttggac cggaccccaa aatgggcaat | 780 |
| ggcttttaac aatcgggtct aagattggta aaacgggtat tgcacttgtt tatgaaactt | 840 |
| ccaacttcac aagctttaag ctattggatg aagtgctgca tgcggttccg ggtacgggta | 900 |
| tgtgggagtg tgtggacttt tacccggtat cgactgaaaa aacaaacggg ttggacacat | 960 |
| catataacgg cccgggtgta aagcatgtgt taaaagcaag tttagatgac aataagcaag | 1020 |
| atcactatgc tattgggacg tatgacttga caaagaacaa atggacaccc gataacccgg | 1080 |
| aattggattg tggaattggg ttgaagctgg attatgggaa atattatgca tcaaagacat | 1140 |
| tttatgaccc gaagaaacaa cgaagagtac tgtggggatg gattggggaa actgatagtg | 1200 |
| aatctgctga cctgcagaag ggatgggcat ctgtacagag tattccaagg acagtgcttt | 1260 |
| acgacaagaa gacagggaca catctacttc agtggccagt tgaagaaatt gaaagcttaa | 1320 |
| gaggcggtga tcctattgtt aagcaagtca atcttcaacc aggttcaatt gagctactcc | 1380 |
| atgttgactc agctgcagag ttggatatag aagcctcatt tgaagtggac aaagtcgcgc | 1440 |
| tccagggaat aattgaagca gatcatgtag gtttcagctg ctctactagt ggaggtgctg | 1500 |
| ctagcagagg cattttggga ccatttggtg tcgttgtaat tgctgatcaa acgctatctg | 1560 |
| agctaacgcc agtttacttc tacatttcta aaggagctga tggccgagct gagactcact | 1620 |
| tctgtgctga tcaaaccaga tcctcagagg ctccgggagt tgctaaacaa gtttatggta | 1680 |
| gttcagtacc cgtgttggac ggtgaaaaac attcgatgag attattggtg gaccactcaa | 1740 |
| ttgtggagag ctttgctcaa ggaggaagaa cagtcataac atcgcgaatt tacccaacaa | 1800 |
| aggcagtgaa tggagcagca cgactcttcg ttttcaacaa tgccacaggg gctagcgtga | 1860 |
| ctgcctccgt caagatttgg tcacttgagt cggctaatat tcgatccttc cccttgcaag | 1920 |
| acttgtaatt catcaagcca tatcttcttc attctttttt tcatttgaag gttatttcac | 1980 |
| cgatgtccca tcaagaaagg gaagagaggg agaatatgta gtgttatact ctacttattc | 2040 |
| gccattttag tgatttttct actggacttt tgctattcgc cataaggttt agttgttgtc | 2100 |
| tagcaatgtc agcagcgggg cggatctata gtgtaatgta tgggttcctg gaaaccgaat | 2160 |
| aggtcttact tggattttat gtaaactaag aaaattcagc aaatacatac aaataattta | 2220 |
| tcgatttctt attgctggtg aggattcggt tccctggcag ttacaaaact aaccatgggc | 2280 |
| acctaaatac ttggggcaac gagattgaca tttgagctta tgcagttgct tagagcacgt | 2340 |
| gatttcgccg g | 2351 |

### III. Methods for Delivering Polynucleotides to Plants and Plant Cells

Suitable methods include any method by which DNA can be introduced into a cell, such as by Agrobacterium or viral infection, direct delivery of DNA such as, for example, by PEG-mediated transformation of protoplasts (Omirulleh et al., 1993), by desiccation/inhibition-mediated DNA uptake, by electroporation, by agitation with silicon carbide fibers, by acceleration of DNA coated particles, etc. In certain embodiments, acceleration methods are preferred and include, for example, microprojectile bombardment.

Technology for introduction of DNA into cells is well-known to those of skill in the art. Four general methods for delivering a gene into cells have been described: (1) chemical methods (Graham and van der Eb, 1973; Zatloukal et al., 1992); (2) physical methods such as microinjection (Capecchi, 1980), electroporation (Fromm et al., 1985; Wong and Neumann, 1982) and the gene gun (Fynan et al., 1993; Johnston and Tang, 1994); (3) viral vectors (Clapp, 1993; Eglitis and Anderson, 1988; Eglitis et al., 1988; Lu et al., 1993); and (4) receptor-mediated mechanisms (Curiel et al., 1991; Curiel et al., 1992; Wagner et al., 1992).

Electroporation can be extremely efficient and can be used both for transient expression of cloned genes and for establishment of cell lines that carry integrated copies of the gene of interest. The introduction of DNA by electroporation is well-known to those of skill in the art. In this method, certain cell wall-degrading enzymes, such as pectin-degrading enzymes, are employed to render the target recipient cells more susceptible to transformation by electroporation than untreated cells. Alternatively, recipient cells are made susceptible to transformation by mechanical wounding. To effect transformation by electroporation one can use either friable tissues such as a suspension culture of cells or embryogenic callus, or alternatively one can transform immature embryos or other organized tissues directly. Cell walls are partially degraded of the chosen cells by exposing them to pectin-degrading enzymes (pectolyases) or mechanically wounded in a controlled manner.

Microprojectile bombardment, a brute force technique, shoots particles coated with the DNA of interest into to plant cells. Exemplary particles include tungsten, gold, and platinum. An advantage of microprojectile bombardment, in addition to it being an effective means of reproducibly obtaining stably transforming monocots, is that protoplast isolation is unnecessary, and a requirement for susceptibility to Agrobacterium infection is not required. For bombardment, cells in suspension are preferably concentrated on filters or solid culture medium. Alternatively, immature embryos or other target cells can be arranged on solid culture medium. The cells are positioned below a macroprojectile stopping plate. If desired, one or more screens are also positioned between the acceleration device and the cells to be bombarded.

Agrobacterium-mediated transfer is a widely applicable system for introducing genes into plant cells because the DNA can be introduced into whole plant tissues, thereby bypassing the need for regeneration of an intact plant from a protoplast. Dafny-Yelin et al. provide an overview of Agrobacterium transformation (Dafny-Yelin and Tzfira, 2007). Agrobacterium plant integrating vectors to introduce DNA into plant cells is well known in the art, such as those described above, as well as others (Rogers et al., 1987). Further, the integration of the Ti-DNA is a relatively precise process resulting in few rearrangements. The region of DNA to be transferred is defined by the border sequences (Jorgensen et al., 1987; Spielmann and Simpson, 1986). Agrobacterium-mediated transformation is most efficient in dicotyledonous plants. A transgenic plant formed using Agrobacterium transformation methods typically contains a single gene on one chromosome. Homozygous transgenic plants can be obtained by sexually mating (selfing) an independent segregant transgenic plant that contains a single added gene, germinating some of the seed produced and analyzing the resulting plants for the targeted trait or insertion.

In some methods, Agrobacterium carrying the gene of interested can be applied to the target plants when the plants are in bloom. The bacteria can be applied via vacuum infiltration protocols in appropriate media, or even simply sprayed onto the blooms.

For RNA-mediated inhibition in a cell line or whole organism, gene expression can be conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentarnycin, hygromycin, kanamycin, lincomycin, methotrexate, phosphinothricin, puromycin, basta, and tetracyclin. Depending on the assay, quantitation of the amount of gene expression allows one to determine a degree of inhibition which is greater than 10%, 33%, 50%, 90%, 95% or 99% as compared to a cell not treated. Lower doses of injected material and longer times after administration of RNAi agent can result in inhibition in a smaller fraction of cells (e.g., at least 10%, 20%, 50%, 75%, 90%, or 95% of targeted cells). Quantitation of gene expression in a cell can show similar amounts of inhibition at the level of accumulation of target mRNA or translation of target protein. As an example, the efficiency of inhibition can be determined by assessing the amount of gene product in the cell; mRNA can be detected with a hybridization probe having a nucleotide sequence outside the region used for the inhibitory double-stranded RNA, or translated polypeptide can be detected with an antibody raised against the polypeptide sequence of that region. Quantitative PCR techniques can also be used.

### Field Evaluation of VI-RNAi Potato Plants

Potato lines having the vacuolar invertase (VI) gene silenced using the RNA-interference (RNAi) methods described herein have been evaluated in fields in Wisconsin, USA. No growth abnormalities have been observed in potato lines produced using the methods of the present invention when compared to control and empty vector lines. Moreover, the RNAi lines produced using the methods of the present invention exhibited no significant differences in yield (p<0.05) compared to control and empty vector lines. Moreover, tubers harvested from the RNAi lines had specific gravity measurements that were consistent (p<0.05) with those of control and empty vector lines. It is well known to those skilled in the art that the specific gravity of tubers (potatoes) is an important determinant of harvest quality. In fact, specific gravity is used in the industry as a reference to judge fry quality, baking characteristics and storability of a tuber (potato).

### Control of Cold-Induced Sweeting in Potato

The methods described herein for silencing the vacuolar invertase (VI) gene using an RNA-interference (RNAi) in order to decrease the level of VI activity in a potato plant compared to its level in a control can be used to control the accumulation or amount of reducing sugars (such as glucose and fructose) in a potato plant during cold storage for any period of time (such as one day, two days, three days, four days, five days, six days, seven days, eight days, nine days, ten days, eleven days, twelve days, thirteen days, fourteen days, fifteen days, sixteen days, seventeen days, eighteen days, nineteen days, twenty days, twenty-one days, etc.).

Methods for controlling the accumulation or amount of reducing sugars during cold storage in a potato comprise the steps of decreasing a level of vacuolar invertase activity in the potato plant relative to a control potato plant using the methods described herein, namely, by introducing to the potato plant an RNAi construct comprising a fragment of at least 20 contiguous nucleotides of a sequence having at least 90% sequence identity to SEQ ID NO:4, and maintaining the plant under conditions sufficient for expression of the RNAi construct thereby decreasing the level of an mRNA that is encoded by a polynucleotide having at least 90% sequence identity to a nucleic acid sequence of SEQ ID NO:4. This method can further comprise assaying the color of a potato product from a potato of the plant after heat processing the potato (such as into a crisp, chip, French fry, potato stick, shoestring potato or other edible potato product). Alternatively, the method can involve assaying the color of the potato product by comparing the product color with the color of a control potato product from a control potato plant. Examples of assays that can be used include visual color rating, such as the one provided herein in Table 6. Chip color can be visually determined using the Potato Chip Color Reference Standards developed by Potato Chip Institute International, Cleveland, Ohio (Douches and Freyer, 1994; Reeves, 1982). A spectrophotometer, such as the Hunterlab Colorflex calorimetric spectrophotometer can also be used to determine the actual color (www.hunterlab.com).

In the above method the RNAi construct comprises a polynucleotide having at least 90% sequence identity to a polynucleotide selected from the group consisting of SEQ ID NOs: 5, 6, 9, 10, 11, 23 and 24. Alternatively, the RNAi construct comprises a polynucleotide having at least 95% sequence identity to a polynucleotide selected from the group consisting of SEQ ID NOs: 5, 6, 9, 10, 11, 23 and 24. Still further alternatively, the RNAi construct comprises a polynucleotide having at least 98% sequence identity to a polynucleotide selected from the group consisting of SEQ ID NOs: 5, 6, 9, 10, 11, 23 and 24.

Still alternatively, the RNAi construct comprises a polynucleotide selected from the group consisting of SEQ ID NOs: 5, 6, 9, 10, 11, 23 and 24.

In this method, the RNAi vector can be introduced into plants using Agrobacterium tumefaciens. The RNAi vector can comprise, for example, a pHELLSGATE vector, such as pHELLSGATE2 or pHELLSGATE8. Plants amenable to the methods of the invention include those from the genus Solanum, such as potato (Solanum tuberosum).

Potatoes harvested from a plant having its level of vacuolar invertase activity decreased pursuant to the methods described herein exhibit a reduction in the accumulation or amount of reducing sugars during cold storage for a period of at least 2 hours when compared to a potato harvested from a control plant in an amount of from about 5% to about 99%, more specifically, from about 5% to about 95%, about 5% to about 90%, about 5% to about 85%, about 5% to about 80%, about 5% to about 75% about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25% or about 5% to about 20%. More specifically, cold storage can be for a period of for a period of at least three hours, at least four hours, at least five hours, at least six hours, at least eight hours, at least ten hours, at least 12 hours, at least 18 hours, at least 24 hours, at least 30 hours, at least 36 hours or longer. Alternatively, potatoes harverted from a plant having its level of vacuolar invertase activity decreased pursuant to the methods described herein exhibit a reduction in the accumulation or amount of reducing sugars during cold storage for a period of at least 2 hours when compared to a potato harvested from a control plant in an amount of about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99%. More specifically, cold storage can be for a period of for a period of at least three hours, at least four hours, at least five hours, at least six hours, at least eight hours, at least ten hours, at least 12 hours, at least 18 hours, at least 24 hours, at least 30 hours, at least 36 hours or longer.

### Reduction of Acrylamide Levels

In another aspect, the invention is directed to a method for controlling acrylamide formation during heat processing of a potato (such as into a crisp, chip, French fry, potato stick, shoestring potato or other edible potato product) from a potato plant. Controlling the acrylamide formation during heat processing of a potato is particularly important when the potato has been subjected to cold storage for any period of time (such as one day, two days, three days, four days, five days, six days, seven days, eight days, nine days, ten days, eleven days, twelve days, thirteen days, fourteen days, fifteen days, sixteen days, seventeen days, eighteen days, nineteen days, twenty days, twenty-one days, etc.).

In this aspect, the method comprises the steps of decreasing a level of vacuolar invertase activity in the potato plant relative to a control potato plant using the methods described herein, namely, by introducing to the potato plant an RNAi construct comprising a fragment of at least 20 contiguous nucleotides of a sequence having at least 90% sequence identity to SEQ ID NO:4, and maintaining the plant under conditions sufficient for expression of the RNAi construct thereby decreasing the level of an mRNA that is encoded by a polynucleotide having at least 90% sequence identity to a nucleic acid sequence of SEQ ID NO:4.

This method can further comprise assaying the level of acrylamide in a heat processed potato product of a potato from a potato plant produced by the above method. It is preferred that the potato being assayed has been subjected to cold storage for a period of at least 2 hours. More specifically, cold storage can be for a period of for a period of at least three hours, at least four hours, at least five hours, at least six hours, at least eight hours, at least ten hours, at least 12 hours, at least 18 hours, at least 24 hours, at least 30 hours, at least 36 hours or longer. For example, chips derived from the potato from a potato plant produced by the above method can be fried in vegetable oil at 183° C./362° F. or 188° C./370° F. or 190° C./190° F. or at 191° C./375° F. for 2 minutes, 30 seconds or 2 minutes or 2 minutes 15 seconds. Fried chips are then allowed to cool down and can be ground into a powder and the powder used for acrylamide analysis. Routine techniques known in the art can be used to determine the acrylamide levels. For example, a combination of mass spectrometry and liquid chromatography can be used to detect acrylamide.

The assaying of the level of acrylamide in the potato product can further comprise comparing the acrylamide level of a potato product derived from a potato from a potato plant produced by the above method and which potato has been subjected to cold storage for a period of at least two hours with an acrylamide level in a control potato product from a control potato plant (namely, a non-RNAi plant). When assayed, potato products derived from a potato from a potato plant produced by the above method will exhibit at least an at least a 5 fold reduction, at least a 6 fold reduction, at least a 7 fold reduction, at least a 8 fold reduction, at least a 9 fold reduction, at least a 10 fold reduction, at least a 11 fold reduction, at least a 12 fold reduction, at least a 13 fold reduction, at least a 14 fold reduction, at least a 15 fold reduction, at least a 20 fold reduction, at least a 25 fold reduction, at least a 30 fold reduction, at least a 35 fold reduction, at least a 40 fold reduction, at least a 45 fold reduction, at least a 50 fold reduction, at least a 55 fold reduction, at least a 60 fold reduction, at least a 65 fold reduction, at least a 70 fold reduction, at least a 75 fold reduction, at least a 80 fold reduction, at least a 85 fold reduction, at least a 90 fold reduction, at least a 95 fold reduction, at least a 100 fold reduction, at least a 150 fold reduction, at least a 200 fold reduction, at least a 250 fold reduction, at least a 300 fold reduction, at least a 350 fold reduction, at least a 400 fold reduction, at least a 450 fold reduction or at least a 500 fold reduction in the level of acrylamide when compared to a potato product from a control potato plant. More specifically, cold storage can be for a period of for a period of at least three hours, at least four hours, at least five hours, at least six hours, at least eight hours, at least ten hours, at least 12 hours, at least 18 hours, at least 24 hours, at least 30 hours, at least 36 hours or longer. Alternatively, the potato products derived from a potato from a potato plant produced by the above method and which potato has been subjected to cold storage for a period of at least two hours when assayed exhibit a 5 to 500 fold reduction, a 5 to 450 fold reduction, a 5 to 400 fold reduction, a 5 to 400 fold reduction, a 5 to 350 fold reduction, a 5 to 300 fold reduction, a 5 to 250 fold reduction, a 5 to 200 fold reduction, a 5 to 150 fold reduction, a 5 to 100 fold reduction, a 5 to 95 fold reduction, a 5 to 90 fold reduction, a 5 to 85 fold reduction, a 5 to 80 fold reduction, a 5 to 75 fold reduction, a 5 to 70 fold reduction, a 5 to 65 fold reduction, a 5 to 60 fold reduction, a 5 to 55 fold reduction, a 5 to 50 fold reduction, a 5 to 45 fold reduction, a 5 to 40 fold reduction, a 5 to 35 fold reduction, a 5 to 30 fold reduction, a 5 to 25 fold reduction, a 5 to 20 fold reduction, a 5 to 15 fold reduction, a 5 to 10 fold reduction, a 10 to 500 fold reduction, a 10 to 450 fold reduction, a 10 to 400 fold reduction, a 10 to 400 fold reduction, a 10 to 350 fold reduction, a 10 to 300 fold reduction, a 10 to 250 fold reduction, a 10 to 200 fold reduction, a 10 to 150 fold reduction, a 10 to 100 fold reduction, a 10 to 95 fold reduction, a 10 to 90 fold reduction, a 10 to 85 fold reduction, a 10 to 80 fold reduction, a 10 to 75 fold reduction, a 10 to 70 fold reduction, a 10 to 65 fold reduction, a 10 to 60 fold reduction, a 10 to 55 fold reduction, a 10 to 50 fold reduction, a 10 to 45 fold reduction, a 10 to 40 fold reduction, a 10 to 35 fold reduction, a 10 to 30 fold reduction, a 10 to 25 fold reduction, a 10 to 20 fold reduction or a 10 to 15 fold reduction in the level of acrylamide when compared to a potato product from a control potato plant. More specifically, cold storage can be for a period of for a period of at least three hours, at least four hours, at least five hours, at least six hours, at least eight hours, at least ten hours, at least 12 hours, at least 18 hours, at least 24 hours, at least 30 hours, at least 36 hours or longer. Still further alternatively, the potato products derived from a potato from a potato plant produced by the above method and which potato has been subjected to cold storage for a period of at least two hours when assayed exhibit levels of acrylamide 25% to 75% less, 25% to 70% less, 25% to 65% less, 25% to 60% less, 25% to 55% less, 25% to 55% less, 25% to 50% less, 25% to 45% less, 25% to 40% less, 25 to 35% less, 30% to 75% less, 30% to 70% less, 30% to 65% less, 30% to 60% less, 30% to 55% less, 30% to 55% less, 30% to 50% less, 30% to 45% less, 25% to 40% less, 30% to 35% less, 35% to 75% less, 35% to 70% less, 35% to 65% less, 35% to 60% less, 35% to 55% less, 35% to 55% less, 35% to 50% less, 35% to 45% less, 35% to 40% less, 40% to 75% less, 40% to 70% less, 40% to 65% less, 40% to 60% less, 40% to 55% less, 40% to 55% less, 40% to 50% less, 40% to 45% less, 45% to 75% less, 45% to 70% less, 45% to 65% less, 45% to 60% less, 45% to 55% less, 45% to 55% less, 45% to 50%, 50% to 75% less, 50% to 70% less, 50% to 65% less, 50% to 60% less or 50% to 55% less, when compared to a potato product from a control potato plant. More specifically, cold storage can be for a period of for a period of at least three hours, at least four hours, at least five hours, at least six hours, at least eight hours, at least ten hours, at least 12 hours, at least 18 hours, at least 24 hours, at least 30 hours, at least 36 hours or longer.

Additionally, it is also believed that when assayed as described above, potato products derived from a potato from a potato plant produced by the above method and which potato has been subjected to cold storage for a period of at least two hours will exhibit levels of acrylamide less than 500 ppb (mg/Kg), less than 400 ppb (mg/Kg), less then 300 ppb (mg/Kg), less then 200 ppb (mg/Kg) or less than less then 100 ppb (mg/Kg). Alternatively, when assayed, the potato products derived from a potato from a potato plant produced by the above method will exhibit levels of acrylamide between about 90 ppb (mg/Kg) to about 500 ppb (mg/Kg), about 100 ppb (mg/Kg) to about 500 ppb (mg/Kg), about 200 ppb (mg/Kg) to about 500 ppb (mg/Kg), about 250 ppb (mg/Kg) to about 500 ppb (mg/Kg), about 100 ppb (mg/Kg) to about 300 ppb (mg/Kg), about 100 ppb (mg/Kg) to about 250 ppb (mg/Kg), about 200 ppb (mg/Kg) to about 300 ppb (mg/Kg), about 250 ppb (mg/Kg) to about 300 ppb (mg/Kg), about 300 ppb (mg/Kg) to about 500 ppb (mg/Kg), or about 400 ppb (mg/Kg) to about 500 ppb (mg/Kg). More specifically, cold storage can be for a period of for a period of at least three hours, at least four hours, at least five hours, at least six hours, at least eight hours, at least ten hours, at least 12 hours, at least 18 hours, at least 24 hours, at least 30 hours, at least 36 hours or longer. Additionally, it is also believed that when assayed as described above, potato products derived from a potato from a potato plant or sweet potato products derived from a sweet potato from a sweet potato plant produced by the above method and which potato or sweet potato has been subjected to or stored at room temperature conditions can exhibit levels of acrylamide less than 1100 ppb (mg/Kg), 1000 ppb (mg/Kg), less than 900 ppb (mg/Kg), less then 800 ppb (mg/Kg), less then 700 ppb (mg/Kg), less than less then 600 ppb (mg/Kg), or less than 500 ppb (mg/Kg). Alternatively, when assayed, the potato products derived from a potato from a potato plant produced by the above method will exhibit levels of acrylamide between about 400 ppb (mg/Kg) to about 1100 ppb (mg/Kg), about 400 ppb (mg/Kg) to about 1000 ppb (mg/Kg), about 400 ppb (mg/Kg) to about 900 ppb (mg/Kg), about 400 ppb (mg/Kg) to about 800 ppb (mg/Kg), about 400 ppb (mg/Kg) to about 700 ppb (mg/Kg), about 500 ppb (mg/Kg) to about 1100 ppb (mg/Kg), about 500 ppb (mg/Kg) to about 1000 ppb (mg/Kg), about 500 ppb (mg/Kg) to about 900 ppb (mg/Kg), about 500 ppb (mg/Kg) to about 800 ppb (mg/Kg) or about 500 ppb (mg/Kg) to about 750 ppb (mg/Kg).

The assaying of the level of acrylamide in the potato product can further comprise comparing the acrylamide level of a potato product derived from a potato from a potato plant produced by the above method and which potato has been stored or subjected to room temperature conditions with an acrylamide level in a control potato product from a control potato plant (namely, a non-RNAi plant). When assayed, potato products derived from a potato from a potato plant produced by the above method will exhibit at least a 1 fold reduction, at least a 2 fold reduction, at least a 3 fold reduction, at least a 4 fold reduction, at least a 5 fold reduction, at least a 6 fold reduction, at least a 7 fold reduction, at least a 8 fold reduction, at least a 9 fold reduction, at least a 10 fold reduction, at least a 11 fold reduction, at least a 12 fold reduction, at least a 13 fold reduction, at least a 14 fold reduction or at least a 15 fold reduction in the level of acrylamide when compared to a potato product from a control potato plant.

Alternatively, the potato products derived from a potato from a potato plant or the sweet potato products derived from a sweet potato from a sweet potato plant produced by the above method and which potato or sweet potato has been stored or subjected to room temperature conditions can exhibit a reduction of at least a 1 to 15 fold reduction, a 2 to 15 fold, a 3 to 15 fold, a 4 to 15 fold, a 5 to 15 fold, a 1 to 14 fold, a 2 to 14 fold, a 3 to 14 fold, a 4 to 14 fold a 5 to 14 fold, a 1 to 13 fold, a 2 to 13 fold, a 3 to 13 fold, a 4 to 13 fold a 5 to 15 fold, a 1 to 12 fold, a 2 to 12 fold, a 3 to 12 fold, a 4 to 12 fold, a 5 to 12 fold, a 1 to 11 fold, a 2 to 11 fold, a 3 to 11 fold, a 4 to 11 fold, a 5 to 11 fold, a 1 to 10 fold, a 2 to 10 fold, a 3 to 10 fold, a 4 to 10 fold or a 5 to 10 fold in the level of acrylamide when compared to a potato product from a control potato plant.

Still further alternatively, the potato products derived from a potato from a potato plant or the sweet potato products derived from a sweet potato from a sweet potato plant produced by the above method and which potato or sweet potato has been stored or subjected to room temperature conditions can levels of acrylamide 25% to 75% less, 25% to 70% less, 25% to 65% less, 25% to 60% less, 25% to 55% less, 25% to 55% less, 25% to 50% less, 25% to 45% less, 25% to 40% less, 25 to 35% less, 30% to 75% less, 30% to 70% less, 30% to 65% less, 30% to 60% less, 30% to 55% less, 30% to 55% less, 30% to 50% less, 30% to 45% less, 25% to 40% less, 30% to 35% less, 35% to 75% less, 35% to 70% less, 35% to 65% less, 35% to 60% less, 35% to 55% less, 35% to 55% less, 35% to 50% less, 35% to 45% less, 35% to 40% less, 40% to 75% less, 40% to 70% less, 40% to 65% less, 40% to 60% less, 40% to 55% less, 40% to 55% less, 40% to 50% less, 40% to 45% less, 45% to 75% less, 45% to 70% less, 45% to 65% less, 45% to 60% less, 45% to 55% less, 45% to 55% less, 45% to 50%, 50% to 75% less, 50% to 70% less, 50% to 65% less, 50% to 60% less or 50% to 55% less, when compared to a potato product from a control potato plant or a sweet potato product from a control sweet potato plant.

The above methods (both the cold storage and room temperature) can further comprise heat processing the potato into a crisp, chip, French fry, potato stick or shoestring potato or other edible potato product.

In the above method the RNAi construct comprises a polynucleotide having at least 90% sequence identity to a polynucleotide selected from the group consisting of SEQ ID NOs: 5, 6, 9, 10, 11, 23 and 24. Alternatively, the RNAi construct comprises a polynucleotide having at least 95% sequence identity to a polynucleotide selected from the group consisting of SEQ ID NOs: 5, 6, 9, 10, 11, 23 and 24. Still further alternatively, the RNAi construct comprises a polynucleotide having at least 98% sequence identity to a polynucleotide selected from the group consisting of SEQ ID NOs: 5, 6, 9, 10, 11, 23 and 24.

Still alternatively, the RNAi construct comprises a polynucleotide selected from the group consisting of SEQ ID NOs: 5, 6, 9, 10, 11, 23 and 24.

In this method, the RNAi vector can be introduced into plants using Agrobacterium tumefaciens. The RNAi vector can comprise, for example, a pHELLSGATE vector, such as pHELLSGATE2 or pHELLSGATE8. Plants amenable to the methods of the invention include those from the genus Solanum, such as potato (Solanum tuberosum).

### Applicability of the Methods Described Herein to Other Crops

The methods described herein are also applicable to other crops such as sweet potato (Ipomoea batatas), yams (family Dioscoreaceae) and Cassaya (Manihot esculenta) as well as foodstuffs derived from sweet potatoes and yams for consumption, such as, but not limited to, crisps, chips (for example, a number of deep fried chips are commercially available among sweet potatoes (such as Blue Mesa Grilled Sweet potato chips, Route II sweet potato chips, National Food Mariquitas Sweet Potato Chips and Zapp's regular sweet potato chips) and Cassaya (such as Tropical Del Campo Iselitas Cassaya chips and Yu-qui-tas cassaya chips), shoestrings (also known as sticks) and fries. Cold-induced sweeting and high levels of acrylamide levels after a period of cold storage is also known to be an issue with respect to sweet potatoes. As used herein, the term “sweet potato product” herein refers to foodstuffs derived from sweet potatoes for consumption, such as, but not limited to, crisps, sweet potato chips, shoestrings (also known as sweet potato sticks) and fries. The above described ranges and values for the reduction of reducing sugars in cold-induced potato and reduction of acrylamide levels described above with respect to potato are also applicable to reduction of said levels in sweet potato, yams and Cassaya. Moreover, all of the assays described above in connection for use with a potato are applicable for use with respect to sweet potatoes.

### Kits

The polynucleotides of SEQ ID NOs:5, 6, 9-11 and 23-24 can be included as part of kits. Such kits comprise one or more of the polynucleotides of the invention. In one embodiment, the polynucleotides of SEQ ID NOs:5, 6, 9-11 and 23-24 are provided in RNAi vectors, and are used to silence VI genes, such as in Solanum tuberosum and other plants, such as sweet potato, yams and Cassaya, having VI genes having at least 90% sequence identity with a polynucleotide sequence selected from the group consisting of SEQ ID NOs:5, 6, 9-11, and 23-24 or other fragment from SEQ ID NO:4.

Kits can also include a control nucleic acids, such as an empty RNAi vector, or a vector with a reporter operably linked to a plant promoter. Kits can also include primers and probes for detecting inserts and mRNA from the transgenes, such as those of SEQ ID NOs:12-22.

Kits can also include amplification reagents, reaction components and/or reaction vessels. One or more of the components of the kit can be lyophilized, and the kit can further include reagents suitable for reconstituting the lyophilized products. The kit can additionally contain instructions for use.

When a kit is supplied, the different components of the composition can be packaged in separate containers and admixed immediately before use. Such packaging of the components separately can permit long-term storage of the active components.

The reagents included in the kits can be supplied in containers of any sort such that the different components are preserved and are not adsorbed or altered by the materials of the container. For example, sealed glass ampoules can contain one of more of the reagents or buffers that have been packaged under a neutral, non-reacting gas, such as nitrogen. Ampoules can consist of any suitable material, such as glass, organic polymers, such as polycarbonate, polystyrene, etc.; ceramic, metal or any other material typically used to hold similar reagents. Other examples of suitable containers include simple bottles that can be fabricated from similar substances as ampoules, and envelopes, that can have foil-lined interiors, such as aluminum or an alloy. Other containers include test tubes, vials, flasks, bottles, syringes, etc.

Kits can also be supplied with instructional materials. Instructions can be printed on paper or other substrate, and/or can be supplied as an electronic-readable medium, such as a floppy disc, CD-ROM, DVD-ROM, Zip disc, videotape, audiotape, etc.

### EXAMPLES

The following examples are for illustrative purposes only and should not be interpreted as limitations of the claimed invention. There are a variety of alternative techniques and procedures available to those of skill in the art which would similarly permit one to successfully perform the intended invention.

### Example 1

### Development of Constructs for Silencing the Potato Vacuolar Acid Invertase Gene

A search for the potato cDNA of the vacuolar acid soluble invertase gene (VI) on the Institute for Genomic Research (TIGR) (now, DFCI—Solanum tuberosum Gene Index) (Quackenbush et al., 2000) and NCBI's GenBank (Benson et al., 1994) resulted in three VI sequences that share 99% nucleotide identity (TC132799; The Gene Index Databases, Dana Farber Cancer Institute, Boston, Mass. 02115; (Quackenbush et al., 2000) (SEQ ID NO:1), L29099 (SEQ ID NO:2) and AY341425 (SEQ ID NO:3); Table 4). Based on these sequences a 2351 by full-length VI cDNA in potato was obtained (Table 4; SEQ ID NO:4). The cDNA sequence extracted from the databases was confirmed by re-sequencing the cDNA sequences amplified from potato cultivar, Katandin, using the following primer sets:

Set 1 (amplifies a 810 by region corresponding to 293-1102 by of SEQ ID NO:4)

F1:

| | | |
|---|---|---|
| ttatgcgtgg tccaatgcta | 20 | (SEQ ID NO: 5) |
| R1: | | |
| aacccaattc cacaatccaa | 20 | (SEQ ID NO: 6) |

Set 2 (amplifies a 866 by region corresponding to 1058-1923 by of SEQ ID NO:4)

F2:

| | | |
|---|---|---|
| caaatggaca cccgataacc | 20 | (SEQ ID NO: 7) |
| R2: | | |
| agtcttgcaa ggggaaggat | 20 | (SEQ ID NO: 8) |

Set 3 (amplifies a 830 by region corresponding to 1438-2267 by of SEQ ID NO:4)

F3:

| | | |
|---|---|---|
| cgctccagggaataattgaa | 20 | (SEQ ID NO: 25) |
| R3: | | |
| tttgtaaactgccagggaacc | 20 | (SEQ ID NO: 26) |

TABLE 4
VI sequences for Solanum tuberosum (SEQ ID NOs: 1-4)
SEQ ID NO: 1 (TC132799)

| | |
|---|---|
| gcacgagtat ggccacccag taccattcca gttatgaccc ggaaaactcc gcctcccatt | 60 |
| acacattcct cccggatcaa cccgattccg gccaccggaa gtcccttaaa atcatctccg | 120 |
| gcattttcct ctcctctttc cttttgcttt ctgtagcctt ctttccgatc ctcaacaacc | 180 |
| agtcaccgga cttgcagagt aactcccgtt cgccggcgcc gccgtcaaga ggtgtttctc | 240 |
| agggagtctc cgataagact tttcgagatg tcgtcaatgc tagtcacgtt tcttatgcgt | 300 |
| ggtccaatgc tatgcttagc tggcaaagaa ctgcttacca ttttcaacct caaaaaaatt | 360 |
| ggatgaacga tcctaatggt ccattgtacc acaagggatg gtatcatctt ttttatcaat | 420 |
| acaatccaga ttcagctatt tggggaaata tcacatgggg ccatgccgta tccaaggact | 480 |
| tgatccactg gctctacttg ccttttgcca tggttcctga tcaatggtac gatataaacg | 540 |
| gtgtctggac tgggtccgct accatcctac ccgatggtca gatcatgatg ctttataccg | 600 |
| gtgacactga tgattatgta caagtgcaaa atcttgcgta ccccaccaac ttatctgatc | 660 |
| ctctccttct agactgggtc aagtacaaag gcaacccggt tctggttcct ccacccggca | 720 |
| ttggtgtcaa ggactttaga gacccgacca ctgcttggac cggaccccaa aatgggcaat | 780 |
| ggcttttaac aatcgggtct aagattggta aaacgggtat tgcacttgtt tatgaaactt | 840 |
| ccaacttcac aagctttaag ctattggatg aagtgctgca tgcggttccg ggtacgggta | 900 |
| tgtgggagtg tgtggacttt tacccggtat cgactgaaaa aacaaacggg ttggacacat | 960 |
| catataacgg cccgggtgta aagcatgtgt taaaagcaag tttagatgac aataagcaag | 1020 |
| atcactatgc tattgggacg tatgacttga caaagaacaa atggacaccc gataacccgg | 1080 |
| aattggattg tggaattggg ttgaagctgg attatgggaa atattatgca tcaaagacat | 1140 |
| tttatgaccc gaagaaacaa cgaagagtac tgtggggatg gattggggaa actgatagtg | 1200 |
| aatctgctga cctgcagaag ggatgggcat ctgtacagag tattccaagg acagtgcttt | 1260 |
| acgacaagaa gacagggaca catctacttc agtggccagt tgaagaaatt gaaagcttaa | 1320 |
| gagcgggtga tcctattgtt aagcaagtca atcttcaacc aggttcaatt gagctactcc | 1380 |
| atgttgactc agctgcagag ttggatatag aagcctcatt tgaagtggac aaagtcgcgc | 1440 |
| tccagggaat aattgaagca gatcatgtag gtttcagctg ctctactagt ggaggtgctg | 1500 |
| ctagcagagg cattttggga ccatttggtg tcgttgtaat tgctgatcaa acgctatctg | 1560 |
| agctaacgcc agtttacttc tacatttcta aaggagctga tggccgagct gagactcact | 1620 |
| tctgtgctga tcaaaccaga tcctcagagg ctccgggagt tgctaaacaa gtttatggta | 1680 |
| gttcagtacc cgtgttggac ggtgaaaaac attcgatgag attattggtg gaccactcaa | 1740 |
| ttgtggagag ctttgctcaa ggaggaagaa cagtcataac atcgcgaatt tacccaacaa | 1800 |
| aggcagtgaa tggagcagca cgactcttcg ttttcaacaa tgccacaggg gctagcgtga | 1860 |
| ctgcctccgt caagatttgg tcacttgagt cggctaatat tcgatccttc cccttgcaag | 1920 |
| acttgtaatt catcaagcca tatcttcttc attctttttt tcatttgaag gttatttcac | 1980 |
| cgatgtccca tcaagaaagg gaagagaggg agaatatgta gtgttatact ctacttattc | 2040 |
| gccattttag tgatttttct actggacttt tgctattcgc cataaggttt agttgttgtc | 2100 |
| tagcaatgtc agcagcgggg cggatctata gtgtaatgta tgggttcctg gaaaccgaat | 2160 |
| aggtcttact tggattttat gtaaactaag aaaattcagc aaatacatac aaataattta | 2220 |
| tcgatttctt attgctggtg aggattcggt tccctggcag ttacaaaact aaccatgggc | 2280 |
| acctaaatac ttggggcaac gagattgaca tttgagctta tgcagttgct tagagcacgt | 2340 |
| gatttcgccg g | 2351 |
| SEQ ID NO: 2 (L29099) | |
| gcacgagtat ggccacgcag taccattcca gttatgaccc ggaaaactcc gcctcccatt | 60 |
| acacattcct cccggatcaa cccgattccg gccaccggaa gtcccttaaa atcatctccg | 120 |
| gcattttcct ctcctctttc cttttgcttt ctgtagcctt ctttccgatc ctcaacaacc | 180 |
| agtcaccgga cttgcagagt aactcccgtt cgccggcgcc gccgtcaaga ggtgtttctc | 240 |
| agggagtctc cgataagact tttcgagatg tcgtcaatgc tagtcacgtt tcttatgcgt | 300 |
| ggtccaatgc tatgcttagc tggcaaagaa ctgcttacca ttttcaacct caaaaaaatt | 360 |
| ggatgaacga tcctaatggt ccattgtacc acaagggatg gtatcatctt ttttatcaat | 420 |
| acaatccaga ttcagctatt tggggaaata tcacatgggg ccatgccgta tccaaggact | 480 |
| tgatccactg gctctacttg ccttttgcca tggttcctga tcaatggtac gatattaacg | 540 |
| gtgtctggac tgggtccgct accatcctac ccgatggtca gatcatgatg ctttataccg | 600 |
| gtgacactga tgattatgta caagtgcaaa atcttgcgta ccccactaac ttatctgatc | 660 |
| ctctccttct agactgggtc aagtacaaag gcaacccggt tctggttcct ccacccggca | 720 |
| ttggtgtcaa ggactttaga gacccgacca ctgcttggac cggaccccaa aatgggcaat | 780 |
| ggcttttaac aatcgggtct aagattggta aaacgggtat tgcacttgtt tatgaaactt | 840 |
| ccaacttcac aagctttaag ctattggatg aagtgctgca tgcggttccg ggtacgggta | 900 |
| tgtgggagtg tgtggacttt tacccggtat cgactgaaaa aacaaacggg ttggacacat | 960 |
| catataacgg cccgggtgta aagcatgtgt taaaagcaag tttagatgac aataagcaag | 1020 |
| atcactatgc tattgggacg tatgacttga caaagaacaa atgcacaccc gataacccgg | 1080 |
| aattggattg tggaattggg ttgaagctgg attatgggaa atattatgca tcaaagacat | 1140 |
| tttatgaccc gaagaaacaa cgaagagtac tgtggggatg gattggggaa actgatagtg | 1200 |
| aatctgctga cctgcagaag ggatgggcat ctgtacagag tattccaagg acagtgcttt | 1260 |
| acgacaagaa gacagggaca catctacttc agtggccagt tgaagaaatt gaaagcttaa | 1320 |
| gaggcggtga tcctattgtt aagcaagtca atcttcaacc aggttcaatt gagctactcc | 1380 |
| atgttgactc agctgcagag ttggatatag aagcctcatt tgaagtggac aaagtcgcgc | 1440 |
| tccagggaat aattgaagca gatcatgtag gtttcagctg ctctactagt ggaggtgctg | 1500 |
| ctagcagagg cattttggga ccatttggtg tcgttgtaat tgctgatcaa aagctatctg | 1560 |
| acgtaacgcc agtttacttc tacatttcta aaggagctga tggtcgagct gagactcact | 1620 |
| tctgtgctga tcaaactaga tcctcagagg ctccgggagt tgctaaacaa gtttatggta | 1680 |
| gttcagtacc cgtgttggac ggtgaaaaac attcgatgag attattggtg gaccactcaa | 1740 |
| ttgtggagag ctttgctcaa ggaggaagaa cagtcataac atcgcgaatt tacccaacaa | 1800 |
| aggcagtgaa tggagcagca cgactcttcg ttttcaacaa tcgcacaggg gctagcgtga | 1860 |
| ctgcctccgt caagatttgg tcacttgagt cggctaatat tcgatccttc cccttgcaag | 1920 |
| acttgtaatt catcaagcca tatcttcttc attctttttt tcatttgaag gttatttcac | 1980 |
| cgatgtccca tcaagaaagg gaagagaggg agaatatgta gtgttatact ctacttattc | 2040 |
| gccattttag tgatttttct actggacttt tgctattcgc cataaggttt agttgttgtc | 2100 |
| tagcaatgtc agcagcgggg cggatctata gtgtaatgta tgggttcctg gaaaccgaat | 2160 |
| aggtcttact tggattttat gtaaactaag aaaattcagc aaatacatac aaataattta | 2220 |
| tcgatttctt attgctggtg aggattcggt tccctggcag ttagaaaact gacatgggca | 2280 |
| cctaaatatt tggggc | 2296 |
| SEQ ID NO: 3 (AY341425) | |
| atggccacgc agtaccattc cagttatgac ccggaaaact ccgcctccca ttacacattc | 60 |
| ctcccggatc aacccgattc cggccaccgg aagtccctta aaatcatctc cggcattttc | 120 |
| ctctcctctt tccttttgct ttctgtagcc ttctttccga tcctcaacaa ccagtcaccg | 180 |
| gacttgcaga gtaactcccg ttcgccggcg ccgccgtcaa gaggtgtttc tcagggagtc | 240 |
| tccgataaga cttttcgaga tgtcgtcaat gctagtcaca tttcttatgc gtggtccaat | 300 |
| gctatgctta gctggcaaag aactgcttac cattttcaac ctcaaaaaaa ttggatgaac | 360 |
| gatcctaatg gtccattgta ccacaaggga tggtatcatc ttttttatca atacaatcca | 420 |
| gattcagcta tttggggaaa tatcacatgg ggccatgccg tatccaagga cttgatccac | 480 |
| tggctctact tgccttttgc catggttcct gatcaatggt acgatataaa cggtgtctgg | 540 |
| actgggtccg ctaccatcct acccgatggt cagatcatga tgctttatac cggtgacact | 600 |
| gatgattatg tacaagtgca aaatcttgcg taccccacca acttatctga tcctctcctt | 660 |
| ctagactggg tcaagtacaa aggcaacccg gttctggttc ctccacccgg cattggtgtc | 720 |
| aaggacttta gagacccgac cactgcttgg accggacccc aaaatgggca atggctttta | 780 |
| acaatcgggt ctaagattgg taaaacgggt attgcacttg tttatgaaac ttccaacttc | 840 |
| acaagcttta agctattggg tgaagtgctg catgcggttc cgggtacggg tatgtgggag | 900 |
| tgtgtggact tttacccggt atcgactgaa aaaacaaacg ggttggacac atcatataac | 960 |
| ggcccgggtg taaagcatgt gttaaaagca agtttagatg acaataagca agatcactat | 1020 |
| gctattggga cgtatgactt gacaaagaac aaatggacac ccgataaccc ggaattggat | 1080 |
| tgtggaattg ggttgaagct ggattatggg aaatattatg catcaaagac attttatgac | 1140 |
| ccgaagaaac aacgaagagt actgtgggga tggattgggg aaactgacag tgaatctgct | 1200 |
| gacctgcaga agggatgggc atctgtacag agtattccaa ggacagtgct ttacgacaag | 1260 |
| aagacaggga cacatctact tcagtggcca gttgaagaaa ttgaaagctt aagagcgggt | 1320 |
| gatcctattg ttaagcaagt caatcttcaa ccaggttcaa ttgagctact ccatgttgac | 1380 |
| tcagctgcag agttggatat agaagcctca tttgaagtgg acaaagtcgc gctccaggga | 1440 |
| ataattgaag cagatcatgt aggtttcagc tgctctacta gtggaggtgc tgctagcaga | 1500 |
| ggcattttgg gaccatttgg tgtcgttgta attgctgatc aaacgctatc tgagctaacg | 1560 |
| ccagtttact tcttcatttc taaaggagct gatggccgag ctgagactca cttctgtgct | 1620 |
| gatcaaacca gatcctcaga ggctccggga gttgctaaac aagtttatgg tagttcagta | 1680 |
| cccgtgttgg acggtgaaaa acattcgatg agattattgg tggaccactc aattgtggag | 1740 |
| agctttgctc aaggaggaag aacagtcata acatcgcgaa tttacccaac aaaggcagtg | 1800 |
| aatggagcag cacgactctt cgttttcaac aatgccacag gggctagcgt gactgcctcc | 1860 |
| gtcaagattt ggtcacttga gtcggctaat attcgatcct tccccttgca agacttgtaa | 1920 |
| SEQ ID NO: 4 (VI, CDNA) | |
| gcacgagtat ggccacccag taccattcca gttatgaccc ggaaaactcc gcctcccatt | 60 |
| acacattcct cccggatcaa cccgattccg gccaccggaa gtcccttaaa atcatctccg | 120 |
| gcattttcct ctcctctttc cttttgcttt ctgtagcctt ctttccgatc ctcaacaacc | 180 |
| agtcaccgga cttgcagagt aactcccgtt cgccggcgcc gccgtcaaga ggtgtttctc | 240 |
| agggagtctc cgataagact tttcgagatg tcgtcaatgc tagtcacgtt tcttatgcgt | 300 |
| ggtccaatgc tatgcttagc tggcaaagaa ctgcttacca ttttcaacct caaaaaaatt | 360 |
| ggatgaacga tcctaatggt ccattgtacc acaagggatg gtatcatctt ttttatcaat | 420 |
| acaatccaga ttcagctatt tggggaaata tcacatgggg ccatgccgta tccaaggact | 480 |
| tgatccactg gctctacttg ccttttgcca tggttcctga tcaatggtac gatataaacg | 540 |
| gtgtctggac tgggtccgct accatcctac ccgatggtca gatcatgatg ctttataccg | 600 |
| gtgacactga tgattatgta caagtgcaaa atcttgcgta ccccaccaac ttatctgatc | 660 |
| ctctccttct agactgggtc aagtacaaag gcaacccggt tctggttcct ccacccggca | 720 |
| ttggtgtcaa ggactttaga gacccgacca ctgcttggac cggaccccaa aatgggcaat | 780 |
| ggcttttaac aatcgggtct aagattggta aaacgggtat tgcacttgtt tatgaaactt | 840 |
| ccaacttcac aagctttaag ctattggata aagtgctgca tgcggttccg ggtacgggta | 900 |
| tgtgggagtg tgtggacttt tacccggtat cgactgaaaa aacaaacggg ttggacacat | 960 |
| catataacgg cccgggtgta aagcatgtgt taaaagcaag tttagatgac aataagcaag | 1020 |
| atcactatgc tattgggacg tatgacttga caaagaacaa atggacaccc gataacccgg | 1080 |
| aattggattg tggaattggg ttgaagctgg attatgggaa atattatgca tcaaagacat | 1140 |
| tttatgaccc gaagaaacaa cgaagagtac tgtggggatg gattggggaa actgatagtg | 1200 |
| aatctgctga cctgcagaag ggatgggcat ctgtacagag tattccaagg acagtgcttt | 1260 |
| acgacaagaa gacagggaca catctacttc agtggccagt tgaagaaatt gaaagcttaa | 1320 |
| gaggcggtga tcctattgtt aagcaagtca atcttcaacc aggttcaatt gagctactcc | 1380 |
| atgttgactc agctgcagag ttggatatag aagcctcatt tgaagtggac aaagtcgcgc | 1440 |
| tccagggaat aattgaagca gatcatgtag gtttcagctg ctctactagt ggaggtgctg | 1500 |
| ctagcagagg cattttggga ccatttggtg tcgttgtaat tgctgatcaa acgctatctg | 1560 |
| agctaacgcc agtttacttc tacatttcta aaggagctga tggccgagct gagactcact | 1620 |
| tctgtgctga tcaaaccaga tcctcagagg ctccgggagt tgctaaacaa gtttatggta | 1680 |
| gttcagtacc cgtgttggac ggtgaaaaac attcgatgag attattggtg gaccactcaa | 1740 |
| ttgtggagag ctttgctcaa ggaggaagaa cagtcataac atcgcgaatt tacccaacaa | 1800 |
| aggcagtgaa tggagcagca cgactcttcg ttttcaacaa tgccacaggg gctagcgtga | 1860 |
| ctgcctccgt caagatttgg tcacttgagt cggctaatat tcgatccttc cccttgcaag | 1920 |
| acttgtaatt catcaagcca tatcttcttc attctttttt tcatttgaag gttatttcac | 1980 |
| cgatgtccca tcaagaaagg gaagagaggg agaatatgta gtgttatact ctacttattc | 2040 |
| gccattttag tgatttttct actggacttt tgctattcgc cataaggttt agttgttgtc | 2100 |
| tagcaatgtc agcagcgggg cggatctata gtgtaatgta tgggttcctg gaaaccgaat | 2160 |
| aggtcttact tggattttat gtaaactaag aaaattcagc aaatacatac aaataattta | 2220 |
| tcgatttctt attgctggtg aggattcggt tccctggcag ttacaaaact aaccatgggc | 2280 |
| acctaaatac ttggggcaac gagattgaca tttgagctta tgcagttgct tagagcacgt | 2340 |
| gatttcgccg g | 2351 |

Three different sequences, 506 by (SEQ ID NO:9, nucleotides 1845-2351 of SEQ ID NO:4, single underline in Table 4), 495 by (SEQ ID NO:10; nucleotides 673-1168 of SEQ ID NO:4, double-underscore in Table 4), and 508 by (SEQ ID NO:11; nucleotides 1310-1818 of SEQ ID NO:4, boldface in Table 4), respectively, were selected for silencing constructs design. All cDNA fragments were amplified from Katandin using Platinum Taq DNA polymerase (Invitrogen, Carlsbad, Calif.) with 35 cycles of heat denaturation at 95° C. for 30 seconds, annealing at 60° C. for 30 seconds and extension at 72° C. for 1 minute after an initial heat denaturation at 95° C. for 40 seconds. SEQ ID NO:9 (a 506-bp cDNA fragment) as amplified using primer set 4 (SEQ ID NOs:12-13):

F4:

| | | |
|---|---|---|
| caccacaggg gctagcgtga ctgc | 24 | (SEQ ID NO: 12) |
| R4: | | |
| cggcgaaatc acgtgctcta ag | 22 | (SEQ ID NO: 13) |

Similarly, SEQ ID NO:10 (a 495 by cDNA fragment) was amplified using primer set 5 (SEQ ID NOs:14-15):

F5:

| | | |
|---|---|---|
| caccactggg tcaagtacaa aggc | 24 | (SEQ ID NO: 14) |
| R5: | | |
| ctcttcgttg tttcttcggg tca | 23 | (SEQ ID NO: 15) |

SEQ ID NO:11 (a 508 by cDNA fragment) was amplified using primer set 6 (SEQ ID NOs:16-17):

F6:

| | | |
|---|---|---|
| caccgaaagc ttaagaggcg gtgatcc | 27 | (SEQ ID NO: 16) |
| R6: | | |
| ctgctccatt cactgccttt gtt | 23 | (SEQ ID NO: 17) |

The amplified PCR products were purified using QIAQUICK® PCR purification kit (Qiagen, Valencia, Calif.), gel verified and cloned into pENTR/D directional TOPO cloning vector (Invitrogen). The directional cloning into pENTR vector was verified by sequencing, and the LR recombination reaction was performed using the pHellsGate8 plasmid (this plasmid is identical to pHellsGate2 as described in (Wesley et al., 2001), except it uses attR sites instead of attP sites). Recombination reaction products were analyzed by restriction digestions (XhoI and XbaI) and sequencing, to ensure that the VI sequences recombined in sense and anti-sense orientations. The Agrobacterium GV3101:pMP90 (Hellens et al., 2000) was transformed with pHellsGate8-VI plasmids by the freeze and thaw method (Sambrook and Russell, 2001), and positive clones were selected on YEP medium containing gentamycin (30 mg/ml) and spectinomycin resistance (50 mg/ml) antibiotics. Transformants of Agrobacterium were confirmed by colony PCR using primer sets 4-6 for each of SEQ ID NOs:9, 10 and 11 independent transformations of the VI gene. Single colonies were selected, grown on liquid YEP medium with appropriate antibiotics (Gen^{R} and Spec^{R}) and used to infect potatoes. Potato stem internode explants from 5-6 week old in-vitro plants of potato variety Katandin were used in potato transformation (Bhaskar et al., 2008; Song et al., 2003; Zeigelhoffer et al., 1999). Kanamycin antibiotic was used as a transgenic plant selection marker.

### Example 2

### Confirmation of Transgenic Plants

Transgenic Katandin lines obtained from the three constructs were first screened for the presence of the Kanamycin resistance selection marker. PCR was performed on genomic DNA isolated from the transgenic lines along with non-transformed controls, using the Kanamycin marker-specific primers (primer set 7; SEQ ID NOs:18 and 19):

F7:

| | | |
|---|---|---|
| ccaacgctat gtcctgatag | 20 | (SEQ ID NO: 18) |
| R7: | | |
| tttgtcaaga ccgacctgtc | 20 | (SEQ ID NO: 19) |

Presence or absence of a single 531 by of PCR product was confirmed in a transgenic plant. PCR was performed for 40 cycles of heat denaturation at 95° C. for 20 seconds, annealing at 53° C. for 30 seconds and extension at 72° C. for 1 minute after an initial heat denaturation at 95° C. for 1 minute. The PCR reaction mix (25 μl) consisted of 1×PCR buffer, 0.1 mM dNTPs, 0.2 μM primers, 1.5 mM MgCl₂, 1 U of Platinum Taq polymerase (Invitrogen) and 1.5 ng of genomic DNA.

### Example 3

### Confirmation of VI Gene Silencing

All transgenic Katandin plants obtained from three independent transformations were screened for silencing of the VI gene by Northern blot hybridizations. Total RNA was isolated from potato leaves using the QIAQUICK® RNA Isolation kit (Qiagen). Approximately 15 μg of RNA was loaded in each lane and resolved on denaturing 1% agarose gel and then transferred to HYBOND™+nylon membrane (Amersham Biosciences, Piscataway, N.J.). SEQ ID NO:20 of VI cDNA sequence (Table 5) was PCR amplified with primer set 8 (SEQ ID NOs:21 and 22):

| F8: | | |
|---|---|---|
| acaggggcta gcgtgactgc | 20 | (SEQ ID NO: 21) |
| R8: | | |
| cggcgaaatc acgtgctcta ag | 22 | (SEQ ID NO: 22) |

The probe was radioactively labeled with 3000 Ci/mmol [³²P] dATP (Amersham) using the STRIP-EZ® DNA kit (Ambion, Austin, Tex.) following manufactuer's instructions. The gel blot membrane was prewashed in 65° C. Church buffer (7% SDS, 0.5M Na₂HPO₄, 1 mM EDTA, pH 7.2) for a minimum of 1 hour. The radioactive probes were denatured and then hybridized to the membrane overnight at 65° C. After the hybridization, membranes were washed twice in 2×SSC and 0.1% SDS for 15 min, twice in 0.2×SSC and 0.1% SDS for 15 min. Signals were detected using a phosphor imager and/or exposed to X-ray film and developed.

TABLE 5
Probe sequence (SEQ ID NO: 20)

| | |
|---|---|
| acaggggcta gcgtgactgc ctccgtcaag atttggtcac ttgagtcggc taatattcga | 60 |
| tccttcccct tgcaagactt gtaattcatc aagccatatc ttcttcattc tttttttcat | 120 |
| ttgaaggtta tttcaccgat gtcccatcaa gaaagggaag agagggagaa tatgtagtgt | 180 |
| tatactctac ttattcgcca ttttagtgat ttttctactg gacttttgct attcgccata | 240 |
| aggtttagtt gttgtctagc aatgtcagca gcggggcgga tctatagtgt aatgtatggg | 300 |
| ttcctggaaa ccgaataggt cttacttgga ttttatgtaa actaagaaaa ttcagcaaat | 360 |
| acatacaaat aatttatcga tttcttattg ctggtgagga ttcggttccc tggcagttac | 420 |
| aaaactaacc atgggcacct aaatacttgg ggcaacgaga ttgacatttg agcttatgca | 480 |
| gttgcttaga gcacgtgatt tcgccg | 506 |

### Example 4

### Potato Tubers Storage and Chip Production

Two replications of 70 independent VI-RNAi Katandin lines along with controls were moved from tissue culture to green house pots. The plants were grown in two separate growth chambers; each contained all the plants from one replication. Growth conditions consisted of 70% humidity, 16-h day/8-h night regime, 19° C./15° C., 500 μmol m⁻² s⁻¹ light was applied. Tubers were harvested from the plants after they had developed full senescence. Fresh tuber weight was measured using all the tubers harvested. All tubers were allowed to remain in dark at room temperature for a week. From each line, 3-6 tubers were chosen and stored in humidity-controlled chambers at 4° C. for up to 180 days (6 months), and the remaining tubers are stored in a dark at room temperature (20° C.). For making potato chips, samples were taken by cutting slices (from apical to basal end of the tuber, 0.65 cm diameter, 1.5 mm thick) from tubers stored both at 20° C. and 4° C. The remaining tuber samples were directly frozen in liquid nitrogen for later determination of invertase enzyme activities and sugar profile. Tuber slices were fried for 2 minutes at 191° C. for observations on the chip color. Chip color was visually determined on a 10-chip sample from each plant line with the use of the Potato Chip Color Reference Standards developed by Potato Chip Institute International, Cleveland, Ohio (Douches and Freyer, 1994; Reeves, 1982).

### Example 5

### Acrylamide Analysis

Chipping experiments were performed on tubers stored at 4° C. for 180 days (6 months) with no reconditioning process involved. Potato tubers were cut lengthwise to obtain slices and fried in vegetable oil at 184° C./362° F. or at 191° C./375° F. for 2 minutes, 30 seconds. Fried chips are allowed to cool down and thoroughly grinded and the powder was used for acrylamide analysis. Samples were submitted to Covance Inc., Madison, Wis. and to the laboratory of Mike Pariza at the University of Wisconsin, Madison, Wis. At Covance, Inc., a combination of Mass Spectrometry and Liquid chromatography was used to detect acrylamide, according to the method developed by the United States Food and Drug Administration (http://www.cfsan.fda.govhdms/acrylami.html), adopted from earlier method (Schuster, 1988). At Mike Pariza's lab, samples were analyzed by modified EPA method described before (Park et al. 2005). The t-test was used to study whether the means of two groups were statistically significant from each other.

### Example 6

### Results—Characterization of VI-RNAi Lines

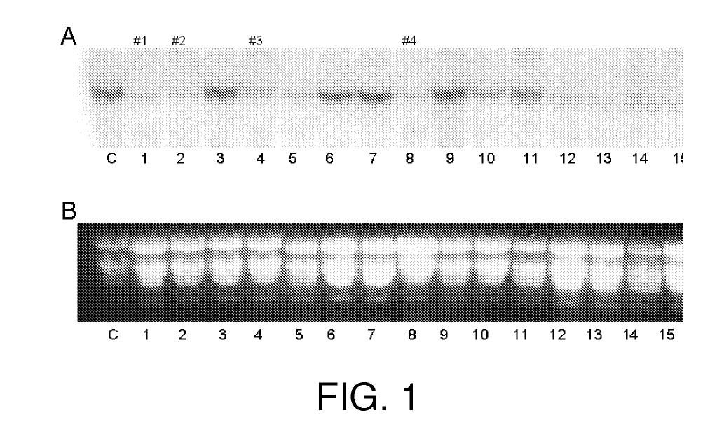

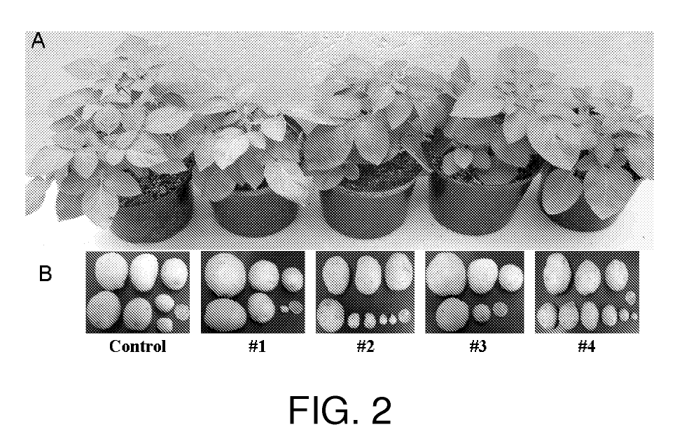

A total of 110 healthy transgenic lines generated from three independent transformations with three different constructs were chosen for analysis (63, 40 and 7 plants resulted from construct of #2, #1, and #3 (SEQ ID NOs: 11, 10 and 9) respectively). Northern blot analysis of transcription of the VI gene was performed on these lines in parallel with non-transformed plants and plants transformed with empty vector (FIG. 1). A 95-99% loss of VI transcript was detected in 23 lines, a 10-90% reduction of the VI gene transcription was detected in 49 lines. The VI gene transcription in the remaining 38 transgenic lines showed no significant difference compared to controls. However, 6 transgenic lines showed almost no detectable transcripts (˜99%) after long exposure of 4 weeks using intensifying screens. A total of 70 representative RNAi lines were chosen for further analysis (6, 12, 45 and 7 lines representing ˜99%, 95-99%, 10-90% silencing and no silencing respectively, respectively). The presence of kanamycin resistance selective marker gene was confirmed by PCR analysis. Two in vitro copies of each of the 70 representative RNAi lines mentioned above were planted, one each in two different greenhouses. Precisely identical growth conditions were maintained in both of the greenhouse rooms from planting until the tubers were harvested. The inventors observed neither distinguishable morphological features nor any tuber phenotypes after harvesting, associated with RNAi silencing of VI gene in these transgenic lines compared with controls (FIG. 2). A few transgenic lines (6 in total) showed stunted phenotypes with aberrant leaf and flower structures. However, these phenotypes did not show any correlation to VI transcript levels and hence attributed to have occurred due to somaclonal variations resulted during in-vitro propagation. Tubers were harvested from the plants after they had developed full senescence. No tuber phenotypes were noticed among VI-RNAi lines compared with controls. Fresh tuber weight was measured using all the tubers harvested. No significant differences were noticed among fresh tuber weights and total tuber number per plant among the transgenic lines compared to controls.

### Example 7

### Results—Chipping Experimental Results

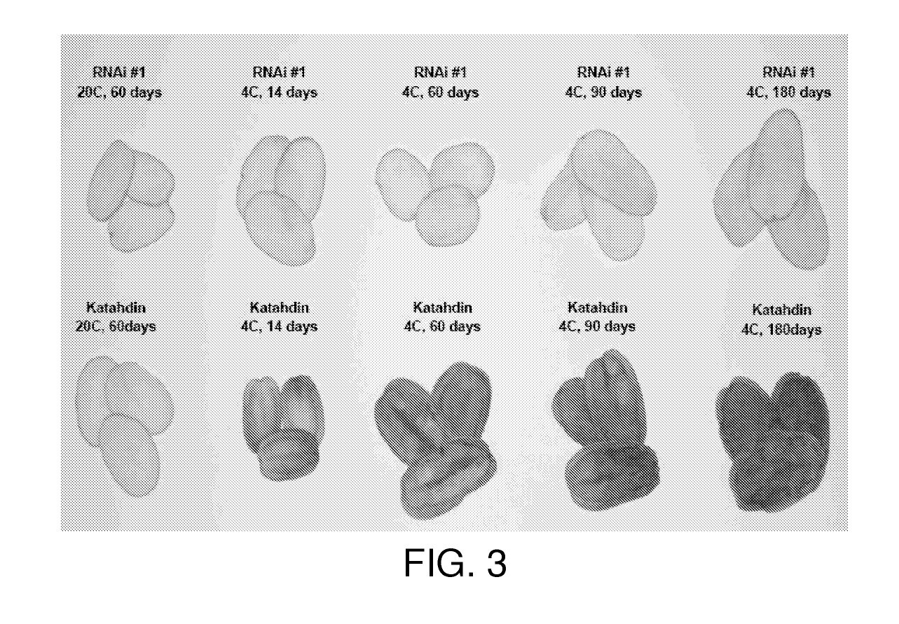

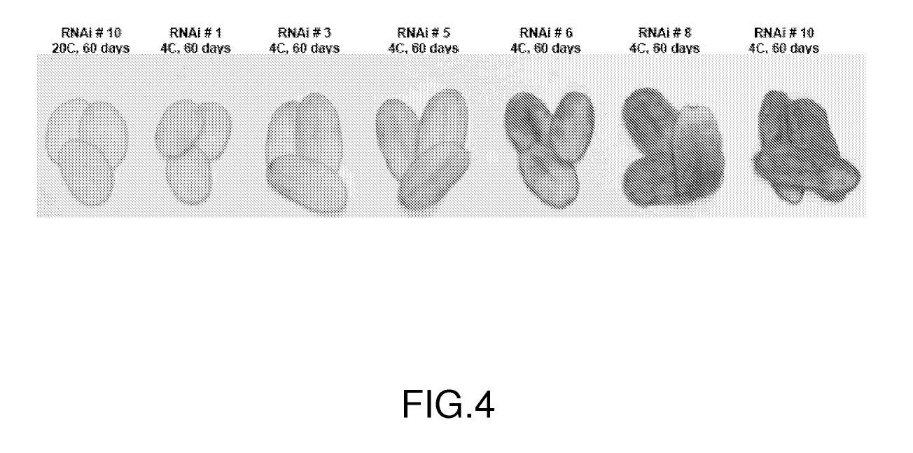

Chipping experiments were performed on tubers stored at 20° C. and tubers taken direct from cold storage (namely, 4° C.). All the chipping experiments were performed on tubers with no reconditioning process involved. Chipping performance of tubers stored at 20° C. was not different between the RNAi lines and the controls. Consistent chip scores of 3.0 were obtained for all lines, on a chip scale from 1 (light) to 10 (dark). Chip scores of 6.0-7.0 and 7.0-8.0 were observed for control tubers at 14 days and 60 days processed directly from 4° C. storage (FIG. 3). Strikingly, chip scores of 3.0 were obtained for tubers from the best RNAi lines processed directly from cold storage at both 14 and 60 days. The light color of the chips correlated with the amount of the VI transcript in the RNAi lines (Table 6, FIG. 4). Chips prepared from ˜99% VI silenced lines (RNAi #1, 2 & 4) consistently gave lighter scores. Interestingly, few of the lines we analyzed (RNAi #3) with ˜90% VI silencing, produced light to medium color chips with scores ranging from 4.0-6.0. However, chipping performance of RNAi lines (#7, 8) was poor even though, these lines showed 50% and 20% VI transcript reduction respectively. Based on this result, the inventors conclude that the levels of VI transcript in the RNAi lines control the amount of reducing sugars in tubers, which determine the color of the potato chips. Chipping performance of RNAi line #7 almost resembled those of the control lines, where no reduction of VI transcript was detected in this line. These results demonstrate that the complete silencing of the VI gene in potato plants can control the accumulation of reducing sugars in cold storage and produce light color chips that conform to current industry standards.

TABLE 6

| | Silencing | Chip scores | | | | | | | | |
|---|---|---|---|---|---|---|---|---|---|---|
| Line | (%) | 20° C./14 d | 20° C./60 d | 4° C./14 d | 4° C./60 d | Performance* | 20° C./90 d | 4° C./90 d | 4° C./180 d | Performance* |
| RNAi #1 | 99 | 3.0 | 3.0 | 3.0 | 3.0 | good | 3.5 | 3.0 | 3.0 | good |
| RNAi #2 | 99 | 3.0 | 3.0 | 3.0 | 3.0 | good | 3.0 | 3.5 | 4.0 | good |
| RNAi #3 | 90 | 3.0 | 3.0 | 5.0 | 5.5 | medium | 3.0 | 5.0 | 5.5 | medium |
| RNAi #4 | 99 | 3.0 | 3.0 | 3.5 | 4.0 | good | 3.0 | 4.0 | 4.0 | good |
| RNAi #5 | 80 | 3.0 | 3.0 | 6.0 | 5.0 | medium | 3.0 | 8.0 | 8.0 | poor |
| RNAi #6 | 60 | 3.0 | 3.0 | 6.0 | 7.0 | poor | 3.0 | 8.0 | 8.0 | poor |
| RNAi #7 | 50 | 3.0 | 3.0 | 6.0 | 7.0 | poor | 3.0 | 8.0 | 8.0 | poor |
| RNAi #8 | 20 | 3.0 | 3.0 | 6.5 | 7.0 | poor | 3.5 | 8.0 | 8.0 | poor |
| RNAi #9 | >10 | 3.0 | 3.0 | 6.0 | 8.0 | poor | 3.0 | 8.0 | 8.0 | poor |
| RNAi #10 | 0 | 3.0 | 3.0 | 6.0 | 8.0 | poor | 3.0 | 8.0 | 8.0 | poor |
| Katahdin | 0 | 3.0 | 3.0 | 6.5 | 8.0 | poor | 4.0 | 8.0 | 8.0 | poor |

*Chipping performance was separated into good (chip score ≦4.5), medium (5-6) and poor (≧7.5)
** Chips were given a half score if their color was indistinguishable between 2 of the 10 color indicators.

Further chipping experiments on tubers taken direct from 4° C. storage at 3 months (90 days) and at 6 months (180 days), produced chip scores of 3.0 to 4.0 for tubers from the best VI RNAi lines. Chips prepared from ˜99% VI silenced lines (RNAi #1, 2 and 4) still produced lighter scores consistently. However, RNAi #3, with ˜90% VI silencing, produced medium color chips with scores ranging from 5.0 to 6.0. As expected from previous 14 and 60 day chipping results, chipping performance of RNAi lines #7, 8 and 9 was poor and produced scores of 8.0. Interestingly, chipping performance of RNAi line #5 was medium at 14 and 60-day chipping experiments but produced poor chipping scores at 90 and 180-day analysis (Table 6). Based on these results the inventors conclude that the levels of VI transcript in the RNAi lines control the amount of reducing sugars in the tubers, which determine the color of the potato chips.

These results support the inventors previous conclusions that the levels of VI transcript in the RNAi lines control the amount of reducing sugars in the tubers, which determine the color of the potato chips. The results also indicate that complete silencing of the VI gene in potato plants can control cold-induced sweetening problem and produce light color chips which are acceptable as per current industry standards.

### Example 8

### Low Acrylamide Levels Among VI RNAi Lines Compared to Controls

In potato, acrylamide is primarily formed by a Maillard-type of reaction among amino acids (Asparagine) and reducing sugars at high frying temperatures (Mottram et al. 2002). Since reducing sugars (glucose and fructose) are among the two major limiting factors during acrylamide formation in potato processed products, we hypothesized that VI silenced RNAi lines would accumulate very low levels of acrylamide compared to controls. The inventors chose cold-stored tubers (4° C. for 14 days and 180 days—no reconditioning) from three VI silenced RNAi lines (RNAi #1, 2, 3) and one Katandin control line (Table 8) for comparing acrylamide levels using the methods described in Example 5. Fried chips were allowed to cool down, thoroughly ground and the resulting powder used for acrylamide analysis. Samples were submitted to Covance Inc., Madison, Wis. and also to the laboratory of Mike Pariza Lab at University of Wisconsin-Madison. At Covance Inc., a combination of mass spectrometry and liquid chromatography was used to detect acrylamide, according to the method developed by the United States Food and Drug Administration (http://www.cfsan.fda.govhdms/acrylami.html), adopted from an earlier method as described in Schuster, 1988. At the Pariza laboratory, University of Wisconsin-Madison, samples were analyzed by modified EPA method as described previously in Park et al. 2005. A t-test was used to study whether the means of two groups were statistically significant from each other.

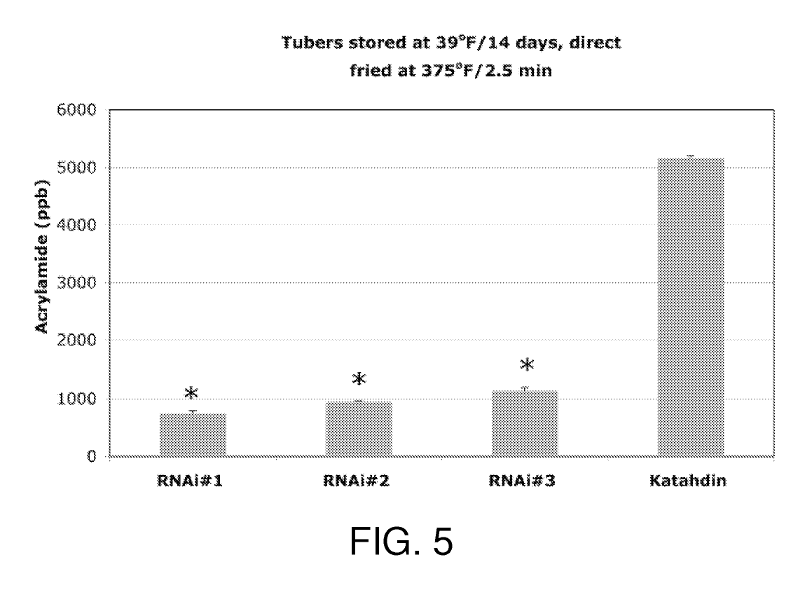

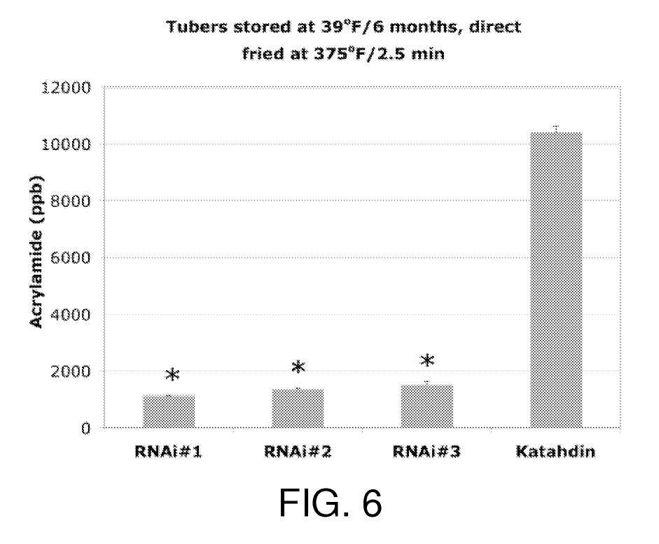

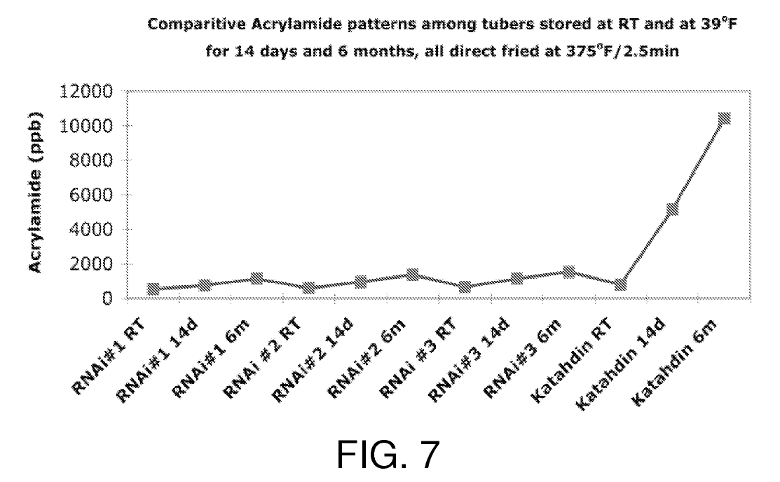

Remarkably, acrylamide levels were significantly reduced among the potato chip samples obtained from VI silenced RNAi lines compared to controls. Due to limited availability of tuber samples, acrylamide levels were measured at higher frying times (2 minutes, 30 seconds) and temperatures (375° F.). Chips fried from RNAi lines showed a 9 to 10-fold reduction of acrylamide levels compared to controls (FIGS. 5 and 6). In particular, acrylamide level in RNAi #1 was as low as 750 ppb compared to non-transformed Katandin control (5160 ppb) at 14 days cold storage (4° C.). The similar line (RNAi #1) produced reduced acrylamide levels of 1130 ppb after 6 month cold storage compared to the control line (10420 ppb). The inventors observed a similar 8 to 12-fold reduction of acrylamide levels among other VI silenced lines (#2 and 3) compared to controls (FIGS. 5 and 6). Interestingly, no change in acrylamide levels was noticed for RNAi line #1 among RT and 14-day cold stored, compared to elevated acrylamide levels among controls during these time points (FIG. 7).

This study demonstrated that VI-RNAi lines that were cold stored at 39° F. for several months could still yield potato chips with relatively low levels of acrylamide (FIG. 7) compared to the controls. There has been no change in acrylamide level after 14-day cold storage in at least one RNAi line (#1) compared to non-transformed control and only a slight increase by 0.5 fold after a prolonged 6-month cold storage at 39° F. This finding indicates that dramatic reductions in VI transcript levels are sufficient enough to guarantee a low-acrylamide product. Currently, no commercial cultivars produce acceptable chips removed from 39° F. storage because excess-reducing sugars will form dark color and acrylamide in Maillard reaction.

### Example 9

### Field Evaluations of VI-RNAi Potato Lines

A total of 60 transgenic lines generated from two independent transformations with two different constructs were chosen for analysis (namely, 20 plants (RNAi #1) generated from SEQ ID NO:11, 20 plants (RNAi #2) generated from SEQ ID NO:11 and 20 plants (RNAi #3) generated from SEQ ID NO: #10, respectively). Control plants included a total of 20 transgenics resulted from empty vector construct (Agrobacterium GV3101:pHellsGate8) and 20 untransformed Katandin plants. 60 other control plants of the varieties Snowden, Russett Burbank, Megachip and Red Norland were also included in field analysis.

All the above-mentioned potato lines were transplanted at both field locations during first and second weeks of June 2009 at Hancock, Wis. (Jun. 2, 2009) and Rhinelander, Wis. (Jun. 9, 2009) locations respectively. At the Hancock, Wis. location, 10 plants of RNAi #1, 10 plants of RNAi #2 and 10 plants of RNAi #3, 10 plants of empty vector, 10 plants of non-transformed controls and 80 control plants of other potato varieties were planted. Similarly at the Rhinelander Wis. location, 10 plants of RNAi #1, 10 plants of RNAi #2 and 10 plants of RNAi #3, 10 plants of empty vector, 10 plants of non-transformed controls and 100 control plants of other potato varieties were planted.

All the plants were manually transplanted in both the field locations with 3 feet space between the rows and 2 feet space within the row between the plants. A Completely Randomized Design (CRD) was followed at both the field locations. The total acerage planted at the Hancock, Wis. location included 30×51 feet (approximately 0.14 acre) and at the Rhinelander, Wis. location it included 22×63 feet (approximately 0.20 acre). Once all the plant materials were transplanted, routine cultivation and management practices followed at both the field locations as described below.

The routine cultivation and management practices followed at the Hancock, Wis. and Rhinelander, Wis. locations were the following:

Fertilizer: April 1, N—P—K—S—Ca in the form of 0-0-0-17S-21Ca (Calcium Sulfate) 70 lb/0.14 acre and N—P—K in the form of 0-0-60 (Potash) 52.5 lb/0.14 acre
Fertilizer: June 16, N—P—K—S in the form of 21-0-0-24S (Ammonium Sulfate) 491b/0.14 acre
Fungicide: June 18, EQUUS ZN fungicide 0.21 pints/0.14 acre
Fungicide: June 26, BRAVO ZN fungicide 0.21 pints/0.14 acre
Fungicide: July 2, EQUUS ZN fungicide 0.21 pints/0.14 acre, Headline EC fungicide 0.84 fluid oz/0.14 acre
Fertilizer: July 9, N—P—K in the form of 46-0-0 (Urea) 31.5 lb/0.14 acre
Fungicide: July 10, ECHO Zn fungicide, 0.21 pints/0.14 acre
Fungicide: July 17, BRAVO Zn 0.21 pints/0.14 acre, ENDURA fungicide 0.35 dry oz/0.14 acre
Fungicide: July 23, ECHO Zn 0.21 pints/0.14 acre
Fungicide: July 30, ECHO Zn 0.21 pints/0.14 acre and Headline fungicide 0.84 fluid oz/0.14 acre
Insecticide: July 31, Coragen 0.7 fluid oz/0.14 acre
Fungicide: August 7, ECHO Zn 0.42 pints/0.14 acre, Tanos fungicide 1.12 dry oz/0.14 acre
Insecticide: August 11, Coragen 0.49 fluid oz/0.14 acre
Fungicide: August 13, ECHO Zn 0.42 pints/0.14 acre, Manzate Pro Stick fungicide 0.03 lb/0.14 acre
Fungicide: August 20, Echo Zn 0.3 pints, Tanos fungicide 1.12 dry oz/0.14 acre
Fungicide: August 27, Manzate Pro Stick fungicide Echo Zn/0.14 acre Sep. 23, 2009-harvest

Irrigation schedule and Rate: Apr. 20, 2009 (Rate in inches: 0.25 inches), May 4, 2009 (0.5), May 18, 2009 (0.5), May 22, 2009 (0.5), Jun. 1, 2009 (0.3), Jun. 2, 2009 (0.25), Jun. 5, 2009 (0.5), Jun. 11, 2009 (0.25), Jun. 15, 2009 (0.5), Jun. 19, 2009 (0.5), Jun. 21, 2009 (0.5), Jun. 23, 2009 (0.5), Jun. 25, 2009 (0.5), Jun. 27, 2009 (0.5). Irrigation schedule continued on every 3^{rd} day @ of 0.5 inches until harvest date on Sep. 23, 2009.

### Field Evaluations of VI-RNAi Potato Lines

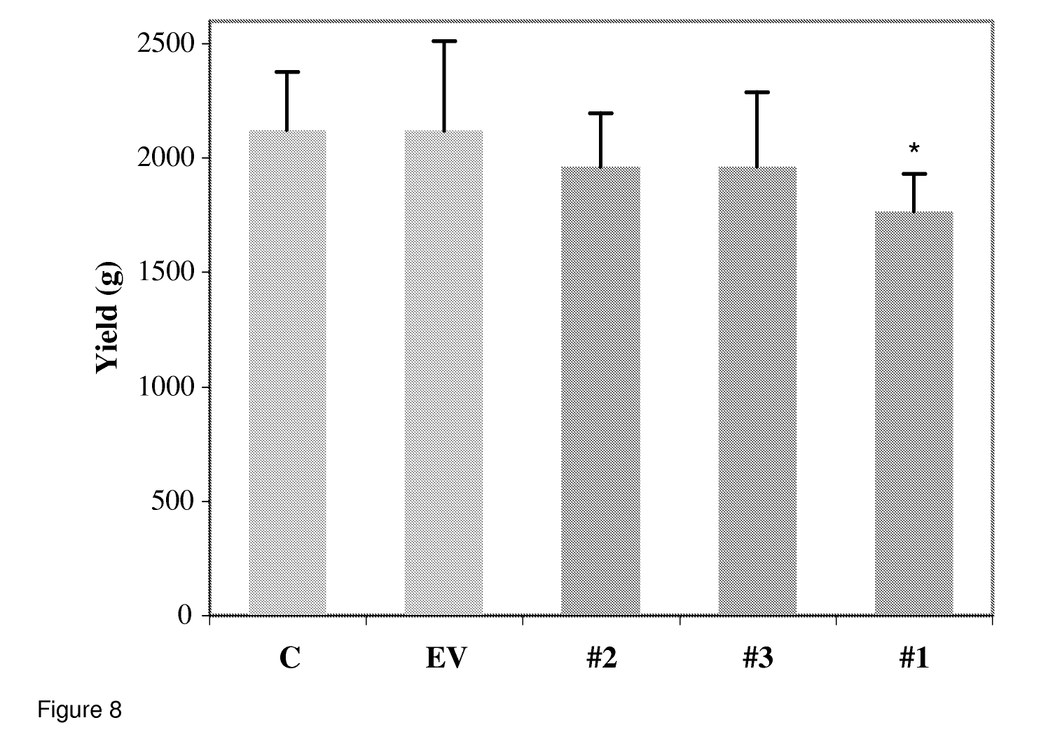

Field evaluations of the VI-RNAi lines were conducted in Wisconsin during summer of 2009 at the Hancock and Rhinelander plant locations. No growth abnormalities were noticed among transgenic VI-RNAi plants compared to control plants. RNAi lines showed no significant yield differences (p<0.05) compared to control and empty vector lines (See, FIG. 8). Specific gravity measurements were performed among field grown transgenic and control tubers. Specific gravity of potatoes is an important determinant of harvest quality. In practice, potato industry uses specific gravity as a reference to judge fry quality, baking characteristics and storability. Specific gravity measurements were determined as follows. Tuber sample size in the range of 10 to 15 lbs (4.5-6.8 kg) was used as an adequate sample size for specific gravity measurements. Selected sample units are first weighed in air and then the same unit is re-weighed suspended in water. Specific gravity was calculated using the following formula:

Specific gravity=Weight in air/(Weight in air−Weight in water).

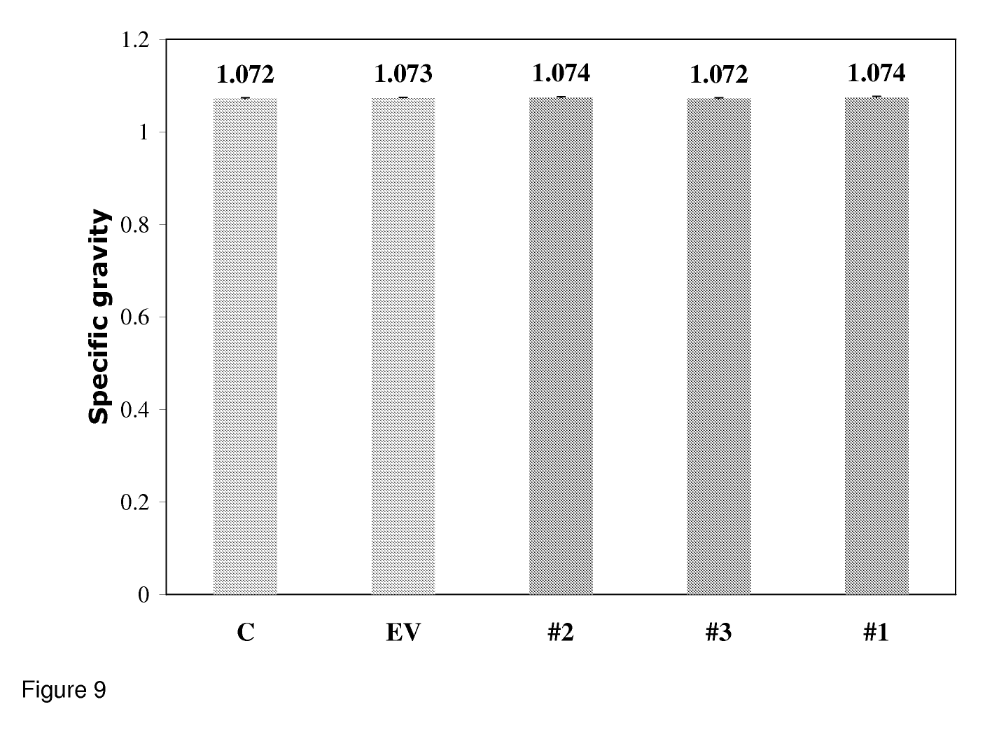

The specific gravity measurements performed as described above were consistent (p<0.05) among transgenic VI-RNAi tubers compared with the controls (See, FIG. 9).

Chipping Performance of Tubers from VI-RNAi Potato Lines

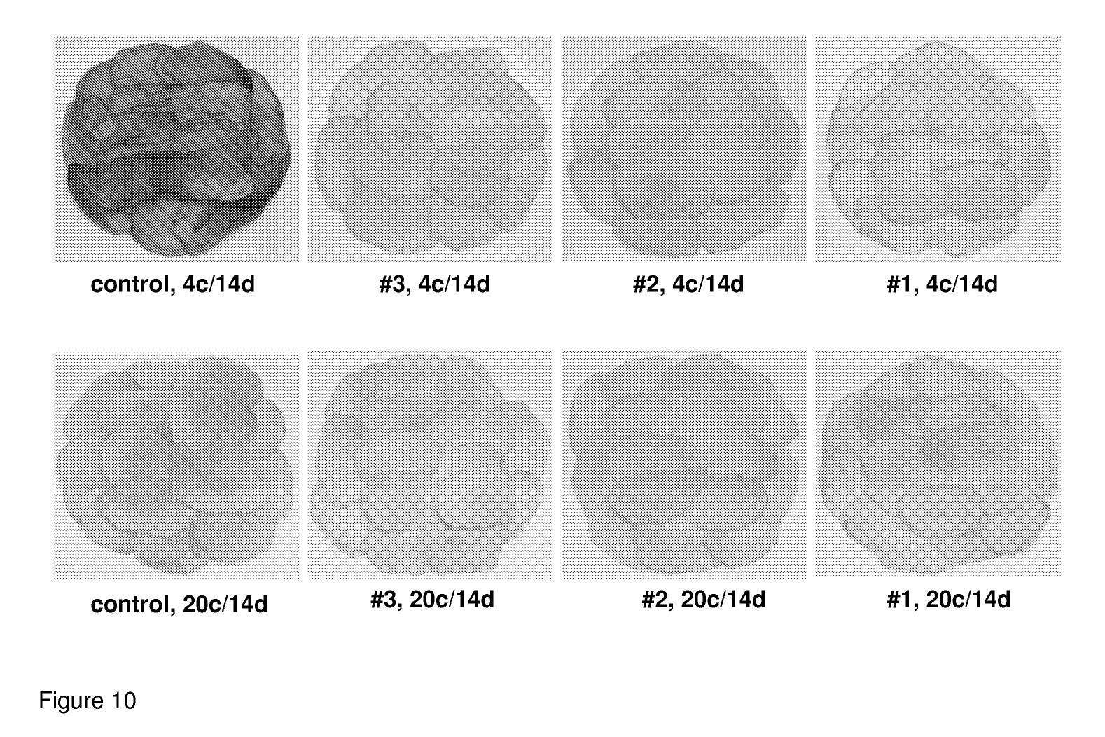

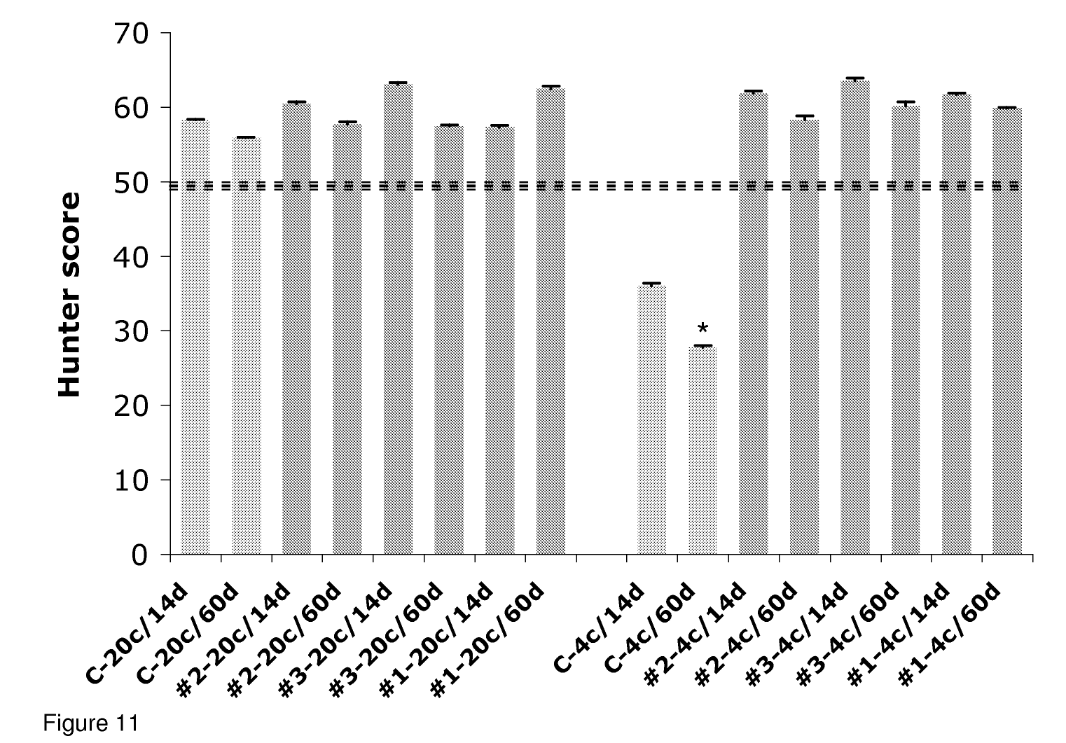

Chipping experiments on field grown VI-RNAi tubers described above at 14-day cold storage along with controls. The chipping experiments were performed as described in Example 4. Good chip scores (4.5) were obtained for 14-day cold stored VI-RNAi tuber samples (See, FIG. 10), where as cold stored control tubers produced poor chip scores (≧8.0). The color of potato chips was validated using a Hunterlab Colorflex calorimetric spectrophotometer. This equipment measures true color of potato chips (an average of 40 chips) by using preset three dimensional color scales. A Hunter value of ≧50 or higher is widely accepted chip color score in potato processing industry. Hunter values of >60 for all the 14-day cold stored VI-RNAi tubers were obtained compared to value of 36.01±0.35 for control tubers (See, FIG. 11). Similarly, Hunter values of >58 were obtained for all the 60-day cold stored VI-RNAi tubers compared to the value of 27.77±0.28 for control tubers (See, FIG. 11).

### Acrylamide Analysis for Field Grown Tubers

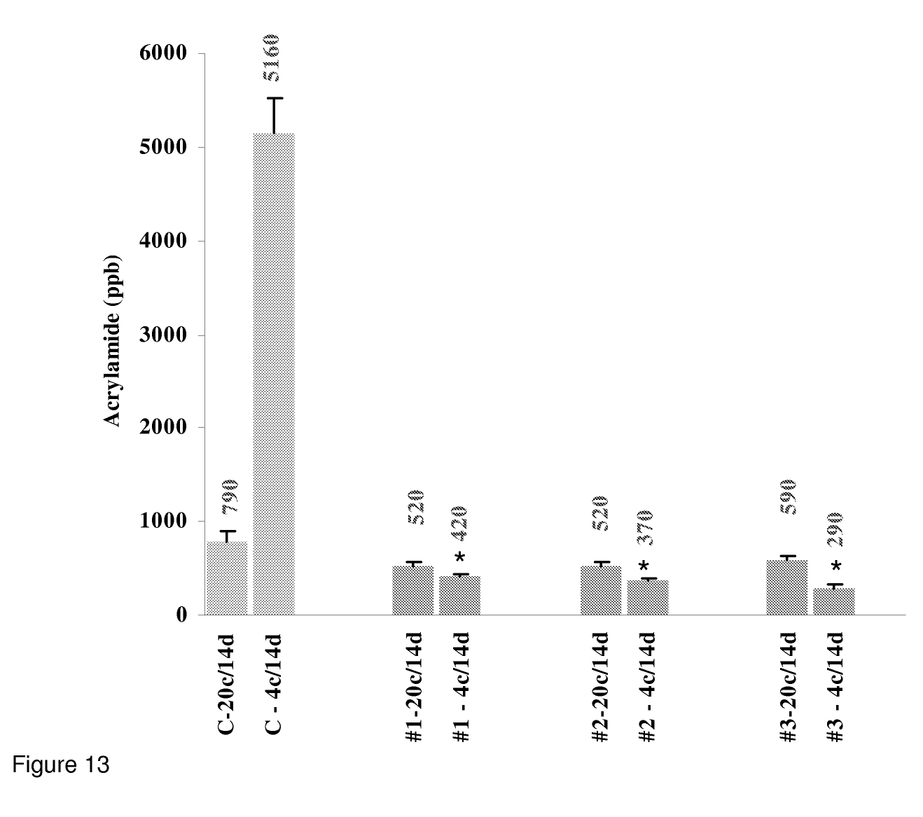

Acrylamide analyses were performed on potato chips processed from room temperature (RT) and 14-day cold stored field harvested tubers described above using the techniques as described in Example 5. Substantial acrylamide reductions of around 100-fold were observed among cold-stored RNAi tubers compared to cold-stored control tubers. Significantly (p<0.05) acrylamide values for tubers from line #2 and #1 were 180 and 650 ppb compared to 29550 ppb among the controls (See, FIG. 13).

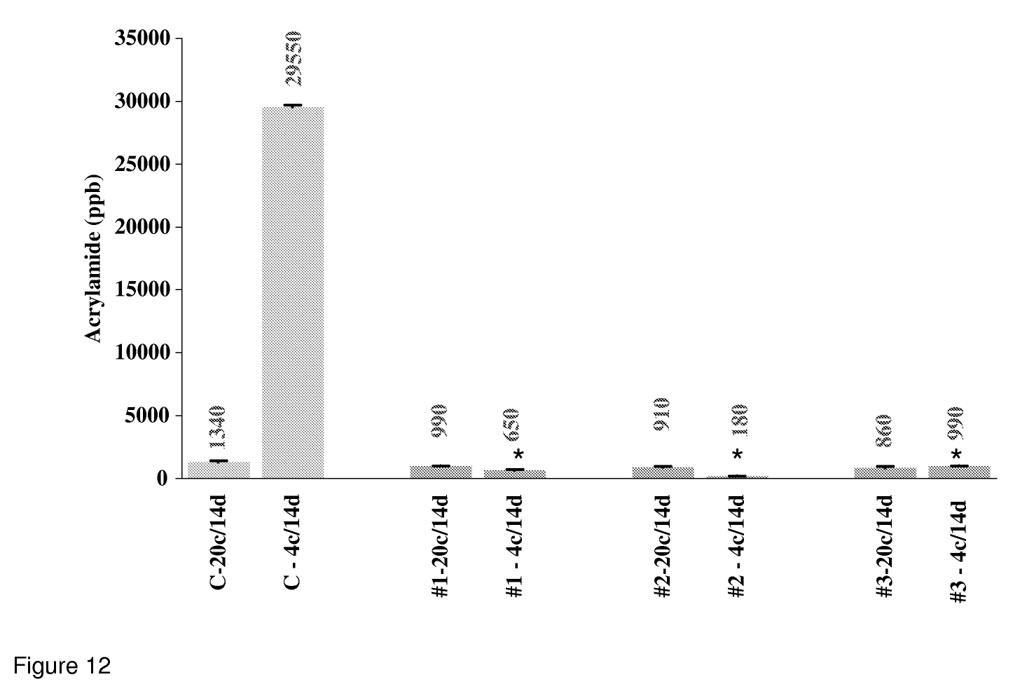

Acrylamide levels among both field and greenhouse grown (See, FIGS. 12 and 13) transgenic tubers after 14-day cold storage consistently produced very low acrylamide levels.

One skilled in the art would readily appreciate that the present disclosure is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

### REFERENCES

The disclosures of all references are herein incorporated by reference in their entireties.
Ausubel, F. M. 1987. Current protocols in molecular biology. Greene Publishing Associates; J. Wiley, order fulfillment, Brooklyn, N.Y. Media, Pa. 2 v. (loose-leaf) pp.
Beclin, C., S. Boutet, P. Waterhouse, et al. 2002. A branched pathway for transgene-induced RNA silencing in plants. Curr Biol. 12:684-8.
Benson, D. A., M. Boguski, D. J. Lipman, et al. 1994. GenBank. Nucleic Acids Res. 22:3441-4.
Bhaskar, P. B., J. A. Raasch, L. C. Kramer, et al. 2008. Sgt1, but not Rar1, is essential for the RB-mediated broad-spectrum resistance to potato late blight. BMC Plant Biol. 8:8.
Brummell, D., P. Balint-Kurti, M. Harpster, et al. 2003. Inverted repeat of a heterologous 3′-untranslated region for high-efficiency, high-throughput gene silencing. Plant J. 33:798-800.
Capecchi, M. R. 1980. High efficiency transformation by direct microinjection of DNA into cultured mammalian cells. Cell. 22:479-88.
Chen, X., F. Salamini, and C. Gebhardt. 2001. A potato molecular-function map for carbohydrate metabolism and transport. TAG Theoretical and Applied Genetics. 102:284-295.
Chuang, C. F., and E. M. Meyerowitz. 2000. Specific and heritable genetic interference by double-stranded RNA in Arabidopsis thaliana. Proc Natl Acad Sci USA. 97:4985-90.
Clapp, D. W. 1993. Somatic gene therapy into hematopoietic cells. Current status and future implications. Clin Perinatol. 20:155-68.
Council for Agricultural Science and Technology (CAST). 2006. Acrylamide in Food. Issue Paper 32. CAST, Ames, Iowa
Curiel, D. T., S. Agarwal, E. Wagner, et al. 1991. Adenovirus enhancement of transferrin-polylysine-mediated gene delivery. Proc Natl Acad Sci USA. 88:8850-4.
Curiel, D. T., E. Wagner, M. Cotten, et al. 1992. High-efficiency gene transfer mediated by adenovirus coupled to DNA-polylysine complexes. Hum Gene Ther. 3:147-54.
Dafny-Yelin, M., and T. Tzfira. 2007. Delivery of multiple transgenes to plant cells. Plant Physiol. 145:1118-28.
Dale, M. F., and J. E. Bradshaw. 2003. Progress in improving processing attributes in potato. Trends Plant Sci. 8:310-2.
Di Serio, F., H. Schob, A. Iglesias, et al. 2001. Sense- and antisense-mediated gene silencing in tobacco is inhibited by the same viral suppressors and is associated with accumulation of small RNAs. Proc Natl Acad Sci USA. 98:6506-10.
Douches, D., and R. Freyer. 1994. Identification of genetic factors influencing chip color in diploid potato. Amer J Potato Res. 71:581-591.
Eglitis, M. A., and W. F. Anderson. 1988. Retroviral vectors for introduction of genes into mammalian cells. Biotechniques. 6:608-14.
Eglitis, M. A., P. W. Kantoff, D. B. Kohn, et al. 1988. Retroviral-mediated gene transfer into hemopoietic cells. Adv Exp Med. Biol. 241:19-27.
Food Navigator (2005) http://www.foodnavigator.com/Financial-Industry/California-sues-food-firms-over-acrylamide
Fromm, M., L. P. Taylor, and V. Walbot. 1985. Expression of genes transferred into monocot and dicot plant cells by electroporation. Proc Natl Acad Sci USA. 82:5824-8.
Fynan, E. F., R. G. Webster, D. H. Fuller, et al. 1993. DNA vaccines: protective immunizations by parenteral, mucosal, and gene-gun inoculations. Proc Natl Acad Sci USA. 90:11478-82.
Gendler, K., T. Paulsen, and C. Napoli. 2008. Chrom DB: the chromatin database. Nucleic Acids Res. 36:D298-302.
Graham, F. L., and A. J. van der Eb. 1973. Transformation of rat cells by DNA of human adenovirus 5. Virology. 54:536-9.
Granath F and Tomqvist M (2003) who knows whether acrylamide in food is hazardous to humans? Nat'l. Cancer Inst. 95: 842-843.
Greiner, S., T. Rausch, U. Sonnewald, et al. 1999. Ectopic expression of a tobacco invertase inhibitor homolog prevents cold-induced sweetening of potato tubers. Nat. Biotechnol. 17:708-11.
Grob K, Biedermann M, Biedermann-Brem S, Noti A, Imhof D, Amrein T, Pfefferle Bazzocco D (2003) French fries with less than 100 mg/kg acrylamide. Collaboration between cooks and analysts. Eur Food Res Technol 217:185-194.
Guo, H.-S., J.-F. Fei, Q. Xie, et al. 2003. A chemical-regulated inducible RNAi system in plants. Plant J. 34:383-392.
Hannon, G. J. 2002. RNA interference. Nature. 418:244-51.
Hellens, R., P. Mullineaux, and H. Klee. 2000. A guide to Agrobacterium binary Ti vectors. Trends Plant Sci. 5:446-51.
Helliwell, C. A., S. V. Wesley, and P. Waterhouse. U.S. Pat. No. 6,933,146. 2005. Methods and means for producing efficient silencing construct using recombinational cloning.
Herdewijn, P. 2000. Heterocyclic modifications of oligonucleotides and antisense technology. Antisense Nucleic Acid Drug Dev. 10:297-310.
Johnston, S. A., and D. C. Tang. 1994. Gene gun transfection of animal cells and genetic immunization. Methods Cell Biol. 43 Pt A:353-65.
Jorgensen, R., C. Snyder, and J. Jones. 1987. T-DNA is organized predominatly in inverted repeat structures in plants transformed with Agrobacterium tumefaciens C58 derivatives. Mol. Gen. Genetics. 207:471-477.
Keijbets, M. 2008. Potato processing for the consumer: Developments and future challenges. Potato Rsrch. 51:271-281.
Kusaba, M. 2004. RNA interference in crop plants. Curr Opin Biotechnol. 15:139-43.
Llave, C., K. D. Kasschau, M. A. Rector, et al. 2002. Endogenous and silencing-associated small RNAs in plants. Plant Cell. 14:1605-19.
Lu, L., M. Xiao, D. W. Clapp, et al. 1993. High efficiency retroviral mediated gene transduction into single isolated immature and replatable CD34(3+) hematopoietic stem/progenitor cells from human umbilical cord blood. J Exp Med. 178:2089-96.
Menendez, C. M., E. Ritter, R. Schafer-Pregl, et al. 2002. Cold sweetening in diploid potato: mapping quantitative trait loci and candidate genes. Genetics. 162:1423-34.
Mottram D S, Wedzicha B L, Dodson A T (2002) Acrylamide is formed in the Maillard reaction. Nature 419: 448-449.
Omirulleh, S., M. Abraham, M. Golovkin, et al. 1993. Activity of a chimeric promoter with the doubled CaMV 35S enhancer element in protoplast-derived cells and transgenic plants in maize. Plant Mol Biol. 21:415-28.
Park, W., J. Li, R. Song, et al. 2002. CARPEL FACTORY, a Dicer homolog, and HEN1, a novel protein, act in microRNA metabolism in Arabidopsis thaliana. Curr Biol. 12:1484-95.
Park Y W, Yang H W, Storkson J M, Albright K J, Liu W, Lindsay R C, Pariza M W (2005) Controlling acrylamide in French fry and potato chip models and a mathematical model of acrylamide formation. In: Friedman M and Mottram D (eds), Chemistry and safety of acrylamide in food, Springer, New York, pp. 343-356.
Pelucchi C, Franceschi S, Levi F, Trichopoulos D, Bosetti C, Negri E, Vecchia C L (2003) Fried potatoes and human cancer. Int. J. Cancer 105: 558-560.
Preuss, S., and C. Pikaard. 2004. TARGETED GENE SILENCING IN PLANTS USING RNA INTERFERENCE. In RNA Interference (RNAi)˜Nuts & Bolts of siRNA Technology Vol. D. Engelke, editor DNA Press, Glendale, Calif. 23-36.
Quackenbush, J., F. Liang, I. Holt, et al. 2000. The TIGR gene indices: reconstruction and representation of expressed gene sequences. Nucleic Acids Res. 28:141-5.
Rausch, T., and S. Greiner. 2004. Plant protein inhibitors of invertases. Biochim Biophys Acta. 1696:253-61.
Reeves, A. 1982. Potato chip color ratings of advanced selections from the Maine potato breeding program. Amer J Potato Res. 59:389-394.

Rhoades, M. W., B. J. Reinhart, L. P. Lim, et al. 2002. Prediction of plant microRNA targets. Cell. 110:513-20.
Rogers, S. G., H. J. Klee, R. B. Horsch, et al. 1987. Improved vectors for plant transformation: expression cassette vectors and new selectable markers. Methods Enzymol. 153:253-277.
Ruth, J. U.S. Pat. No. 4,948,882. 1990. Ruth, J. 1990. SINGLE-STRANDED LABELELED OLIGONUCLEOTIDES, REACTIVE MONOMERS AND METHODS OF SYNTHESIS.
Sambrook, J., and D. W. Russell. 2001. Molecular cloning: a laboratory manual. Cold Spring Harbor Laboratory Press, Cold Spring Harbor, N.Y.
San Francisco Chronicle (2008) http://www.sfgate.com/cgibin/article.cgi?f4c/a/2008/08/02/BAFE1244HA.DTL.
Schuster R (1988) Determination of amino acids in biological, pharmaceutical, plant and food samples by automated precolumn derivatization and high-performance liquid chromatography. J Chromatography 431: 271-284.
Smith, N. A., S. P. Singh, M. B. Wang, et al. 2000. Total silencing by intron-spliced hairpin RNAs. Nature. 407:319-20.
Song, J., J. M. Bradeen, S. K. Naess, et al. 2003. Gene R B cloned from Solanum bulbocastanum confers broad spectrum resistance to potato late blight. Proc Natl Acad Sci USA. 100:9128-33.
Sowokinos, J. 2001. Biochemical and molecular control of cold-induced sweetening in potatoes. Amer J Potato Res. 78:221-236.
Sowokinos, J. 2007. The canon of potato science: 38. Carbohydrate metabolism. Potato Rsrch. 50:367-370.
Spielmann, A., and R. Simpson. 1986. T-DNA structure in transgenic tobacco plants with multiple independent integration sites Mol. Gen. Genetics. 205:34-41.
Tareke E, Rydberg P, Karlsson S, Tornquist M (2002) Analysis of acrylamide, a carcinogen formed in heated foodstuffs. J Agric Food Chem 50: 4998-5006.
Wagner, E., K. Zatloukal, M. Cotten, et al. 1992. Coupling of adenovirus to transferrin-polylysine/DNA complexes greatly enhances receptor-mediated gene delivery and expression of transfected genes. Proc Natl Acad Sci USA. 89:6099-103.
Waterhouse, P. M., and C. A. Helliwell. 2003. Exploring plant genomes by RNA-induced gene silencing. Nat Rev Genet. 4:29-38.
Wesley, S. V., C. A. Helliwell, N. A. Smith, et al. 2001. Construct design for efficient, effective and high-throughput gene silencing in plants. Plant J. 27:581-90.
Wong, T. K., and E. Neumann. 1982. Electric field mediated gene transfer. Biochem Biophys Res Commun. 107:584-7.
Zamore, P. D., T. Tuschl, P. A. Sharp, et al. 2000. RNAi: double-stranded RNA directs the ATP-dependent cleavage of mRNA at 21 to 23 nucleotide intervals. Cell. 101:25-33.
Zatloukal, K., E. Wagner, M. Cotten, et al. 1992. Transferrinfection: a highly efficient way to express gene constructs in eukaryotic cells. Ann N Y Acad. Sci. 660:136-53.
Zeigelhoffer, T., J. Will, and S. Austin-Phillips. 1999. Expression of bacterial cellulase genes in transgenic alfalfa (Medicago sativa L.), potato (Solanum tuberosum L.), and tobacco (Nicotiana tabacum L.). Mol. Breed. 5:309-318.
Zrenner, R., K. Schuler, and U. Sonnewald. 1996. Soluble acid invertase determines the hexose-to-sucrose ratio in cold-stored potato tubers. Planta. 198:246-52.

What is claimed is:

1. An isolated polynucleotide comprising a nucleic acid sequence having at least 90% nucleic acid sequence identity with a sequence selected from the group consisting of SEQ ID NOs: 5, 6, 9, 10, 11, 23 and 24.

2. The polynucleotide of claim 1, wherein the sequence has at least 95% nucleic acid sequence identity.

3. The isolated polynucleotide of claim 1, comprising a nucleic acid sequence selected from the group consisting of SEQ ID NOs: 5, 6, 9, 10, 11, 23 and 24.

4. An RNAi vector comprising a polynucleotide of claim 1.

5. A transgenic plant comprising the vector of claim 4.

6. The transgenic plant of claim 5, wherein the plant is Solanum sp.

7. The transgenic plant of claim 6, wherein the plant is Solanum tuberosum.

8. The transgenic plant of claim 7, wherein the expression of a vacuolar invertase gene is decreased by at least 90% when compared to a non-transformed plant.

9. The transgenic plant of claim 7, wherein the plant comprises a tuber.

10. The transgenic plant of claim 9, wherein the tuber has been processed into a crisp, chip, French fry, potato stick, shoestring potato or other potato product.

11. A method for silencing vacuolar invertase in a transgenic plant, wherein the plant is a potato plant, a sweet potato plant, a yam or a Cassaya, comprising decreasing the level of VI activity compared to its level in a control, non-transgenic potato, sweet potato, yam or Cassaya plant by reducing the level of an mRNA in the transgenic potato plant, wherein the mRNA is encoded by a polynucleotide having at least 90% sequence identity to a nucleic acid sequence of SEQ ID NO:4, and by expression of an RNAi construct comprising a fragment of at least 20 contiguous nucleotides of a sequence having at least 90% sequence identity to SEQ ID NO:4.

12. The method of claim 11, wherein the RNAi construct comprises a polynucleotide having at least 90% sequence identity to a polynucleotide selected from the group consisting of SEQ ID NOs: 5, 6, 9, 10, 11, 23 and 24.

13. The method of claim 11, further comprising the step of screening the transgenic plants for a reduction in VI activity by comparing the VI activity in the transgenic plant to a control plant.

14. The method of claim 11, further comprising the step of screening potatoes or sweet potatoes produced by the transgenic plants by comparing a transgenic potato or sweet potato with a control potato or sweet potato for cold storage-induced sweetening.

15. The method of claim 14, wherein the screening comprises assaying chip color after frying.

16. A method for controlling the accumulation of reducing sugars in a potato or sweet potato plant during cold storage, the method comprising: decreasing a level of vacuolar invertase activity in the potato or sweet potato plant relative to a control potato or sweet potato plant by introducing to the potato plant an RNAi construct comprising a fragment of at least 20 contiguous nucleotides of a sequence having at least 90% sequence identity to SEQ ID NO:4, and maintaining the plant under conditions sufficient for expression of the RNAi construct thereby decreasing the level of an mRNA that is encoded by a polynucleotide having at least 90% sequence identity to a nucleic acid sequence of SEQ ID NO:4.

17. The method of claim 16, further comprising assaying the color of a potato product from a potato of the plant after heat processing the potato.

18. The method of claim 16, wherein assaying the color of the potato or sweet potato product comprises comparing the product color with the color of a control potato or sweet potato product from a control potato or sweet potato plant.

19. The method of claim 16, further comprising heat processing the potato into a crisp, chip, French fry, potato stick, shoestring potato or other potato product or sweet potato product.

20. The method of claim 16, wherein the RNAi construct comprises a polynucleotide having at least 90% sequence identity to a polynucleotide selected from the group consisting of SEQ ID NOs: 5, 6, 9, 10, 11, 23 and 24.

21. A method for controlling acrylamide formation during heat processing of a potato or sweet potato from a potato or sweet potato plant, the method comprising: decreasing a level of vacuolar invertase activity in the potato or sweet potato plant relative to a control potato or sweet potato plant by introducing to the potato plant an RNAi construct comprising a fragment of at least 20 contiguous nucleotides of a sequence having at least 90% sequence identity to SEQ ID NO:4, and maintaining the plant under conditions sufficient for expression of the RNAi construct thereby decreasing the level of an mRNA that is encoded by a polynucleotide having at least 90% sequence identity to a nucleic acid sequence of SEQ ID NO:4.

22. The method of claim 21, further comprising assaying the level of acrylamide in a heat processed potato product of the potato or sweet potato product of the sweet potato.

23. The method of claim 22, wherein assaying the level of acrylamide in the potato product or sweet potato product comprises comparing the acrylamide level with an acrylamide level in a control potato product from a control potato plant or a control sweet potato product from a control sweet potato product.

24. The method of claim 22, further comprising heat processing the potato into a crisp, chip, French fry, potato stick, shoestring potato or other potato product or sweet potato into a sweet potato product.

25. The method of claim 22, wherein the RNAi construct comprises a polynucleotide having at least 90% sequence identity to a polynucleotide selected from the group consisting of SEQ ID NOs: 5, 6, 9, 10, 11, 23 and 24.


SEQUENCE LISTING (26 sequences)

SEQ ID NO: 1 - DNA, 2351 bp, Solanum tuberosum
gcacgagtatggccacccagtaccattccagttatgacccggaaaactccgcctcccattacacattcctcccggatcaacccgattccggccaccggaagtcccttaaaatcatctccggcattttcctctcctctttccttttgctttctgtagccttctttccgatcctcaacaaccagtcaccggacttgcagagtaactcccgttcgccggcgccgccgtcaagaggtgtttctcagggagtctccgataagacttttcgagatgtcgtcaatgctagtcacgtttcttatgcgtggtccaatgctatgcttagctggcaaagaactgcttaccattttcaacctcaaaaaaattggatgaacgatcctaatggtccattgtaccacaagggatggtatcatcttttttatcaatacaatccagattcagctatttggggaaatatcacatggggccatgccgtatccaaggacttgatccactggctctacttgccttttgccatggttcctgatcaatggtacgatataaacggtgtctggactgggtccgctaccatcctacccgatggtcagatcatgatgctttataccggtgacactgatgattatgtacaagtgcaaaatcttgcgtaccccaccaacttatctgatcctctccttctagactgggtcaagtacaaaggcaacccggttctggttcctccacccggcattggtgtcaaggactttagagacccgaccactgcttggaccggaccccaaaatgggcaatggcttttaacaatcgggtctaagattggtaaaacgggtattgcacttgtttatgaaacttccaacttcacaagctttaagctattggatgaagtgctgcatgcggttccgggtacgggtatgtgggagtgtgtggacttttacccggtatcgactgaaaaaacaaacgggttggacacatcatataacggcccgggtgtaaagcatgtgttaaaagcaagtttagatgacaataagcaagatcactatgctattgggacgtatgacttgacaaagaacaaatggacacccgataacccggaattggattgtggaattgggttgaagctggattatgggaaatattatgcatcaaagacattttatgacccgaagaaacaacgaagagtactgtggggatggattggggaaactgatagtgaatctgctgacctgcagaagggatgggcatctgtacagagtattccaaggacagtgctttacgacaagaagacagggacacatctacttcagtggccagttgaagaaattgaaagcttaagagcgggtgatcctattgttaagcaagtcaatcttcaaccaggttcaattgagctactccatgttgactcagctgcagagttggatatagaagcctcatttgaagtggacaaagtcgcgctccagggaataattgaagcagatcatgtaggtttcagctgctctactagtggaggtgctgctagcagaggcattttgggaccatttggtgtcgttgtaattgctgatcaaacgctatctgagctaacgccagtttacttctacatttctaaaggagctgatggccgagctgagactcacttctgtgctgatcaaaccagatcctcagaggctccgggagttgctaaacaagtttatggtagttcagtacccgtgttggacggtgaaaaacattcgatgagattattggtggaccactcaattgtggagagctttgctcaaggaggaagaacagtcataacatcgcgaatttacccaacaaaggcagtgaatggagcagcacgactcttcgttttcaacaatgccacaggggctagcgtgactgcctccgtcaagatttggtcacttgagtcggctaatattcgatccttccccttgcaagacttgtaattcatcaagccatatcttcttcattctttttttcatttgaaggttatttcaccgatgtcccatcaagaaagggaagagagggagaatatgtagtgttatactctacttattcgccattttagtgatttttctactggacttttgctattcgccataaggtttagttgttgtctagcaatgtcagcagcggggcggatctatagtgtaatgtatgggttcctggaaaccgaataggtcttacttggattttatgtaaactaagaaaattcagcaaatacatacaaataatttatcgatttcttattgctggtgaggattcggttccctggcagttacaaaactaaccatgggcacctaaatacttggggcaacgagattgacatttgagcttatgcagttgcttagagcacgtgatttcgccgg

SEQ ID NO: 2 - DNA, 2296 bp, Solanum tuberosum
gcacgagtatggccacgcagtaccattccagttatgacccggaaaactccgcctcccattacacattcctcccggatcaacccgattccggccaccggaagtcccttaaaatcatctccggcattttcctctcctctttccttttgctttctgtagccttctttccgatcctcaacaaccagtcaccggacttgcagagtaactcccgttcgccggcgccgccgtcaagaggtgtttctcagggagtctccgataagacttttcgagatgtcgtcaatgctagtcacgtttcttatgcgtggtccaatgctatgcttagctggcaaagaactgcttaccattttcaacctcaaaaaaattggatgaacgatcctaatggtccattgtaccacaagggatggtatcatcttttttatcaatacaatccagattcagctatttggggaaatatcacatggggccatgccgtatccaaggacttgatccactggctctacttgccttttgccatggttcctgatcaatggtacgatattaacggtgtctggactgggtccgctaccatcctacccgatggtcagatcatgatgctttataccggtgacactgatgattatgtacaagtgcaaaatcttgcgtaccccactaacttatctgatcctctccttctagactgggtcaagtacaaaggcaacccggttctggttcctccacccggcattggtgtcaaggactttagagacccgaccactgcttggaccggaccccaaaatgggcaatggcttttaacaatcgggtctaagattggtaaaacgggtattgcacttgtttatgaaacttccaacttcacaagctttaagctattggatgaagtgctgcatgcggttccgggtacgggtatgtgggagtgtgtggacttttacccggtatcgactgaaaaaacaaacgggttggacacatcatataacggcccgggtgtaaagcatgtgttaaaagcaagtttagatgacaataagcaagatcactatgctattgggacgtatgacttgacaaagaacaaatgcacacccgataacccggaattggattgtggaattgggttgaagctggattatgggaaatattatgcatcaaagacattttatgacccgaagaaacaacgaagagtactgtggggatggattggggaaactgatagtgaatctgctgacctgcagaagggatgggcatctgtacagagtattccaaggacagtgctttacgacaagaagacagggacacatctacttcagtggccagttgaagaaattgaaagcttaagaggcggtgatcctattgttaagcaagtcaatcttcaaccaggttcaattgagctactccatgttgactcagctgcagagttggatatagaagcctcatttgaagtggacaaagtcgcgctccagggaataattgaagcagatcatgtaggtttcagctgctctactagtggaggtgctgctagcagaggcattttgggaccatttggtgtcgttgtaattgctgatcaaaagctatctgacgtaacgccagtttacttctacatttctaaaggagctgatggtcgagctgagactcacttctgtgctgatcaaactagatcctcagaggctccgggagttgctaaacaagtttatggtagttcagtacccgtgttggacggtgaaaaacattcgatgagattattggtggaccactcaattgtggagagctttgctcaaggaggaagaacagtcataacatcgcgaatttacccaacaaaggcagtgaatggagcagcacgactcttcgttttcaacaatcgcacaggggctagcgtgactgcctccgtcaagatttggtcacttgagtcggctaatattcgatccttccccttgcaagacttgtaattcatcaagccatatcttcttcattctttttttcatttgaaggttatttcaccgatgtcccatcaagaaagggaagagagggagaatatgtagtgttatactctacttattcgccattttagtgatttttctactggacttttgctattcgccataaggtttagttgttgtctagcaatgtcagcagcggggcggatctatagtgtaatgtatgggttcctggaaaccgaataggtcttacttggattttatgtaaactaagaaaattcagcaaatacatacaaataatttatcgatttcttattgctggtgaggattcggttccctggcagttagaaaactgacatgggcacctaaatatttggggc

SEQ ID NO: 3 - DNA, 1920 bp, Solanum tuberosum
atggccacgcagtaccattccagttatgacccggaaaactccgcctcccattacacattcctcccggatcaacccgattccggccaccggaagtcccttaaaatcatctccggcattttcctctcctctttccttttgctttctgtagccttctttccgatcctcaacaaccagtcaccggacttgcagagtaactcccgttcgccggcgccgccgtcaagaggtgtttctcagggagtctccgataagacttttcgagatgtcgtcaatgctagtcacatttcttatgcgtggtccaatgctatgcttagctggcaaagaactgcttaccattttcaacctcaaaaaaattggatgaacgatcctaatggtccattgtaccacaagggatggtatcatcttttttatcaatacaatccagattcagctatttggggaaatatcacatggggccatgccgtatccaaggacttgatccactggctctacttgccttttgccatggttcctgatcaatggtacgatataaacggtgtctggactgggtccgctaccatcctacccgatggtcagatcatgatgctttataccggtgacactgatgattatgtacaagtgcaaaatcttgcgtaccccaccaacttatctgatcctctccttctagactgggtcaagtacaaaggcaacccggttctggttcctccacccggcattggtgtcaaggactttagagacccgaccactgcttggaccggaccccaaaatgggcaatggcttttaacaatcgggtctaagattggtaaaacgggtattgcacttgtttatgaaacttccaacttcacaagctttaagctattgggtgaagtgctgcatgcggttccgggtacgggtatgtgggagtgtgtggacttttacccggtatcgactgaaaaaacaaacgggttggacacatcatataacggcccgggtgtaaagcatgtgttaaaagcaagtttagatgacaataagcaagatcactatgctattgggacgtatgacttgacaaagaacaaatggacacccgataacccggaattggattgtggaattgggttgaagctggattatgggaaatattatgcatcaaagacattttatgacccgaagaaacaacgaagagtactgtggggatggattggggaaactgacagtgaatctgctgacctgcagaagggatgggcatctgtacagagtattccaaggacagtgctttacgacaagaagacagggacacatctacttcagtggccagttgaagaaattgaaagcttaagagcgggtgatcctattgttaagcaagtcaatcttcaaccaggttcaattgagctactccatgttgactcagctgcagagttggatatagaagcctcatttgaagtggacaaagtcgcgctccagggaataattgaagcagatcatgtaggtttcagctgctctactagtggaggtgctgctagcagaggcattttgggaccatttggtgtcgttgtaattgctgatcaaacgctatctgagctaacgccagtttacttcttcatttctaaaggagctgatggccgagctgagactcacttctgtgctgatcaaaccagatcctcagaggctccgggagttgctaaacaagtttatggtagttcagtacccgtgttggacggtgaaaaacattcgatgagattattggtggaccactcaattgtggagagctttgctcaaggaggaagaacagtcataacatcgcgaatttacccaacaaaggcagtgaatggagcagcacgactcttcgttttcaacaatgccacaggggctagcgtgactgcctccgtcaagatttggtcacttgagtcggctaatattcgatccttccccttgcaagacttgtaa

SEQ ID NO: 4 - DNA, 2351 bp, Solanum tuberosum
gcacgagtatggccacccagtaccattccagttatgacccggaaaactccgcctcccattacacattcctcccggatcaacccgattccggccaccggaagtcccttaaaatcatctccggcattttcctctcctctttccttttgctttctgtagccttctttccgatcctcaacaaccagtcaccggacttgcagagtaactcccgttcgccggcgccgccgtcaagaggtgtttctcagggagtctccgataagacttttcgagatgtcgtcaatgctagtcacgtttcttatgcgtggtccaatgctatgcttagctggcaaagaactgcttaccattttcaacctcaaaaaaattggatgaacgatcctaatggtccattgtaccacaagggatggtatcatcttttttatcaatacaatccagattcagctatttggggaaatatcacatggggccatgccgtatccaaggacttgatccactggctctacttgccttttgccatggttcctgatcaatggtacgatataaacggtgtctggactgggtccgctaccatcctacccgatggtcagatcatgatgctttataccggtgacactgatgattatgtacaagtgcaaaatcttgcgtaccccaccaacttatctgatcctctccttctagactgggtcaagtacaaaggcaacccggttctggttcctccacccggcattggtgtcaaggactttagagacccgaccactgcttggaccggaccccaaaatgggcaatggcttttaacaatcgggtctaagattggtaaaacgggtattgcacttgtttatgaaacttccaacttcacaagctttaagctattggatgaagtgctgcatgcggttccgggtacgggtatgtgggagtgtgtggacttttacccggtatcgactgaaaaaacaaacgggttggacacatcatataacggcccgggtgtaaagcatgtgttaaaagcaagtttagatgacaataagcaagatcactatgctattgggacgtatgacttgacaaagaacaaatggacacccgataacccggaattggattgtggaattgggttgaagctggattatgggaaatattatgcatcaaagacattttatgacccgaagaaacaacgaagagtactgtggggatggattggggaaactgatagtgaatctgctgacctgcagaagggatgggcatctgtacagagtattccaaggacagtgctttacgacaagaagacagggacacatctacttcagtggccagttgaagaaattgaaagcttaagaggcggtgatcctattgttaagcaagtcaatcttcaaccaggttcaattgagctactccatgttgactcagctgcagagttggatatagaagcctcatttgaagtggacaaagtcgcgctccagggaataattgaagcagatcatgtaggtttcagctgctctactagtggaggtgctgctagcagaggcattttgggaccatttggtgtcgttgtaattgctgatcaaacgctatctgagctaacgccagtttacttctacatttctaaaggagctgatggccgagctgagactcacttctgtgctgatcaaaccagatcctcagaggctccgggagttgctaaacaagtttatggtagttcagtacccgtgttggacggtgaaaaacattcgatgagattattggtggaccactcaattgtggagagctttgctcaaggaggaagaacagtcataacatcgcgaatttacccaacaaaggcagtgaatggagcagcacgactcttcgttttcaacaatgccacaggggctagcgtgactgcctccgtcaagatttggtcacttgagtcggctaatattcgatccttccccttgcaagacttgtaattcatcaagccatatcttcttcattctttttttcatttgaaggttatttcaccgatgtcccatcaagaaagggaagagagggagaatatgtagtgttatactctacttattcgccattttagtgatttttctactggacttttgctattcgccataaggtttagttgttgtctagcaatgtcagcagcggggcggatctatagtgtaatgtatgggttcctggaaaccgaataggtcttacttggattttatgtaaactaagaaaattcagcaaatacatacaaataatttatcgatttcttattgctggtgaggattcggttccctggcagttacaaaactaaccatgggcacctaaatacttggggcaacgagattgacatttgagcttatgcagttgcttagagcacgtgatttcgccgg

SEQ ID NO: 5 - DNA, 20 bp, Artificial Sequence
  Description of Artificial Sequence Synthetic primer
ttatgcgtggtccaatgcta

SEQ ID NO: 6 - DNA, 20 bp, Artificial Sequence
  Description of Artificial Sequence Synthetic primer
aacccaattccacaatccaa

SEQ ID NO: 7 - DNA, 20 bp, Artificial Sequence
  Description of Artificial Sequence Synthetic primer
caaatggacacccgataacc

SEQ ID NO: 8 - DNA, 20 bp, Artificial Sequence
  Description of Artificial Sequence Synthetic primer
agtcttgcaaggggaaggat

SEQ ID NO: 9 - DNA, 506 bp, Solanum tuberosum
acaggggctagcgtgactgcctccgtcaagatttggtcacttgagtcggctaatattcgatccttccccttgcaagacttgtaattcatcaagccatatcttcttcattctttttttcatttgaaggttatttcaccgatgtcccatcaagaaagggaagagagggagaatatgtagtgttatactctacttattcgccattttagtgatttttctactggacttttgctattcgccataaggtttagttgttgtctagcaatgtcagcagcggggcggatctatagtgtaatgtatgggttcctggaaaccgaataggtcttacttggattttatgtaaactaagaaaattcagcaaatacatacaaataatttatcgatttcttattgctggtgaggattcggttccctggcagttacaaaactaaccatgggcacctaaatacttggggcaacgagattgacatttgagcttatgcagttgcttagagcacgtgatttcgccg

SEQ ID NO: 10 - DNA, 495 bp, Solanum tuberosum
actgggtcaagtacaaaggcaacccggttctggttcctccacccggcattggtgtcaaggactttagagacccgaccactgcttggaccggaccccaaaatgggcaatggcttttaacaatcgggtctaagattggtaaaacgggtattgcacttgtttatgaaacttccaacttcacaagctttaagctattggatgaagtgctgcatgcggttccgggtacgggtatgtgggagtgtgtggacttttacccggtatcgactgaaaaaacaaacgggttggacacatcatataacggcccgggtgtaaagcatgtgttaaaagcaagtttagatgacaataagcaagatcactatgctattgggacgtatgacttgacaaagaacaaatggacacccgataacccggaattggattgtggaattgggttgaagctggattatgggaaatattatgcatcaaagacattttatgacccgaagaaacaacgaagag

SEQ ID NO: 11 - DNA, 508 bp, Solanum tuberosum
gaaagcttaagaggcggtgatcctattgttaagcaagtcaatcttcaaccaggttcaattgagctactccatgttgactcagctgcagagttggatatagaagcctcatttgaagtggacaaagtcgcgctccagggaataattgaagcagatcatgtaggtttcagctgctctactagtggaggtgctgctagcagaggcattttgggaccatttggtgtcgttgtaattgctgatcaaacgctatctgagctaacgccagtttacttctacatttctaaaggagctgatggccgagctgagactcacttctgtgctgatcaaaccagatcctcagaggctccgggagttgctaaacaagtttatggtagttcagtacccgtgttggacggtgaaaaacattcgatgagattattggtggaccactcaattgtggagagctttgctcaaggaggaagaacagtcataacatcgcgaatttacccaacaaaggcagtgaatggagcag

SEQ ID NO: 12 - DNA, 24 bp, Artificial Sequence
  Description of Artificial Sequence Synthetic primer
caccacaggggctagcgtgactgc

SEQ ID NO: 13 - DNA, 22 bp, Artificial Sequence
  Description of Artificial Sequence Synthetic primer
cggcgaaatcacgtgctctaag

SEQ ID NO: 14 - DNA, 24 bp, Artificial Sequence
  Description of Artificial Sequence Synthetic primer
caccactgggtcaagtacaaaggc

SEQ ID NO: 15 - DNA, 23 bp, Artificial Sequence
  Description of Artificial Sequence Synthetic primer
ctcttcgttgtttcttcgggtca

SEQ ID NO: 16 - DNA, 27 bp, Artificial Sequence
  Description of Artificial Sequence Synthetic primer
caccgaaagcttaagaggcggtgatcc

SEQ ID NO: 17 - DNA, 23 bp, Artificial Sequence
  Description of Artificial Sequence Synthetic primer
ctgctccattcactgcctttgtt

SEQ ID NO: 18 - DNA, 20 bp, Artificial Sequence
  Description of Artificial Sequence Synthetic primer
ccaacgctatgtcctgatag

SEQ ID NO: 19 - DNA, 20 bp, Artificial Sequence
  Description of Artificial Sequence Synthetic primer
tttgtcaagaccgacctgtc

SEQ ID NO: 20 - DNA, 506 bp, Artificial Sequence; identical to SEQ ID NO: 9
  Description of Artificial Sequence Synthetic probe

SEQ ID NO: 21 - DNA, 20 bp, Artificial Sequence
  Description of Artificial Sequence Synthetic primer
acaggggctagcgtgactgc

SEQ ID NO: 22 - DNA, 22 bp, Artificial Sequence
  Description of Artificial Sequence Synthetic primer
cggcgaaatcacgtgctctaag

SEQ ID NO: 23 - DNA, 672 bp, Solanum tuberosum
gcacgagtatggccacccagtaccattccagttatgacccggaaaactccgcctcccattacacattcctcccggatcaacccgattccggccaccggaagtcccttaaaatcatctccggcattttcctctcctctttccttttgctttctgtagccttctttccgatcctcaacaaccagtcaccggacttgcagagtaactcccgttcgccggcgccgccgtcaagaggtgtttctcagggagtctccgataagacttttcgagatgtcgtcaatgctagtcacgtttcttatgcgtggtccaatgctatgcttagctggcaaagaactgcttaccattttcaacctcaaaaaaattggatgaacgatcctaatggtccattgtaccacaagggatggtatcatcttttttatcaatacaatccagattcagctatttggggaaatatcacatggggccatgccgtatccaaggacttgatccactggctctacttgccttttgccatggttcctgatcaatggtacgatataaacggtgtctggactgggtccgctaccatcctacccgatggtcagatcatgatgctttataccggtgacactgatgattatgtacaagtgcaaaatcttgcgtaccccaccaacttatctgatcctctccttctag

SEQ ID NO: 24 - DNA, 140 bp, Solanum tuberosum
actgtggggatggattggggaaactgatagtgaatctgctgacctgcagaagggatgggcatctgtacagagtattccaaggacagtgctttacgacaagaagacagggacacatctacttcagtggccagttgaagaaa

SEQ ID NO: 25 - DNA, 20 bp, Artificial Sequence
  Description of Artificial Sequence Synthetic primer
cgctccagggaataattgaa

SEQ ID NO: 26 - DNA, 21 bp, Artificial Sequence
  Description of Artificial Sequence Synthetic primer
tttgtaaactgccagggaacc